(19) United States
(12) Patent Application Publication
(10) Pub. No.: US 2016/0175081 A1
(43) Pub. Date: Jun. 23, 2016

(54) MEDICAL TUBE, MEDICAL DEVICE SET, AND METHOD OF PLACING IMPLANT INDWELLING

(71) Applicant: TERUMO KABUSHIKI KAISHA, Tokyo (JP)

(72) Inventors: Nao MANDAI, Mountain View, CA (US); Masakatsu KAWAURA, Sunnyvale, CA (US)

(73) Assignee: TERUMO KABUSHIKI KAISHA, Tokyo (JP)

(21) Appl. No.: 15/053,467

(22) Filed: Feb. 25, 2016

Related U.S. Application Data
(63) Continuation of application No. PCT/JP2014/073562, filed on Sep. 5, 2014.

(30) Foreign Application Priority Data
Sep. 20, 2013 (JP) 2013-195046

(51) Int. Cl. A61F 2/00; A61M 39/08

(52) U.S. Cl. CPC A61F 2/0036; A61M 39/08; A61F 2250/0078

(57) ABSTRACT

A medical tube is disclosed, which is configured so that an implant to be placed indwelling in a living body can be inserted in the medical tube and includes an extension section which extends the overall length of the medical tube. A medical device set includes the medical tube, and an operation mechanism for operation to actuate an extension mechanism. A method of placing an implant indwelling in a living body, by temporarily inserting the implant in a medical tube, includes preliminarily extending the overall length of the medical tube prior to placing the implant indwelling.

### CROSS-REFERENCES TO RELATED APPLICATIONS

This application is a continuation of International Application No. PCT/JP2014/073562 filed on Sep. 5, 2014, and claims priority to Japanese Application No. 2013-195046 filed on Sep. 20, 2013, the entire content of both of which is incorporated herein by reference.

### TECHNICAL FIELD

The present disclosure generally relates to a medical tube, a medical device set, and a method of placing an implant indwelling.

### BACKGROUND DISCUSSION

If a person suffers from a urinary incontinence, specifically, for example, if a person suffers from a stress urinary incontinence, then urine leakage can be caused by application of abdominal pressure during normal exercise or by laughing, coughing, sneezing or the like. The cause of this may be, for example, that the pelvic floor muscle which is a muscle for supporting the urethra is loosened by birth.

For the treatment of urinary incontinence, a surgical treatment can be effective, in which there is used, for example, a belt-shaped implant called “sling.” The sling is indwelled inside the body and the urethra is supported by the sling (see, for example, Japanese Patent Laid-Open No. 2010-99499). In order to indwell the sling inside the body, an operator would incise the vagina with a surgical knife, dissect the living body tissue between the urethra and vagina, and make the dissected region and the outside communicate with each other through obturator foramens by use of a puncture needle or the like (this operation will hereinafter be referred to as “puncturing operation”). After the puncturing operation, the sling is inserted into the puncture hole formed by the puncturing operation, from the vaginal cavity side, and the sling is placed indwelling in the body.

### SUMMARY

A medical tube, a medical device set, and a method of placing an implant indwelling are disclosed by which an operation of inserting and passing an implant into and through a medical tube can be performed relatively easily and reliably.

A medical tube is disclosed, which is configured so that an implant to be placed indwelling in a living body can be inserted in the medical tube, the medical tube including an extension section which extends an overall length of the medical tube.

Preferably, for example, the medical tube as disclosed above can further include a tube main body including a plurality of tube shaped members interlocked along a longitudinal direction of the medical tube, the tube shaped members communicating with one another. In the medical tube, the extension section can support the adjacent ones of the tube shaped members so that the adjacent ones of the tube shaped members are movable relative to one another along the longitudinal direction.

The medical tube as disclosed above may further include a tube main body including a deformation portion provided at an intermediate part in a longitudinal direction of the medical tube, the deformation portion deformed so as to extend along the longitudinal direction, a deformed state of the deformation portion being maintained. In addition, in the medical tube, the deformation portion functions as the extension section.

In the medical tube as disclosed above, preferably, for example, at least part of the medical tube is rigid, and lumens of the medical tube are communicating after an extending operation by the extension section.

In the medical tube as disclosed above, preferably, for example, the tube main body is configured to be separable, and has a detection section capable of detecting a separation position of the tube main body in a connected state of the tube main body.

In accordance with an exemplary embodiment, a medical device set is disclosed, which can include a medical tube configured so that an implant to be placed indwelling in a living body can be inserted in the medical tube, the medical tube including an extension section which extends an overall length of the medical tube; and an operation mechanism for operation to actuate an extension mechanism.

In the medical device set as disclosed above, preferably, for example, the operation mechanism includes a pressing member which is inserted into the medical tube from a first end side thereof, the pressing member pressing the medical tube toward a second end side thereof.

In the medical device set as disclosed above, preferably, for example, the pressing member is drawn out after an operation of the extension mechanism, and a movement preventing mechanism is provided which prevents the medical tube from moving together with the pressing member when the pressing member is drawn out.

In accordance with an exemplary embodiment, a method is disclosed of placing an implant indwelling in a living body by temporarily inserting the implant in a medical tube, the method including preliminarily extending an overall length of the medical tube prior to placing the implant indwelling.

Depending on the patient's body type, for example, in the case of a comparatively large patient, simply placing a medical tube indwelling in the living body may result in that the medical tube is embedded in the living body due to a restoring force of the living body itself. When the medical tube is embedded in the living body in this way, it is extremely difficult to insert an implant into the medical tube.

In accordance with an exemplary embodiment, according to the described aspects of the present disclosure, the overall length of a medical tube can be extended, so that both end portions of the medical tube in the extended state can be protruded from the living body surface to the outside. Consequently, an operation of inserting the implant into the medical tube can be carried out relatively easily and reliably.

### BRIEF DESCRIPTION OF THE DRAWINGS

FIG. 1 is a perspective view illustrating a case where a medical tube according to a first embodiment of the present disclosure is applied to a puncture apparatus;

FIG. 2 is a lateral view of the puncture apparatus depicted in FIG. 1;

FIG. 3 is a plan view depicting an operating member possessed by the puncture apparatus depicted in FIG. 1;

FIGS. 4A and 4B illustrates a puncture member possessed by the puncture apparatus depicted in FIG. 1, wherein FIG. 4A is a perspective view and FIG. 4B is a sectional view taken along line IVB-IVB of FIG. 4A;

FIG. 5 is a sectional view depicting a guide section of a frame possessed by the puncture apparatus depicted in FIG. 1;

FIG. 6 is a plan view depicting a fixing section of the frame possessed by the puncture apparatus depicted in FIG. 1;

FIG. 7 is a lateral view of an insertion tool possessed by the puncture apparatus depicted in FIG. 1;

FIGS. 8A and 8B illustrate a positional relation of a puncture member and an obturator foramen (pelvis), wherein FIG. 8A is a lateral view and FIG. 8B is a front view;

FIG. 9 is a partial enlarged view of a vaginal-insertion member possessed by the insertion tool depicted in FIG. 7;

FIG. 10A is a sectional view depicting an example of the shape of a vaginal wall;

FIG. 10B is a sectional view depicting a state where the vaginal-insertion member is inserted in a vagina depicted in FIG. 10A;

FIGS. 11A and 11B are views for explaining an operating procedure of the puncture apparatus depicted in FIG. 1;

FIG. 12 is a view for explaining the operating procedure of the puncture apparatus depicted in FIG. 1;

FIG. 13 is a lateral view depicting the relation between the puncture apparatus and the pelvis at the time of the state depicted in FIG. 12;

FIG. 14 is a view (as viewed from the side of the patient's legs) for explaining the operating procedure of the puncture apparatus depicted in FIG. 1;

FIG. 15 is a lateral view depicting the relation between the puncture apparatus and the pelvis at the time of the state depicted in FIG. 14;

FIG. 16 is a view (as viewed from the side of the patient's legs) for explaining the operating procedure of the puncture apparatus depicted in FIG. 1;

FIG. 17 is a view (as viewed from the side of the patient's legs) for explaining the operating procedure of the puncture apparatus depicted in FIG. 1;

FIG. 18 is a view (as viewed from the side of the patient's legs) for explaining the operating procedure of the puncture apparatus depicted in FIG. 1;

FIG. 19 is a view (as viewed from the side of the patient's legs) for explaining the operating procedure of the puncture apparatus depicted in FIG. 1;

FIG. 20 is a view (as viewed from the side of the patient's legs) for explaining the operating procedure of the puncture apparatus depicted in FIG. 1;

FIG. 21 is a view (as viewed from the side of the patient's legs) for explaining the operating procedure of the puncture apparatus depicted in FIG. 1;

FIG. 22 is a sectional view taken along line XXII-XXII of FIG. 14;

FIG. 23 is a sectional view taken along line XXIII-XXIII of FIG. 20;

FIG. 24 is a sectional view taken along line XIV-XIV of FIG. 21;

FIG. 25 is a cross-sectional view of an extension tube possessed by the medical tube depicted in FIG. 1;

FIG. 26 is a cross-sectional view of an extension tube possessed by a medical tube according to a second embodiment of the present disclosure;

FIG. 27 is a cross-sectional view of an extension tube possessed by a medical tube according to a third embodiment of the present disclosure;

FIG. 28 is a cross-sectional view of an extension tube possessed by a medical tube according to a fourth embodiment of the present disclosure;

FIG. 29 is a cross-sectional view of an extension tube possessed by a medical tube according to a fifth embodiment of the present disclosure;

FIG. 30 is a cross-sectional view of an extension tube possessed by a medical tube according to a sixth embodiment of the present disclosure;

FIG. 31 is a cross-sectional view of an extension tube possessed by a medical tube according to a seventh embodiment of the present disclosure;

FIG. 32 is a view (as viewed from the side of the patient's legs) for explaining an operating procedure of a puncture apparatus in the case where a medical tube according to an eighth embodiment of the present disclosure is applied to the puncture apparatus;

FIG. 33 is a view (as viewed from the side of the patient's legs) for explaining the operating procedure of the puncture apparatus in the case where the medical tube according to the eighth embodiment of the present disclosure is applied to the puncture apparatus;

FIG. 34 is a view (as viewed from the side of the patient's legs) for explaining the operating procedure of the puncture apparatus in the case where the medical tube according to the eighth embodiment of the present disclosure is applied to the puncture apparatus;

FIG. 35 is a view (as viewed from the side of the patient's legs) for explaining an operating procedure of a puncture apparatus in the case where a medical tube according to a ninth embodiment of the present disclosure is applied to the puncture apparatus;

FIG. 36 is a view (as viewed from the side of the patient's legs) for explaining the operating procedure of the puncture apparatus in the case where the medical tube according to the ninth embodiment of the present disclosure is applied to the puncture apparatus;

FIG. 37 is a view (as viewed from the side of the patient's legs) for explaining the operating procedure of the puncture apparatus in the case where the medical tube according to the ninth embodiment of the present disclosure is applied to the puncture apparatus;

FIG. 38 is a view (as viewed from the side of the patient's legs) for explaining the operating procedure of the puncture apparatus in the case where the medical tube according to the ninth embodiment of the present disclosure is applied to the puncture apparatus;

FIG. 39 is a view (as viewed from the side of the patient's legs) for explaining the operating procedure of the puncture apparatus in the case where the medical tube according to the ninth embodiment of the present disclosure is applied to the puncture apparatus;

FIG. 40 is a view (as viewed from the side of the patient's legs) for explaining the operating procedure of the puncture apparatus in the case where the medical tube according to the ninth embodiment of the present disclosure is applied to the puncture apparatus;

FIG. 41 is a view (as viewed from the side of the patient's legs) for explaining the operating procedure of the puncture apparatus in the case where the medical tube according to the ninth embodiment of the present disclosure is applied to the puncture apparatus;

FIG. 42 is a view (as viewed from the side of the patient's legs) for explaining the operating procedure of the puncture apparatus in the case where the medical tube according to the ninth embodiment of the present disclosure is applied to the puncture apparatus;

FIG. 43 is a view (as viewed from the side of the patient's legs) for explaining the operating procedure of the puncture apparatus in the case where the medical tube according to the ninth embodiment of the present disclosure is applied to the puncture apparatus;

FIG. 44 is a view (as viewed from the side of the patient's legs) for explaining the operating procedure of the puncture apparatus in the case where the medical tube according to the ninth embodiment of the present disclosure is applied to the puncture apparatus;

FIG. 45 is a view (as viewed from the side of the patient's legs) for explaining the operating procedure of the puncture apparatus in the case where the medical tube according to the ninth embodiment of the present disclosure is applied to the puncture apparatus;

FIGS. 46A and 46B are sectional views taken along line E-E of FIG. 38;

FIGS. 47A to 47C are longitudinal sectional views of a medical tube according to a tenth embodiment of the present disclosure;

FIG. 48 is a view (as viewed from the side of the patient's legs) for explaining an operating procedure of a puncture apparatus in the case where a medical tube according to an eleventh embodiment of the present disclosure is applied to the puncture apparatus;

FIG. 49 is a view (as viewed from the side of the patient's legs) for explaining the operating procedure of the puncture apparatus in the case where the medical tube according to the eleventh embodiment of the present disclosure is applied to the puncture apparatus;

FIGS. 50A and 50B are longitudinal sectional views of a medical tube according to a twelfth embodiment of the present disclosure;

FIGS. 51A and 51B are longitudinal sectional views of a medical tube according to a thirteenth embodiment of the present disclosure;

FIG. 52 is a view (as viewed from the side of the patient's legs) for explaining an operating procedure of a puncture apparatus in the case where a medical tube according to a fourteenth embodiment of the present disclosure is applied to the puncture apparatus;

FIG. 53 is a view (as viewed from the side of the patient's legs) for explaining the operating procedure of the puncture apparatus in the case where the medical tube according to the fourteenth embodiment of the present disclosure is applied to the puncture apparatus;

FIG. 54 is a view for explaining an operating procedure of a puncture apparatus in the case where a medical tube according to a fifteenth embodiment of the present disclosure is applied to the puncture apparatus;

FIG. 55 is a view for explaining an operating procedure of a puncture apparatus in the case where a medical tube according to a sixteenth embodiment of the present disclosure is applied to the puncture apparatus;

FIG. 56 depicts views (a longitudinal sectional view and a cross-sectional view) for explaining an operating procedure of a puncture apparatus in the case where a medical tube according to a seventeenth embodiment of the present disclosure is applied to the puncture apparatus;

FIG. 57 depicts views (a longitudinal sectional view and a cross-sectional view) for explaining an operating procedure of a puncture apparatus in the case where a medical tube according to an eighteenth embodiment of the present disclosure is applied to the puncture apparatus;

FIG. 58 depicts views (a longitudinal sectional view and a cross-sectional view) for explaining an operating procedure of a puncture apparatus in the case where a medical tube according to a nineteenth embodiment of the present disclosure is applied to the puncture apparatus;

FIG. 59 depicts views (a longitudinal sectional view and a cross-sectional view) for explaining an operating procedure of a puncture apparatus in the case where a medical tube according to a twentieth embodiment of the present disclosure is applied to the puncture apparatus;

FIG. 60 depicts views (a longitudinal sectional view and a cross-sectional view) for explaining an operating procedure of a puncture apparatus in the case where a medical tube according to a twenty-first embodiment of the present disclosure is applied to the puncture apparatus;

FIG. 61 depicts views (a longitudinal sectional view and a cross-sectional view) for explaining an operating procedure of a puncture apparatus in the case where a medical tube according to a twenty-second embodiment of the present disclosure is applied to the puncture apparatus;

FIG. 62 depicts views (a longitudinal sectional view and a cross-sectional view) for explaining an operating procedure of a puncture apparatus in the case where a medical tube according to a twenty-third embodiment of the present disclosure is applied to the puncture apparatus;

FIG. 63 depicts a longitudinal sectional view and a cross-sectional view) for explaining an operating procedure of a puncture apparatus in the case where a medical tube according to a twenty-fourth embodiment of the present disclosure is applied to the puncture apparatus;

FIG. 64 depicts a longitudinal sectional view and a cross-sectional view for explaining an operating procedure of a puncture apparatus in the case where a medical tube according to a twenty-fifth embodiment of the present disclosure is applied to the puncture apparatus;

FIG. 65 depicts a longitudinal sectional view and a cross-sectional view for explaining an operating procedure of a puncture apparatus in the case where a medical tube according to a twenty-sixth embodiment of the present disclosure is applied to the puncture apparatus;

FIG. 66 is a perspective view of an implant used with a medical tube according to a twenty-seventh embodiment of the present disclosure;

FIG. 67 is a perspective view of an implant used with a medical tube according to a twenty-eighth embodiment of the present disclosure;

FIG. 68 is a perspective view of an implant used with a medical tube according to a twenty-ninth embodiment of the present disclosure;

FIG. 69 is a perspective view illustrating a medical tube according to a thirtieth embodiment of the present disclosure;

FIG. 70 is a sectional view illustrating a modification of the medical tube depicted in FIG. 69;

FIG. 71 is a sectional view of a puncture apparatus in the case where a medical tube according to a thirty-first embodiment of the present disclosure is applied to the puncture apparatus;

FIG. 72 is a view for explaining an operating procedure of a puncture apparatus in the case where a medical tube according to a thirty-second embodiment of the present disclosure is applied to the puncture apparatus;

FIG. 73 is a view for explaining the operating procedure of the puncture apparatus in the case where the medical tube according to the thirty-second embodiment of the present disclosure is applied to the puncture apparatus; and

FIG. 74 is a view illustrating a condition on the proximal side of a puncture member depicted in FIG. 73.

### DETAILED DESCRIPTION

A medical tube, a medical device set, and an implant indwelling method according to the described aspects of the present disclosure will be described in detail below, with reference to preferred embodiments illustrated in the attached drawings.

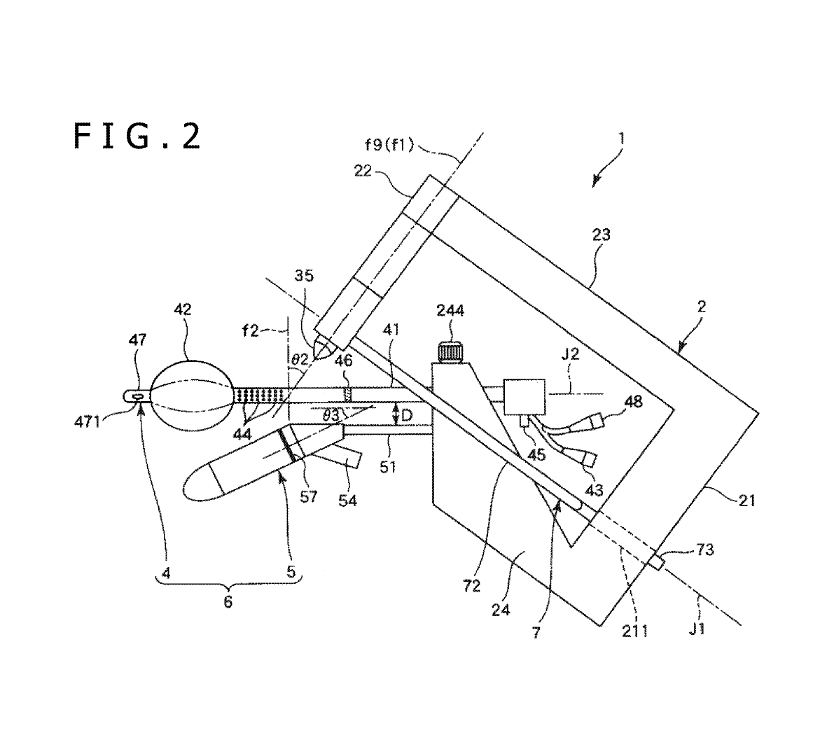

Note that in the following, for convenience of explanation, the left side in FIG. 2 will be referred to as “distal (end),” the right side as “proximal (end),” the upper side as “up,” and the lower side as “down.” FIG. 2 depicts the puncture apparatus in the state of being not yet used, and this state will be referred to also as the “initial state” for convenience of explanation. In addition, a state where a puncture apparatus (insertion tool) is mounted onto a patient will be referred to also as the “mounted state.”

First, a puncture apparatus 1 will be described.

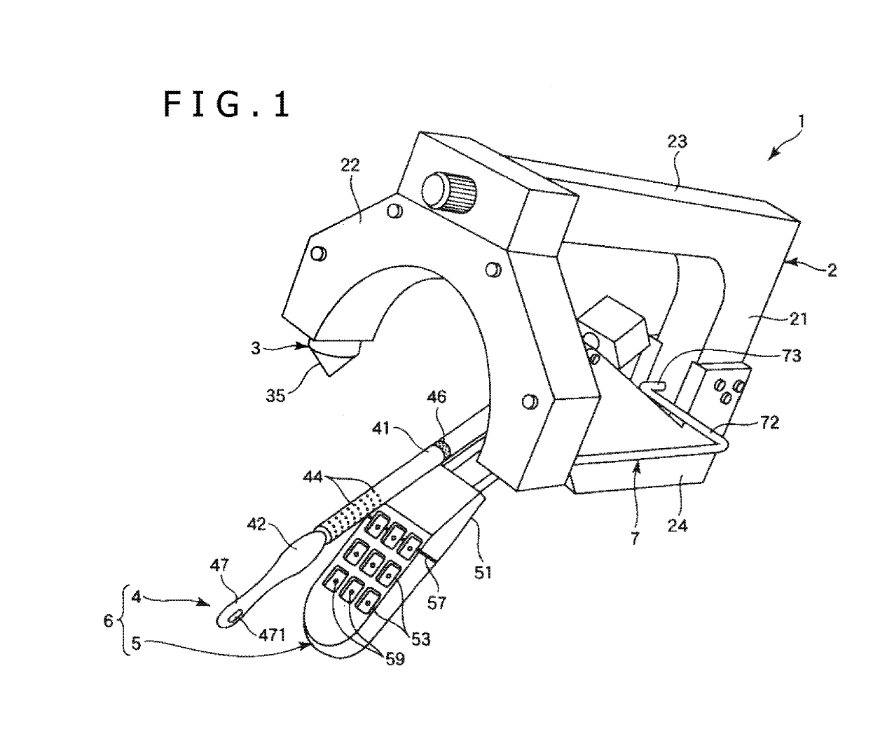

A puncture apparatus 1 depicted in FIGS. 1 and 2 is an apparatus for use in treatment of female urinary incontinence, specifically, for example, in embedding (implanting) in a living body a living body tissue-supporting indwelling article for treatment of urinary incontinence.

The puncture apparatus 1 is a medical device set including a frame (support section) 2, a puncture member 3, a urethral-insertion member 4, a vaginal-insertion member 5, and an operating member 7. The puncture member 3, the urethral-insertion member 4, the vaginal-insertion member 5, and the operating member 7 are supported by the frame 2. In addition, in the puncture apparatus 1, the urethral-insertion member 4 and the vaginal-insertion member 5 constitute an insertion tool 6. These will be sequentially described below.

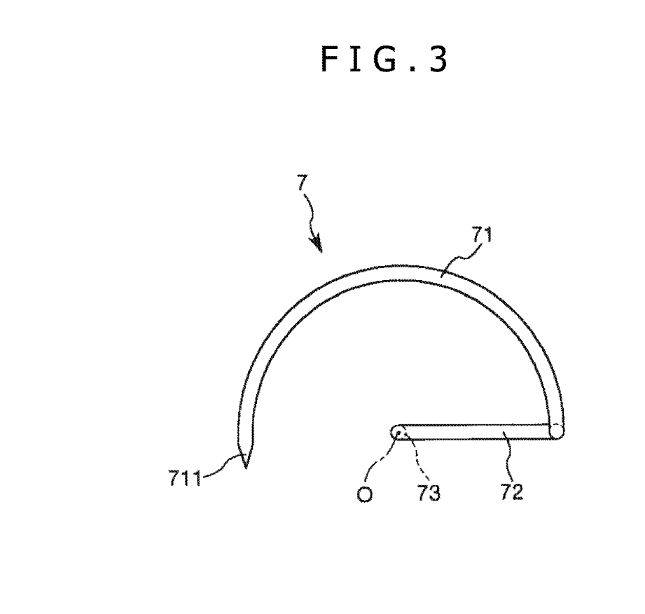

The operating member 7 is a member for operating the puncture member 3. As depicted in FIGS. 1 to 3, such an operating member 7 can include an insertion section 71, a shaft section 73, and an interlock section 72 interlocking the insertion section 71 and the shaft section 73. The insertion member 71, the interlock section 72, and the shaft section 73 may be formed to be integral with one another, or, alternatively, at least one of them may be formed as a separate body from the others of them.

The insertion section 71 is a part to be inserted into the puncture member 3 through a proximal-side opening (proximal-side opening portion) 332, and functions as a stylet for reinforcing the puncture member 3 internally. With the insertion section 71 inserted in the puncture member 3, the puncture member 3 is attached by the operating member 7. As a result, the interlock section 72 interlocked to the insertion section 71 can press the puncture member 3 toward the distal side (other end side), so that an operation of the puncture member 3 by the operating member 7 can be enabled. The insertion section 71 as disclosed above has a circular arc shape corresponding to the shape of the puncture member 3. The center angle of the insertion section 71 is preferably set in accordance with the center angle of the puncture member 3, specifically, for example, set to be equal to the center angle of the puncture member 3. In addition, a distal portion 711 of the insertion section 71 is preferably tapered off. Where the tapered-off distal portion 711 is thus provided, the insertion of the puncture member 3 into the insertion section 71 can be performed relatively smoothly. In addition, with the distal portion 711 tapered off, a needle body 35 present at the distal end of the puncture member 3 can also be reduced in thickness, so that the puncture resistance at the time when the puncture member 3 punctures a living body can be lowered.

The shaft section 73 extends along an axis J1 which intersects a center O of the insertion section 71 and is orthogonal to a plane f1 containing the insertion section 71.

The interlock section 72 interlocks a proximal portion of the insertion section 71 and a distal portion of the shaft section 73. The interlock section 72 has a substantially L-shaped form of being bent substantially at right angle at an intermediate portion thereof. The interlock section 72 can also function as a grasping section to be gripped by an operator at the time of operating the operating member 7.

The operating member 7 as above is configured to be higher in rigidity than a main body (tube main body) 31 of the puncture member 3. The material constituting the operating member 7 is not particularly limited; there can be used, for example, various metallic materials such as stainless steel, aluminum or aluminum alloys, titanium or titanium alloys, etc. In addition, various resin materials can also be used.

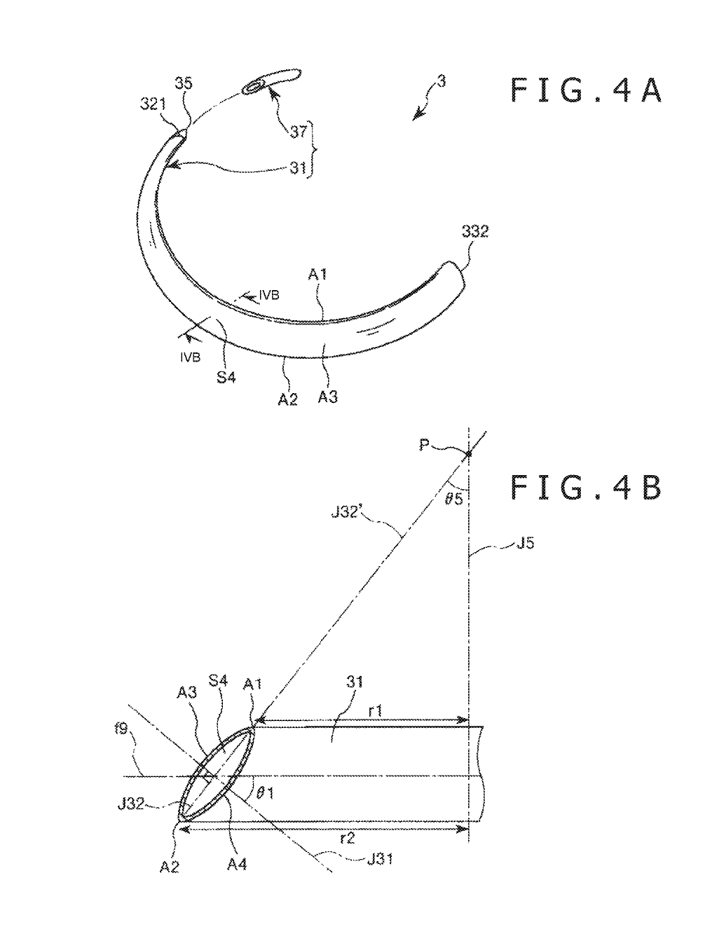

As illustrated in FIG. 4A, the puncture member 3 can include an elongated sheath (medical tube) 30 and the needle body 35 provided at the distal end of the sheath 30, and can be used for puncturing a living body in an assembled state (medical tube assembly) in which the sheath 30 and the needle body 35 are assembled together. The sheath 30 is a member in which an implant main body 91 of an implant 9 is temporarily inserted. The sheath 30 can include a main body 31, which is tubular in shape, and an extension tube 37.

The main body 31 can include an elongated tubular body (tube), opening at both the distal end and the proximal end thereof. Such a main body 31 has an internal space in which an implant main body 91 can be inserted. The main body 31 has a bent shape of being bent in one direction, specifically, for example, in a circular arc shape, and is flat shaped in cross section as depicted in FIG. 4B. In accordance with an exemplary embodiment, for example, the cross-sectional shape at a central portion S4 in the longitudinal direction of the main body 31 is a flat shape including a minor axis J31 and a major axis J32. As will be described later, the implant main body 91 is disposed inside the main body 31. With the main body 31 flat shaped, therefore, the posture of the implant main body 91 within the main body 31 can be controlled.

In addition, the width (the length in the direction of the major axis 32) of the internal space of the main body 31 can be designed to be substantially the same as the width of a main body section 911 (described later) of the implant main body 91 (see FIG. 23), which helps ensure that even when the implant main body 91 is moved, the frictional resistance between the implant main body 91 and the internal space of the main body 31 is low, so that no unnecessary force is exerted on the implant main body 91, and the main body section 911 can be disposed in a sufficiently developed state within the main body 31.

Note that while the flat shape of the main body 31 is an ellipse in the present embodiment, this is not restrictive. For example, convex-like shapes in section, rectangles (flat shapes) with corners rounded, and spindle-like shapes with a central portion enlarged (enlarged in diameter) as compared with both end portions may also be adopted as the flat shape. Where the cross-sectional shape of the insertion section 71 of the operating member 7 inserted in the main body 31 is a flat shape, overlapping of the flat shape of the insertion section 71 with the flat shape of the main body 31 can restrict the insertion section 71 from rotating about an axis thereof in relation to the main body 31.

In the following, for convenience of explanation, an end portion located on the inner side (one end) in the direction of the major axis J32 will be referred to also as an “inner circumferential portion A1,” an end portion located on the outer side (other end) will be referred to also as an “outer circumferential portion A2,” a surface oriented toward the upper side will be referred to also as a “front surface A3,” and a surface oriented toward the lower side will be referred to also as a “back surface A4,” as depicted in FIG. 4B.

As depicted in FIG. 4B, let a plane containing both a center point of the circular arc of the central portion S4 and a center point of the cross-sectional shape with respect to the longitudinal direction of the main body 31 (a plane containing the center axis of the main body 31) be a plane f9, and let the angle formed between the plane f9 and the minor axis J31 at the central portion S4 be an inclination angle θ1, then the inclination angle θ1 is preferably, for example, an acute angle. With the inclination angle θ1 set to be an acute angle, the implant 9 (described later) can be disposed substantially in parallel to the urethra, so that the urethra can be supported more effectively. This effect will be described in detail later.

Note that the inclination angle θ1 is not particularly limited so long as the inclination angle θ1 is an acute angle. Preferably, for example, the inclination angle θ1 is about 20 degrees to 60 degrees, more preferably 30 degrees to 45 degrees, and further preferably about 35 degrees to 40 degrees. This causes a further enhancement of the aforementioned effect.

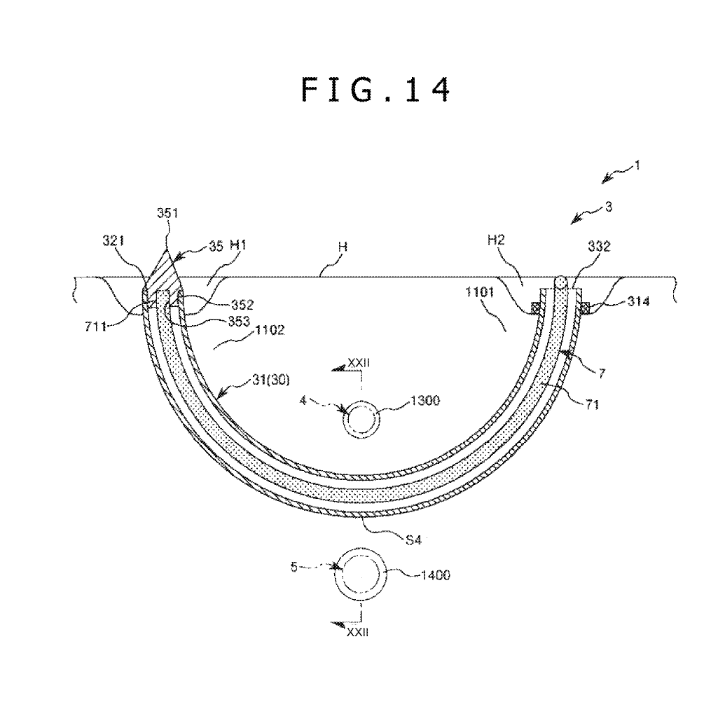

While it is preferable that the inclination angle θ1 satisfies the aforesaid numerical range over the whole region in the extending direction of the main body 31, the aforesaid effect can be exhibited if the aforesaid numerical range is satisfied at least at the central portion S4 in the extending direction of the main body 31. Note that the “central portion S4” means a region including the part located between a urethra 1300 and a vagina 1400 in a state where a living body is punctured by the puncture member 3 (a state where the main body 31 is disposed inside the living body), as illustrated in FIGS. 14 and 16 to 20.

Note that both end portions of the main body 31 may be provided with markers at parts which are located equidistantly from the central portion S4 and which protrude to the outside of a living body in a state where the main body 31 is disposed in the living body, which helps ensure that the position of the central portion S4 inside the living body can be confirmed by comparing the positions of both the markers.

The configuration of the main body 31 can be described in other words as follows. It can be also said that as depicted in FIG. 4B, the main body 31 is so formed that the major axis J32 is inclined against a center axis J5 of the circular arc and that the center axis J5 of the circular arc and an extension line J32′ of the major axis J32 have an intersection P. In this case, an angle θ5 formed between the center axis J5 and the extension line J32′ is equal to the inclination angle θ1. In accordance with an exemplary embodiment, it can be also said that, in plan view as viewed from the direction of the center axis J5 of the main body 31, the main body 31 has the inner circumferential portion A1 located at its inner circumferential edge and having a minimum radius of curvature r1 and the outer circumferential portion A2 located at its outer circumferential edge and having a maximum radius of curvature r2, and, as depicted in FIG. 4B, the inner circumferential portion A1 and the outer circumferential portion A2 are located to be spaced from each other in the direction of the center axis J5.

In accordance with an exemplary embodiment, since the main body 31 is flat in shape and less liable to crush in the major axis direction, the separated distance between the inner circumferential portion A1 and the outer circumferential portion A2 is less likely to vary. In addition, the inner circumferential portion A1 and the outer circumferential portion A2 can be larger in curvature and less liable to deform than that of the front surface A3 and the back surface A4. For this reason, a main body section 911 of the implant main body 91 inserted in the main body 31 can be securely prevented from being unintendedly deformed.

As illustrated in FIG. 14, at a proximal portion of the main body 31, there is provided a flange portion 314 as an attachment member attaching a body surface (living body surface) H. The flange portion 314 is a ring shaped portion having an outside diameter greater than the outside diameter of the main body 31. Note that the flange portion 314 may be formed integrally with the main body 31, or may be configured as a separate body from the main body 31 and fixed to the main body 31.

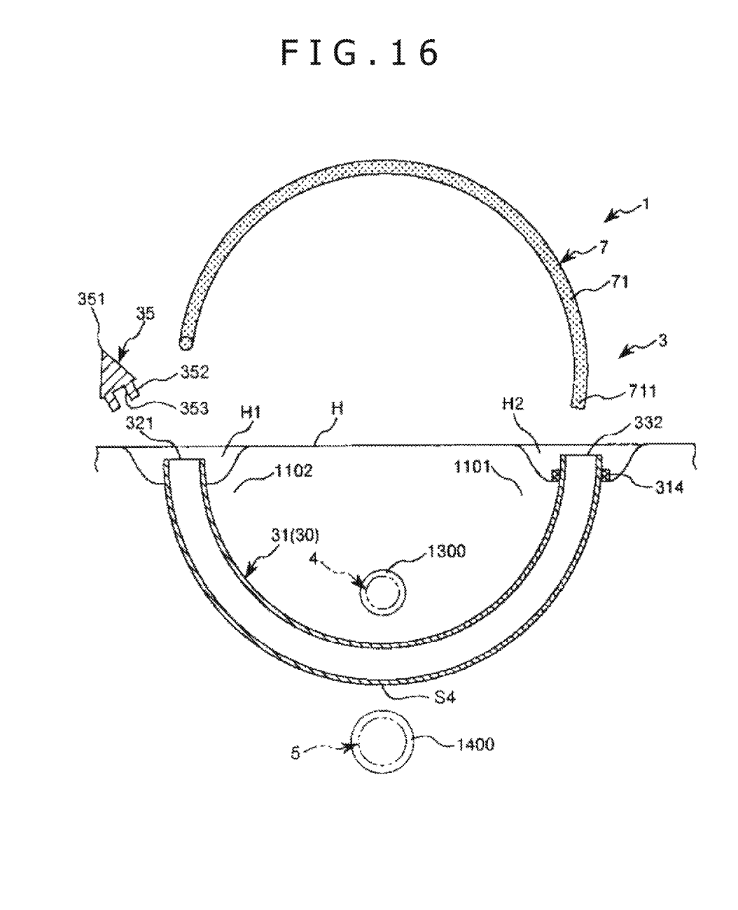

As the puncture member 3 punctures a living body, the flange portion 314 attaches the body surface H and, thereafter, can further press the body surface H. By this pressing, the flange portion 314 can press down the body surface H in the surroundings. As a result, the proximal-side opening 332 of the main body 31 can be prevented from being embedded in the living body to be thereby hidden. Accordingly, an operation of drawing the insertion section 71 of the insertion member 7 out of the main body 31 can be performed relatively easily, as depicted in FIG. 16.

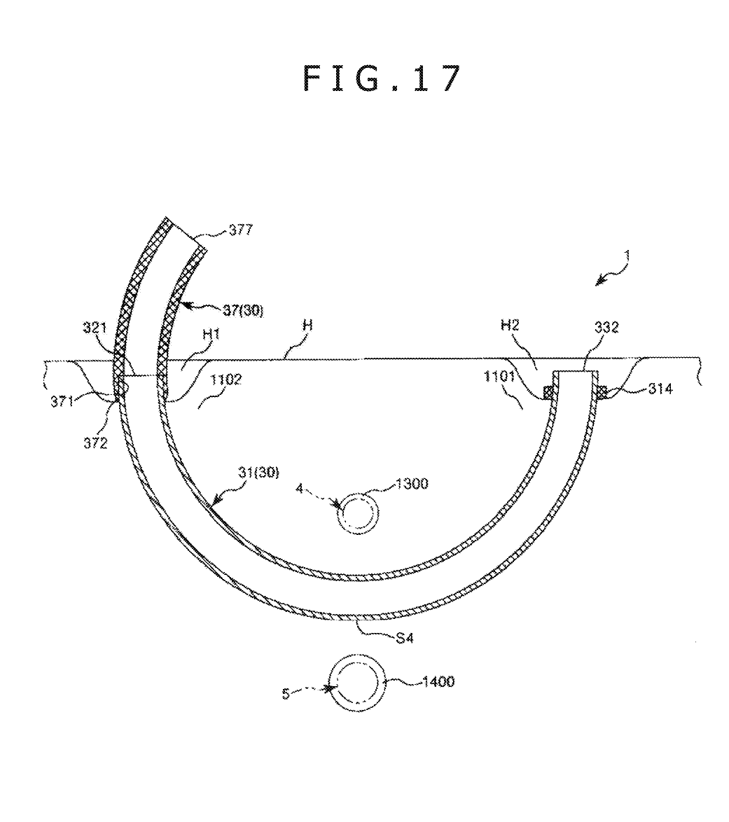

In accordance with an exemplary embodiment, on the distal side of the main body 31, the needle body 35 is mounted on a distal-side opening (distal-side opening portion) 321 in the state depicted in FIG. 14, and this needle body 35 is freely detachable from the distal-side opening 321 as depicted in FIG. 16. After the needle body 35 is detached, the extension tube 37 can be connected to a distal portion of the main body 31, as illustrated in FIG. 17. This connection results in a state where the overall length of the sheath 30 is extended by a length corresponding to the extension tube 37.

The extension tube 37 is composed of a tube shaped member, whose cross-sectional shape is a flat shape like the cross-sectional shape of the main body 31, as depicted in FIG. 25.

At a proximal inner circumferential portion of the extension tube 37, there can be provided an enlarged diameter portion 371 enlarged in inside diameter. In connecting the extension tube 37 to the main body 31, a distal portion of the main body 31 can be fitted to the extension tube 37 from the inner side of the enlarged diameter portion 371, which results in a state where the main body 31 and the extension tube 37 are connected, and communicating, with each other. This state will hereinafter be referred to as the “extension tube connected state.”

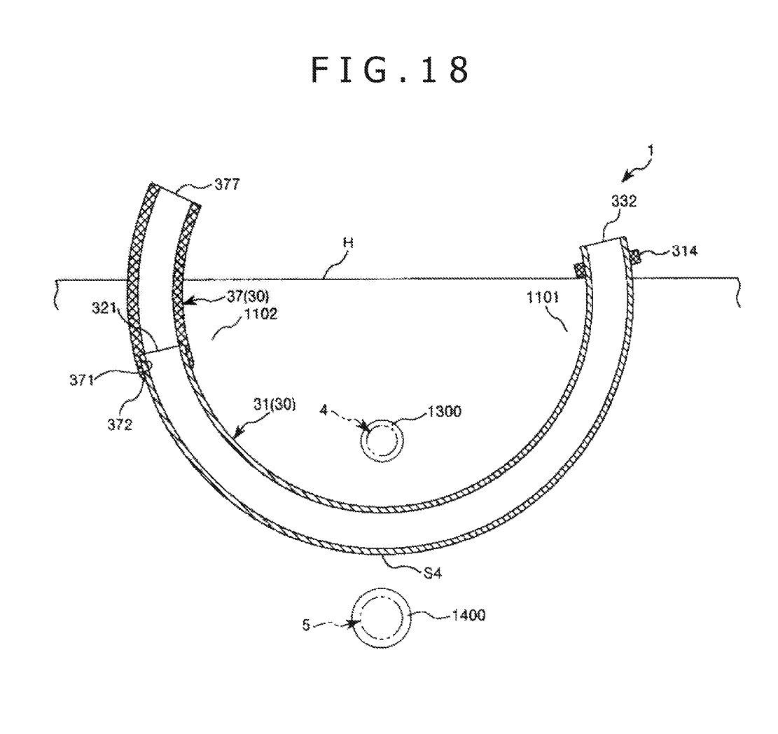

At a proximal outer circumferential portion of the extension tube 37, there is provided a tapered portion 372 gradually decreasing in outside diameter along the proximal direction. This tapered portion 372 can help ensure that, in the extension tube connected state that a step portion that protrudes radially outward is not formed at the boundary between the main body 31 and the extension tube 37. Meanwhile, as depicted in FIGS. 17 and 18, a proximal portion of the extension tube 37 in the extension tube connected state may be embedded in the living body. In this embedding process, the living body tissue can easily come over the tapered portion 372. As a result, the living body tissue can be prevented from being caught on the boundary between the main body 31 and the extension tube 37. Accordingly, the living body tissue can be prevented from being damaged due to the catching.

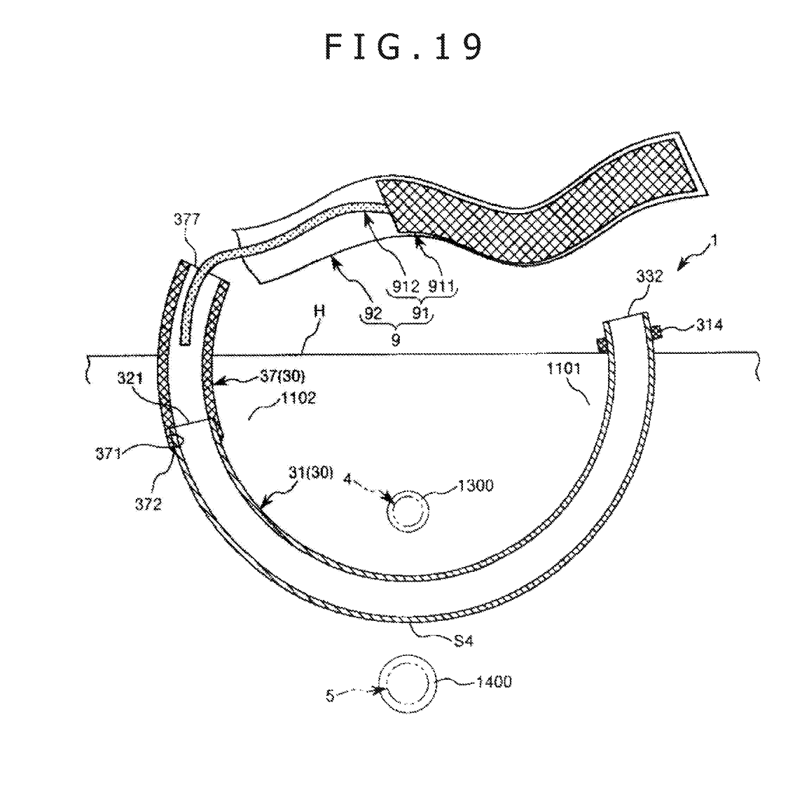

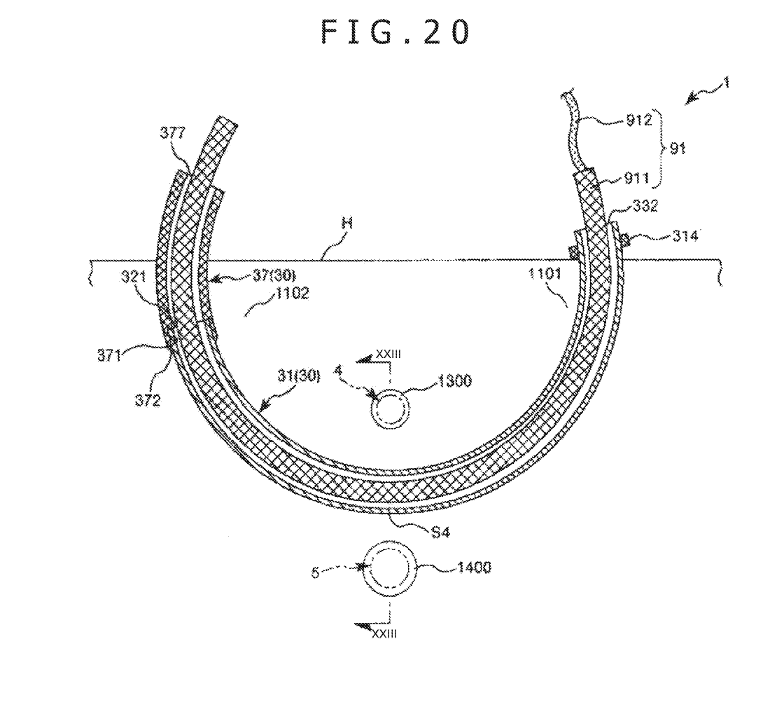

In addition, as depicted in FIGS. 19 and 20, in the extension tube connected state, an operation of inserting and passing the implant main body 91 into and through the sheath 30 can be performed relatively quickly. In addition, there is an advantage that an operation of drawing out the sheath 30 is relatively easy to carry out.

In accordance with an exemplary embodiment, the extension tube 37 can be curved in a circular arc shape with the same curvature as the main body 31. As a result, the sheath 30 is curved in a circular arc shape also as a whole in the extension tube connected state, so that the operation of inserting and passing the implant main body 91 into and through the sheath 30 can be performed relatively easily and swiftly. In addition, since the extension tube 37 and the main body 31 have the same curvature, there are merits that they can be easily rotated in a living body, their centers can be matched to each other easily, and the subsequent procedure can be carried out relatively quickly.

Note that the sheath 30 is curved in a circular arc shape as a whole in the extension tube connected state in the present embodiment, this is not restrictive. It is sufficient that at least a central portion S4 of the main body 31 is curved in a circular arc shape.

In accordance with an exemplary embodiment, while the extension tube 37 is connected to a distal portion of the main body 31 in the present embodiment, this is not restrictive. The extension tube 37 may be connected to a proximal portion of the main body 31, or may be connected to both end portions of the main body 31.

In addition, the extension tube 37 may be so configured that the extension tube 37 itself can be extended and contracted, and its deformed state is maintained.

Meanwhile, as illustrated in FIG. 14, the puncture member 3 punctures the body surface H in the vicinity of an inguinal region on one side of the patient to enter the body, then sequentially passes an obturator foramen 1101 on the one side, between the urethra 1300 and the vagina 1400, and an obturator foramen 1102 on the other side, and thereafter protrudes to the outside of the body via the body surface H in the vicinity of an inguinal region on the other side, whereby a puncture hole can be formed.

However, depending on the patient's body type, for example, in the case of a comparatively large patient, if the extension tube 37 is not used but only the main body 31 is left in the living body solely, the main body 31 would be embedded in the living body due to a restoring force of the living body itself. Insertion of the implant main body 91 into the main body 31 thus embedded in the living body can be extremely difficult to carry out.

In the puncture apparatus 1, however, the extension tube connected state can be obtained using the extension tube 37, so that both end portions of the sheath 30 in the extension tube connected state can be protruded from the body surface H, as depicted in FIG. 18. Consequently, as illustrated in FIG. 19, an operation of inserting the implant main body 91 into the sheath 30 can be carried out relatively easily and assuredly.

As has been described above, the needle body 35 is provided at the distal end of the main body 31. As illustrated in FIGS. 14 and 16, the needle body 35 can include a tapered sharp needle tip 351 and a proximal section 352 provided on the proximal side of the needle tip 351. The proximal section 352 is inserted into the main body 31, whereby the needle body 35 is retained on the main body 31 in a freely detachable manner. Note that the needle tip 351 protrudes from the distal-side opening 321 of the main body 31 in a state where the proximal section 352 is inserted in the main body 31, which helps enable reliable puncturing of a living body. In addition, the proximal section 352 is fitted in the main body 31 with such a force that unintended detachment of the needle body 35 from the main body 31 can be prevented from occurring.

In addition, the proximal section 352 is provided with an engaging section 353 for engagement with the distal portion 711 of the insertion section 71. The engaging section 353 can include a recess, and, in an inserted state where the puncture member 3 is inserted in the insertion section 71, the distal portion 711 is located inside the engaging section 353. With the engaging section 353 provided, displacement of the needle body 35 relative to the insertion section 71 is restrained, and puncture of a living body by the puncture member 3 can be performed with enhanced smoothness.

Note that the center angle of the puncture member 3 having the circular arc shape is not particularly limited, and is appropriately set according to various conditions. As will be described later, the center angle is so set that the needle body 35 can enter a patient's body via an inguinal region on one side of the patient, pass between the urethra 1300 and the vagina 1400, and protrudes to the outside of the body via an inguinal region on the other side. Specifically, for example, the center angle is preferably 150 degrees to 270 degrees, more preferably 170 degrees to 250 degrees, and further preferably 190 degrees to 230 degrees.

The materials constituting the sheath 30 and the needle body 35 are preferably, for example, rigid materials such as to maintain the shape of the puncture member 3 and the internal space in a state where the puncture member 3 is inserted in a living body. Examples of such rigid materials applicable here can include various resin materials such as polyethylene, polyimides, polyamides, polyester elastomers, polypropylene, etc. and various metallic materials such as stainless steel, aluminum or aluminum alloys, titanium or titanium alloys, etc. Note that the sheath 30 and the needle body 35 may not necessarily be configured by adopting rigid materials, but may be configured by use of other materials than rigid materials; in the latter case, the wall of the main body 31 may be reinforced with a reinforcement member. For example, a braiding with high strength may be embedded in the wall, whereby the shape of the puncture member 3 and the internal space can be maintained in the state where the puncture member 3 is inserted in a living body. Another example of the reinforcement member is a spiral body, which is embedded in the wall of the main body 31, whereby flexibility can be ensured while the internal space is retained to such an extent that an inserted article can be slid therein.

In accordance with an exemplary embodiment, the sheath 30 is preferably light-transmitting so that the inside of the sheath 30 can be visually checked externally, which helps make it possible, for example, to check whether the distal portion 711 of the insertion section 71 inserted in the sheath 30 is in engagement with the engaging section 353.

The frame 2 retains the operating member 7 with the puncture member 3 mounted thereto so that the operating member 7 is rotationally movable, and fixes the insertion tool 6 in a freely detachable manner. The frame 2 has a function of determining a puncture path of the needle body 35 when the puncture member 3 punctures the living body tissue. Specifically, the frame 2 determines the positional relations of the puncture member 3, the urethral-insertion member 4, and the vaginal-insertion member 5 so that the needle body 35 passes between the urethral-insertion member 4 and the vaginal-insertion member 5 without colliding against any of these insertion members when the puncture member 3 punctures the living body tissue.

As depicted in FIGS. 1 and 2, the frame 2 can include a bearing section 21 for bearing the shaft section 73 of the operating member 7, a guide section (retaining section) 22 for guiding the puncture member 3, an interlock section 23 interlocking the bearing section 21 and the guide section 22, and a fixing section 24 to which the insertion tool 6 is fixed.

The bearing section 21 is located on the proximal side of the puncture apparatus 1, and extends in a direction substantially orthogonal to the axis J1. The bearing section 21 is formed with a through-hole 211 on the axis J1, and the shaft section 73 is inserted in the through-hole 211 in a rotationally movable manner. As a result, the operating member 7 is supported on the frame 2 so as to be rotationally movable about the axis J1.

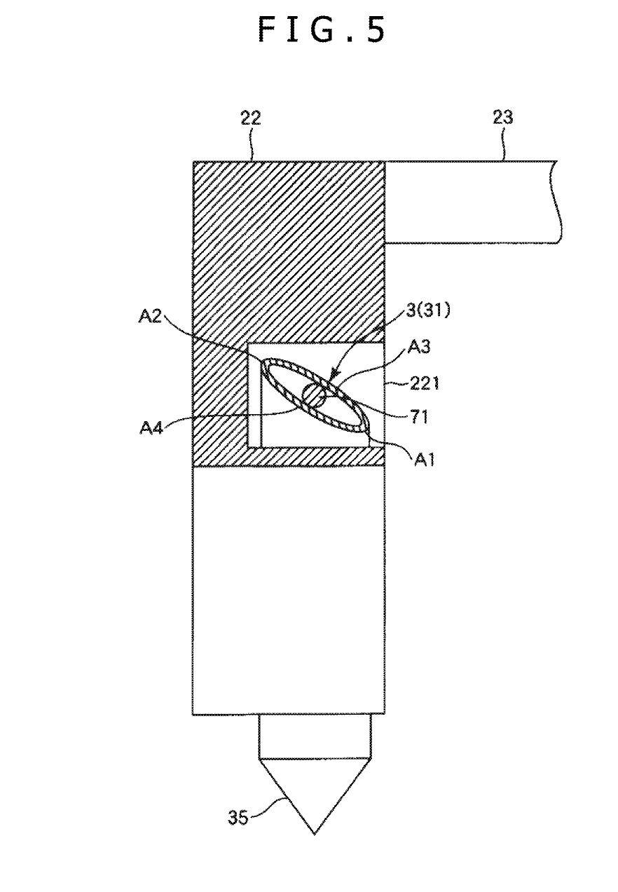

The guide section 22 is located on the distal side of the puncture apparatus 1, and is disposed opposite to the bearing section 21. The guide section 22 can be formed therein with a roughly C-shaped guide groove 221 for accommodating the puncture member 3 and guiding the puncture member 3. In addition, as depicted in FIG. 5, in a state of being disposed within the guide groove 221, the puncture member 3 has its back surface A4 located on the distal side and has its front surface A3 located on the proximal side.

In the initial state depicted in FIGS. 1 and 2, a rotating operation on the operating member 7 causes the puncture member 3 to gradually protrude from the guide section 22 so as to puncture a living body.

The interlock section 23 interlocks the bearing section 21 and the guide section 22. In addition, the interlock section 23 has a rod-like shape extending substantially in parallel to the axis J1. The interlock section 23 can also function as a grasping section, and an operator can use the puncture apparatus 1 by gripping the interlock section 23.

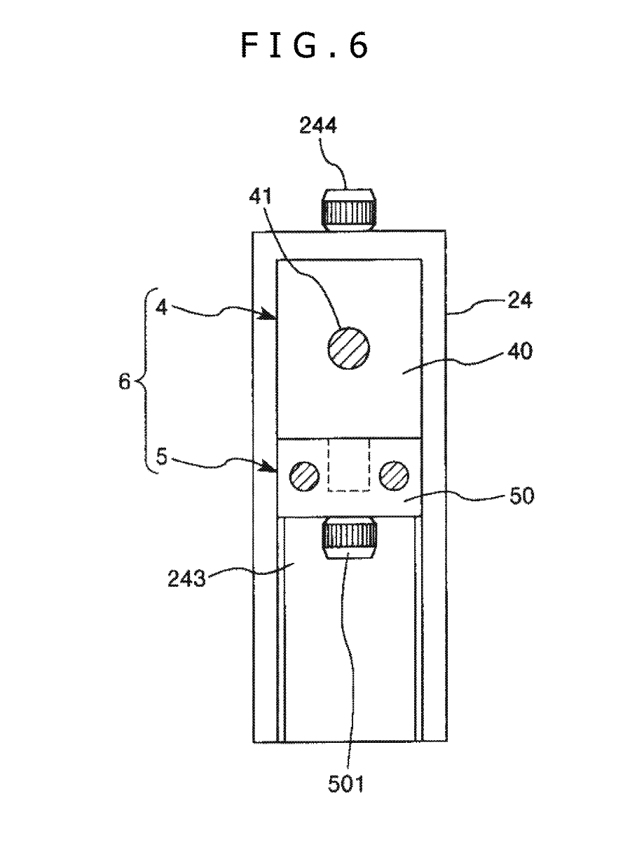

The fixing section 24 is disposed opposite to the interlock section 23, with the axis J1 interposed therebetween. As depicted in FIG. 6, the fixing section 24 is provided with a recess 243 in which to fit a support section 60 (described later) of the insertion tool 6, and a male screw 244. With a support section 60 fitted into the recess 243 and with the male screw 244 fastened into a female screw (not illustrated) of the support section 50, the insertion tool 6 can be fixed to the fixing section 24.

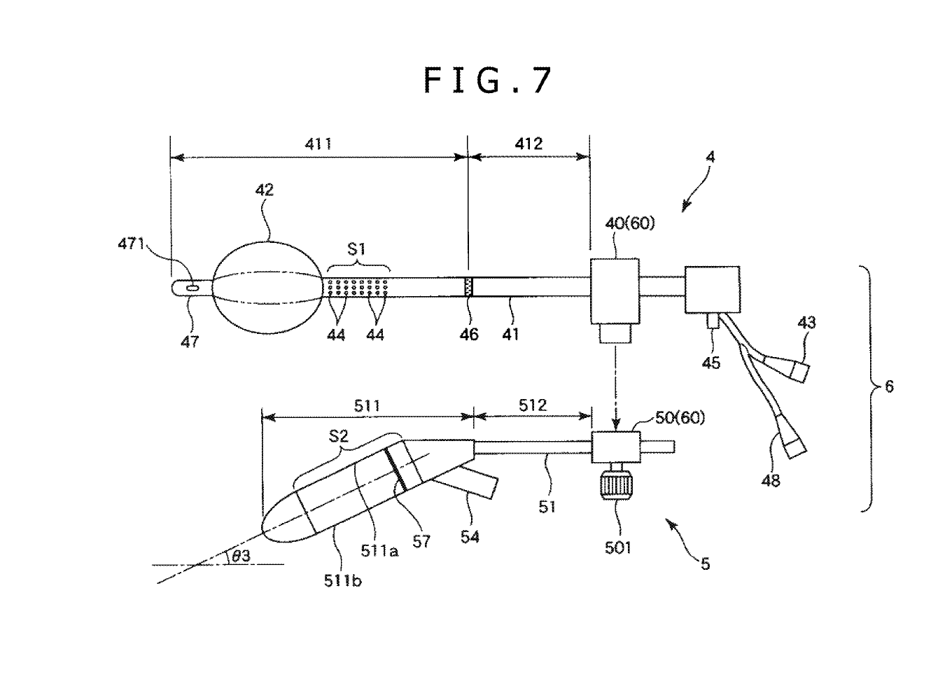

As illustrated in FIGS. 1 and 7, the insertion tool 6 can include a urethral-insertion section (second insertion section) 41 to be inserted into a urethra 1300, a vaginal-insertion section (first insertion section) 51 to be inserted into a vagina 1400, and the support section 60 supporting the urethral-insertion section 41 and the vaginal-insertion section 51. As aforementioned, the insertion tool 6 can include the urethral-insertion member 4 and the vaginal-insertion member 5, wherein the urethral-insertion member 4 has the urethral-insertion section 41, and the vaginal-insertion member 5 has the vaginal-insertion section 51. The support section 60 can include a support section 40, which is possessed by the urethral-insertion member 4 and which supports the urethral-insertion section 41, and a support section 50, which is possessed by the vaginal-insertion member 5 and which supports the vaginal-insertion section 51. In the insertion tool 6, the urethral-insertion member 4 and the vaginal-insertion member 5 is freely detachable each other through the support sections 40 and 50. The urethral-insertion member 4 and the vaginal-insertion member 5 will be sequentially described below.

The urethral-insertion member 4 can include the urethral-insertion section 41 which is elongated and which, from its distal end to its intermediate portion, is to be inserted into the urethra 1300, and the support section 40 supporting the urethral-insertion section 41. Note that in the following, for convenience of explanation, the part located inside the urethra 1300 (inclusive of a bladder 1310) in the mounted state will be referred to also as the “insertion section 411,” whereas the part exposed from the urethral orifice to the outside of the body in the mounted state will be referred to also as the “non-insertion section 412.”

In accordance with an exemplary embodiment, the urethral-insertion section 41 has a straight tubular shape with the distal end rounded. The insertion section 411 is provided at a distal portion thereof with an expandable and contractible balloon 42, and a urine drain section 47. The balloon 42 can function as a restriction unit, which restricts the position in the axial direction of the urethral-insertion member 4 within the urethra 1300. Specifically, at the time of using the puncture apparatus 1, the balloon 42 is expanded after inserted into a patient's bladder 1310. Then, the expanded balloon 42 is caught on a bladder neck, whereby the position of the urethral-insertion member 4 relative to the bladder 1310 and the urethra 1300 is fixed. In accordance with an exemplary embodiment, the urine drain section 47 can be used for draining urine present in the bladder 1310.

The balloon 42 is connected to a balloon port 43 provided at a proximal portion of the urethral-insertion section 41, through the inside of the urethral-insertion section 41. A balloon expanding device such as a syringe can be connected to the balloon port 43. The balloon 42 is expanded when a working fluid (a liquid such as physiological saline solution, or a gas or the like) is supplied from the balloon expanding device into the balloon 42. In contrast, the balloon 42 is contracted when the working fluid is drawn out of the balloon 42 by the balloon expanding device. Note that in FIG. 7, the contracted state of the balloon 42 is indicated by alternate long and two short dashes line, and the expanded state of the balloon 42 is indicated by solid line.

In accordance with an exemplary embodiment, the urine drain section 47 is provided with a drain hole 471 through which the inside and the outside of the urine drain section 47 communicate with each other. The urine drain section 47 is connected to a urine drain port 48 provided at a proximal portion of the urethral-insertion section 41, through the inside of the urethral-insertion section 41. Therefore, urine introduced via the drain hole 471 can be drained via the urine drain port 48.

The balloon 42 and the urine drain section 47 can be configured, for example, by a double lumen.

The insertion section 411 is formed at an intermediate portion thereof with a plurality of suction holes 44. The plurality of suction holes 44 can be disposed over the whole circumferential range of the urethral-insertion section 41. Each of the suction holes 44 is connected to a suction port 45 provided at a proximal portion of the urethral-insertion section 41, through the urethral-insertion section 41. A suction device such as a pump can be connected to the suction port 45. When the suction device is operated in a state where the urethral-insertion section 41 is inserted in the urethra 1300, a urethral wall can be secured by suction onto the urethral-insertion section 41. When in this condition the urethral-insertion section 41 is pushed in toward the distal side (into the body), the urethra 1300 is also pushed in attendantly, whereby it is possible, for example, to shift the bladder 1310 to such a position as not to overlap with a puncture path for the puncture member 3, and thereby to secure the puncture path for the puncture member 3. Accordingly, puncture by the puncture member 3 can be performed relatively accurately and safely. Note that the number of the suction holes 44 is not particularly limited; for example, only one suction hole may be provided. In addition, the layout of the suction holes 44 is not specifically restricted; for example, the suction holes 44 may be formed in only part in the circumferential direction of the urethral-insertion section 41.

At the boundary between the insertion section 411 and the non-insertion section 412, a marker 46 can be provided for confirming the depth of insertion of the urethral-insertion section 41 into the urethra 1300. The marker 46 is located at the urethral orifice when the urethral-insertion section 41 is inserted in the urethra 1300 and the balloon 42 is located inside the bladder 1310. As a result, the depth of insertion of the insertion section 411 into the urethra 1300 can be confirmed relatively easily. In accordance with an exemplary embodiment, It can be sufficient for the marker 46 to be visibly checkable externally; thus, the marker 46 may be configured, for example, as a colored part, a rugged part or the like. Note that graduations indicative of the distance from the distal end of the urethral-insertion section 41 may be provided in place of the marker 46.

The length of the insertion section 411 is not particularly limited, and may be appropriately set according to the length of the patient's urethra 1300, the shape of the patient's bladder 1310, etc. In view of that the length of a female urethra is generally about 30 mm to 50 mm, it is preferred that the length of the insertion section 411 is about 50 mm to 100 mm.

The length of the non-insertion section 412 (the separated distance between the urethral orifice and the support section 40) is not specifically restricted, and is preferably, for example, not more than about 100 mm, more preferably in the range of about 20 mm to 50 mm. By this, the non-insertion section 412 can be made to have a suitable length, and operability can be enhanced. In accordance with an exemplary embodiment, for example, if the length of the non-insertion section 412 exceeds the aforesaid upper limit, there may arise, depending on the configuration of the frame 2 or the like, a situation in which the center of gravity of the puncture apparatus 1 is largely spaced from the patient and, accordingly, the stability of the puncture apparatus 1 in the mounted state may be lowered.

The material constituting the urethral-insertion member 4 is not particularly limited. For example, various metallic materials such as stainless steel, aluminum or aluminum alloys, titanium or titanium alloys, etc. and various resin materials can be used.

Here, an inclination angle θ2 of the plane f9 (plane f1) against a plane f2 orthogonal to the axis J2 of the urethral-insertion section 41 is preferably about 20 degrees to 60 degrees, more preferably about 30 degrees to 45 degrees, and further preferably about 35 degrees to 40 degrees. In other words, the main body 31 is preferably so set indwelling in a body that the angle formed between the plane f9 and the plane orthogonal to the axis of the urethra 1300 is about 20 degrees to 60 degrees, more preferably so set indwelling in the body that the angle is about 30 degrees to 45 degrees, and further preferably so set indwelling in the body that the angle is about 35 degrees to 40 degrees, which helps ensure that puncture by the puncture member 3 can be performed relatively easily, and the puncture distance in puncture by the puncture member 3 can be made shorter.

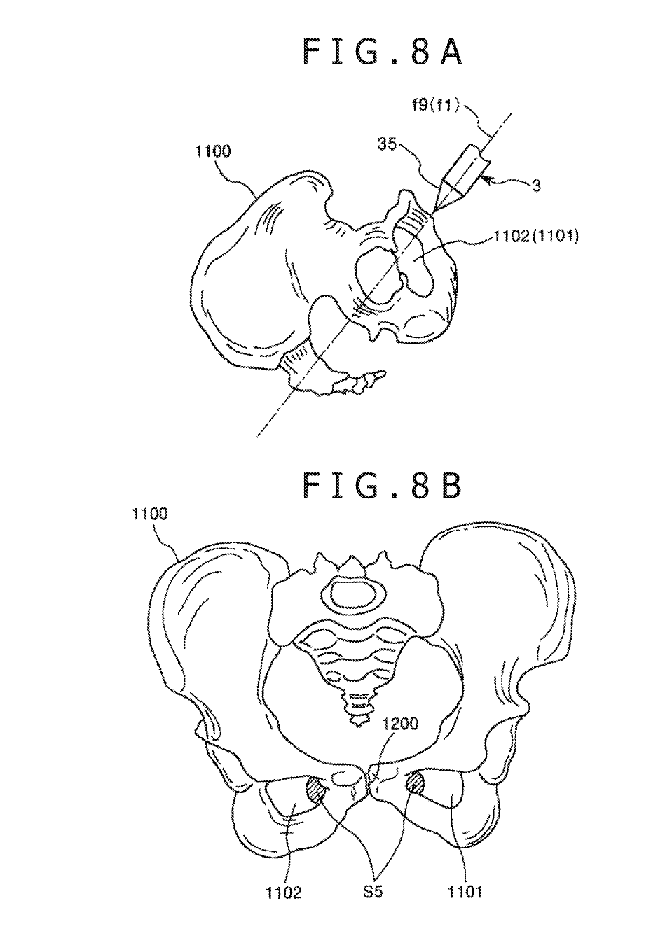

Describing more specifically, with the inclination angle θ2 set within the aforesaid range, the puncture member 3 can capture left and right obturator foramens 1101 and 1102 of a pelvis 1100 wider on a planar basis, as depicted in FIG. 8A, and a wide puncture space for the puncture member 3 can be secured. In accordance with an exemplary embodiment, in a state where a patient is set in a predetermined position (lithotomy position), the puncture member 3 can be made to puncture in a direction comparatively nearer to a perpendicular direction relative to the obturator foramens 1101 and 1102. Therefore, the puncture by the puncture member 3 can be carried out relatively easily. In addition, where the puncture member 3 is made to puncture in a direction comparatively nearer to the perpendicular direction relative to the obturator foramens 1101 and 1102, the needle body 35 of the puncture member 3 passes a shallow portion of the tissue, so that the needle body 35 of the puncture member 3 can pass between the left and right obturator foramens 1101 and 1102 while taking a shorter course. Therefore, as depicted in FIG. 8B, the puncture member 3 can be made to pass those zones in the obturator foramens 1101 and 1102 which are near a pubic symphysis 1200, preferably, safety zones S5. Since the safety zones S5 are parts where there are few nerves and blood vessels, which should be prevented from being damaged, the puncture member 3 can be made to puncture relatively safely. Accordingly, a less invasive procedure can be realized, and the burden on the patient can be reduced to a relatively low level. Thus, with the inclination angle θ2 set within the aforesaid range, the puncture of the patient by the puncture member 3 can be suitably performed. In addition, the puncture at the aforementioned angle makes it relatively easier to aim at the tissue between a middle-part urethra (which refers to a middle part in the longitudinal direction of the urethra 1300) and the vagina 1400. The position between the middle-part urethra and the vagina 1400 is a position suitable as a part where to perform treatment of urinary incontinence by embedding the implant 9.

In accordance with an exemplary embodiment, where the inclination angle θ2 is below the aforesaid lower limit or above the aforesaid upper limit, there may arise, depending on individual differences concerning the patient or the posture of the patient during the procedure or the like, a situation where the puncture member 3 cannot capture the obturator foramens 1101 and 1102 wide on a planar basis or where the puncture path cannot be made sufficiently short.

More preferably, for example, the puncture is conducted in a state where the urethra 1300 or the vagina 1400 or both the urethra 1300 and the vagina 1400 are positionally shifted in the manner of being pushed in toward the inner side of the body, whereby a region between the middle-part urethra and the vagina 1400 can be punctured relatively easily. The method for pushing in either one of the urethra 1300 and the vagina 1400 toward the inner side of the body can, for example, be a method wherein the urethral-insertion member 4 and/or the vaginal-insertion member 5 is inserted into a suitable position, then, in this condition, the urethra 1300 and/or the vagina 1400 is attracted by suction by the suction holes 44 and 59 (described later) provided in these insertion members, and thereafter the urethral-insertion member 4 and/or the vaginal-insertion member 5 is moved further toward the inner side of the body along the axis thereof to a predetermined position. Where the puncture is conducted by setting the main body 31 perpendicularly relative to the left and right obturator foramens 1101 and 1102 of the pelvis in the state where at least one of the urethra 1300 and the vagina 1400 has thus been positionally shifted in the manner of being pushed in toward the inner side of the body, a passage can be formed in a position suitable for indwelling of the implant 9.

In accordance with an exemplary embodiment, It can be preferable to form the passage by adopting a setting such that the trajectory of the main body 31 passes the safety zones S5 in the left and right obturator foramens 1101 and 1102 of the pelvis, shifting at least one of the urethra 1300 and the vagina 1400 toward the inner side of the body so that the trajectory is positioned between the middle-part urethra and the vagina 1400, and performing the puncture by the main body 31 along the trajectory.

As depicted in FIGS. 1 and 7, the vaginal-insertion member 5 can include the vaginal-insertion section (first insertion section) 51 which is elongated and which, from its distal end to its intermediate portion, is to be inserted in the vagina 1400, and the support section 50 which supports the vaginal-insertion section 51. Note that in the following, for convenience of explanation, the part located inside the vagina 1400 in the mounted state will be referred to also as the “insertion section 511,” whereas the part which is exposed from the vaginal orifice to the outside of the body in the mounted state and which ranges to the support section 50 will be referred to also as the “non-insertion section 512.”

In accordance with an exemplary embodiment, the insertion section 511 is elongated in shape. The insertion section 511 extends while being inclined against the insertion section 411 so as to be spaced away from the insertion section 411 on the distal side. With the insertion section 511 inclined against the insertion section 411, the positional relation between the insertion sections 411 and 511 can be made close to the positional relation between the urethra 1300 and the vagina 1400, as compared with the case where the insertion section 511 is not inclined. Therefore, in the mounted state, the puncture apparatus 1 can be held onto the patient more stably, and the burden on the patient can be alleviated. An inclination angle θ3 of the insertion section 511 against the insertion section 411 is not particularly limited; for example, the inclination angle θ3 is preferably, for example, about 0 degree to 45 degrees, more preferably about 0 degree to 30 degrees, which helps enable the aforesaid effects to be exhibited more remarkably. In accordance with an exemplary embodiment, if the inclination angle θ3 is below the aforesaid lower limit or above the aforesaid upper limit, there may arise, depending on individual differences concerning the patient or the posture of the patient during the procedure or the like, a situation in which the vagina 1400 or the urethra 1300 is unnaturally deformed in the mounted state, and the puncture apparatus 1 is not held stably.

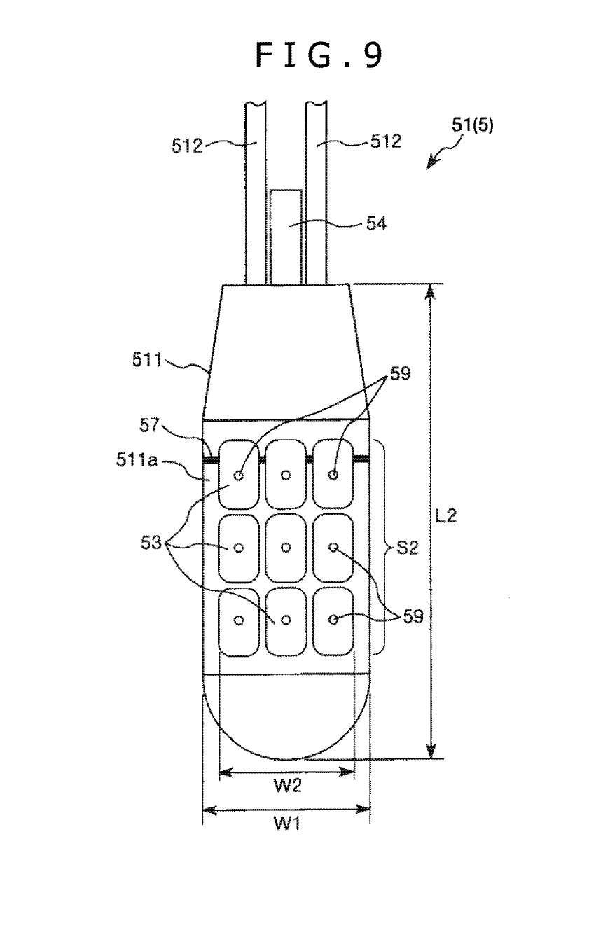

As depicted in FIG. 9, the insertion section 511 can have a flat shape crushed in the vertical direction of the puncture apparatus 1 (in the direction in which the urethra 1300 and the vagina 1400 are arrayed). In addition, the insertion section 511 can include a central portion which is substantially constant in width, and a distal portion which is somewhat rounded. A length L2 of the insertion section 511 is not particularly limited, and is preferably, for example, about 20 mm to 100 mm, more preferably about 30 mm to 60 mm. In addition, a width W1 is not specifically restricted, and is preferably, for example, about 10 mm to 50 mm, more preferably about 20 mm to 40 mm. Further, the thickness of the insertion section 511 is not particularly limited, and is preferably, for example, about 5 mm to 25 mm, more preferably about 10 mm to 20 mm. With the length, width, and thickness set in these ranges, the insertion section 511 can be made to have a shape and a size suited to most vaginas 1400. Accordingly, the stability of the puncture apparatus 1 in the mounted state can be increased, and the burden on the patient can be lessened.

An upper surface (a surface on the urethral-insertion section 41 side) 511a of the insertion section 511 is formed with a plurality of bottomed recesses 53. Note that the number of the recesses 53 is not particularly limited, and, for example, only one recess may be provided. At a bottom surface of each recess 53, there is provided a single suction hole 59. Each suction hole 59 is connected to a suction port 54 provided at a proximal portion of the insertion section 511, by way of the inside of the insertion section 511. The suction port 54 is so provided as to be located outside the living body in the mounted state. A suction device such as a pump can be connected to the suction port 54. When the suction device is operated in a state where the insertion section 511 is inserted in the vagina 1400, a vaginal anterior wall 1410 which is an upper surface of the vaginal wall can be secured by suction onto the insertion section 511. When the vaginal-insertion section 51 is pushed in toward the distal side (into the body) with the vaginal wall thus fixed by suction, the vaginal wall can be pushed in attendantly. Therefore, the disposition and shape of the vaginal wall can be conditioned, a puncture path for the puncture member 3 can be secured, and puncture by the puncture member 3 can be carried out relatively accurately and safely.

A region S2 where the plurality of recesses 53 are formed is disposed to face a region S1. The needle tip of the puncture member 3 passes between these regions S1 and S2. As aforementioned, since a urethral wall (which is a lower surface of the urethra 1300) is suction held onto the insertion section 411 in the region S1 and the vaginal anterior wall 1410 is suction held onto the insertion section 511 in the region S2, the urethral wall and the vaginal wall are spaced wider apart from each other between the regions S1 and S2. Therefore, by passing the puncture member 3 through such a region, the puncture by the puncture member 3 can be performed relatively safely.

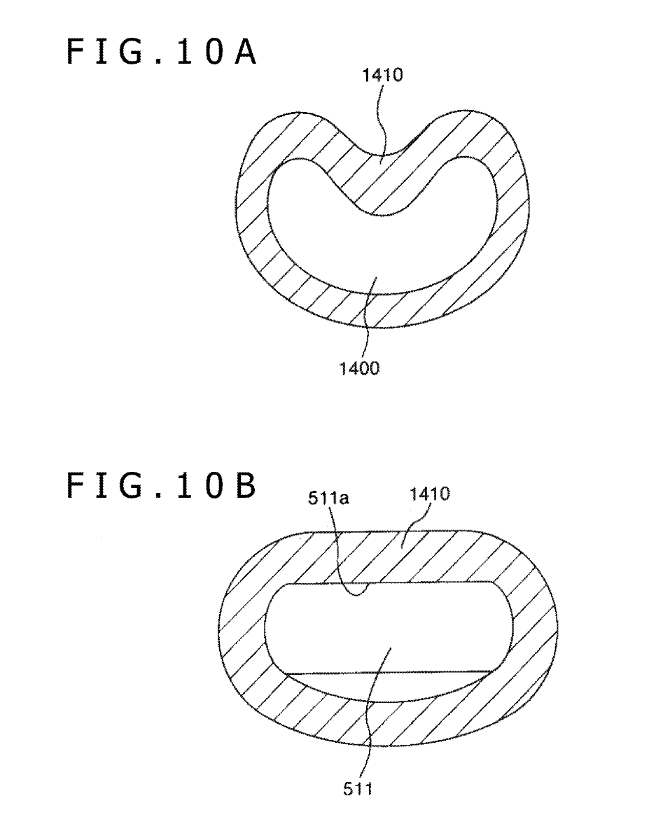

The region S2 stretches over substantially the whole range in the width direction of the upper surface 511a. A width W2 of the region S2 is not particularly limited, and is preferably, for example, about 9 mm to 49 mm, more preferably about 19 mm to 39 mm, which helps enable the vaginal anterior wall 1410 to be suction held onto the insertion section 511 reliably, without being considerably influenced by the shape of the vaginal wall. Especially, for example, a patient may have a vagina 1400 shaped as depicted in FIG. 10A, wherein part of a vaginal anterior wall 1410 droops down into the inside of the vagina. Even in such a case, setting the width W2 as above-mentioned ensures that as depicted in FIG. 10B, not only the drooping-down part but also the parts on both sides of the drooping-down part can be suction held reliably. Therefore, the vaginal anterior wall 1410 can be spaced apart from the urethra 1300 reliably, without being influenced by the shape of the vagina 1400. Particularly, for example, in the present embodiment, the insertion section 511 is flat shaped, so that the vaginal anterior wall 1410 can be suction held in the manner of being spaced farther away from the urethra 1300, and the living body tissue between the urethral wall and the vaginal wall can be widened.

In addition, the insertion section 511 is provided with a marker (puncture position confirmation section) 57 with which the puncture route of the puncture apparatus 1 can be confirmed. In other words, the puncture apparatus 1 can be fixed so as to puncture between the vaginal wall, which is present on the upper side of the position where the marker 57 exists, and the urethral wall. As a result, the operability and safety of the insertion tool 6 can be enhanced. The marker 57 can be provided at least on a lower surface 511b of the insertion section 511. The lower surface 511b is a surface, which is oriented toward the vaginal orifice side and is visible by the operator through the vaginal orifice, in the inserted state. With the marker 57 provided on the lower surface 511b, therefore, the puncture route of the puncture apparatus 1 can be reliably confirmed. In addition, the depth of insertion of the insertion section 511 into the vagina 1400 can also be confirmed. Note that it is sufficient for the marker 57 to be visible externally, and the marker 57 can be configured as a colored part, a rugged part or the like.

In accordance with an exemplary embodiment, the non-insertion section 512 can be in the shape of a thin bar extending substantially in parallel to the urethral-insertion section 41. A separated distance D between the non-insertion section 512 and the urethral-insertion section 41 is not particularly limited, and is preferably, for example, about 5 mm to 40 mm, correspondingly to the separated distance between the urethral orifice and the vaginal orifice in most women.

The length of the non-insertion section 512 (the separated distance between the vaginal orifice and the support section 50) is not specifically restricted, and is preferably, for example, not more than about 100 mm, more preferably in the range of about 20 mm to 50 mm. By this, the non-insertion section 512 can be made to have a suitable length, and its operability is enhanced. If the length of the non-insertion section 512 exceeds the aforesaid upper limit, there may arise, depending on the configuration of the frame 2 or the like, a situation in which the center of gravity of the puncture apparatus 1 is largely spaced from the patient and, accordingly, the stability of the puncture apparatus 1 in the mounted state is lowered.

In accordance with an exemplary embodiment, the support section 50 is provided with a male screw 501. With the male screw 501 fastened into a female screw (not illustrated) provided in the support section 40, the support sections 40 and 50 are fixed to each other.

The material constituting the vaginal-insertion member 5 is not specifically restricted. In this case, there can be used, for example, various metallic materials such as stainless steel, aluminum or aluminum alloys, titanium or titanium alloys, etc. and various resin materials, like in the case of the urethral-insertion member 4.

Note that while the urethral-insertion member 4 and the vaginal-insertion member 5 constituting the insertion tool 6 have been configured to be freely detachable each other in the puncture apparatus 1, this configuration is not restrictive. The urethral-insertion member 4 and the vaginal-insertion member 5 may be so configured that they cannot be attached to or detached from each other.

In addition, while the urethral-insertion section 41 is fixed relative to the support section 40 in the puncture apparatus 1, this configuration is not restrictive. A configuration may be adopted wherein a state where the urethral-insertion section 41 is fixed relative to the support section 40 and a state where the urethral-insertion section 41 is slidable in the axial direction relative to the support section 40 can be selected. Specifically, for example, a configuration may be adopted wherein loosening a screw provided on the support section 40 results in a state where the urethral-insertion section 41 is slidable relative to the support section 40 and wherein fastening the screw results in a state where the urethral-insertion section 41 is fixed relative to the support section 40. According to this configuration, the length of the non-insertion section 412 can be adjusted, so that user-friendly insertion tool 6 is realized. Note that the same applies to the vaginal-insertion section 51.

In addition, while the component members are fixed to the frame 2 so that the inclination angle θ2 is constant in the puncture apparatus 1, this configuration is not restrictive, and the inclination angle θ2 may be variable. Where the inclination angle θ2 is variable, the inclination angle θ2 can be adjusted according to the patient, so that a user-friendly puncture apparatus 1 can be realized.

An implant (implant assembly) 9 to be used with the puncture apparatus 1 will be described below.

As depicted in FIG. 19, the implant (living body tissue-supporting indwelling article) 9 is an embeddable instrument to be placed indwelling between a urethra 1300 and a vagina 1400 for treatment of female urinary incontinence, specifically, an instrument for supporting the urethra 1300. For example, the implant 9 is an instrument which, when the urethra 1300 is going to move toward the vaginal wall side, supports the urethra 1300 so as to restrict such a movement, in the manner of pulling the urethra 1300 in the direction for spacing away from the vaginal wall. As the implant 9, for example, a flexible elongated body can be used.

The implant 9 can include an implant main body (belt-shaped elongated article) 91, and a bag-shaped wrapping material (protection material) 92 for accommodating the implant main body 91. In addition, the implant main body 91 can include a main body section 911, and a ribbon 912 interlocked to one end of the main body section 911.

The main body section 911 is a belt body in a net-like form. Note that the main body section 911 may be composed, for example, of a network-like knitted body knitted by causing linear (filamentous) elements to intersect, specifically, network-like braiding. Examples of the linear element include those which are circular in cross section, and those which are flat shaped in cross section.

The ribbon 912 functions as a guide section which, at the time of inserting the main body section 911 (implant main body 91) into the sheath 30, guides and pulls the main body section 911. Note that a guide wire, a cord, a string or the like may be used in place of the ribbon 912.

The wrapping material 92 is elongated in overall shape, like the implant main body 91, so that it can accommodate the implant main body 91, which can make it possible to effectively prevent contamination of the implant main body 91. In addition, the wrapping material 92 can include a flexible sheet material.

The materials constituting the main body section 911, the ribbon 912, and the wrapping material 92 are not particularly limited. For example, various resin materials which are biocompatible such as polypropylene, polyesters, nylon, etc., fibers and the like can be used as the materials.

Note that the implant 9 is not limited to the above-mentioned network-formed one, so long as the same or equivalent effect can be exhibited.

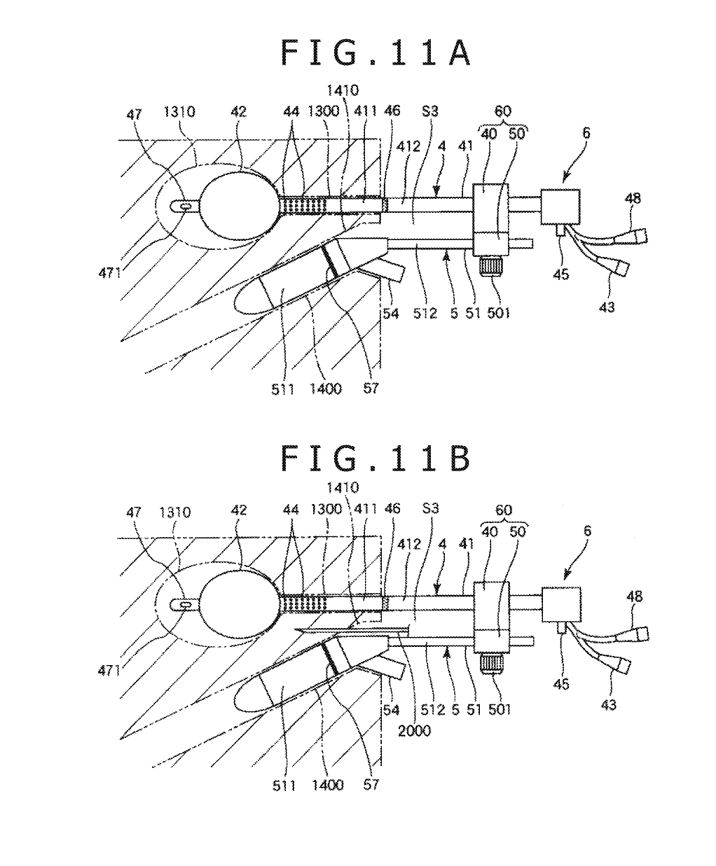

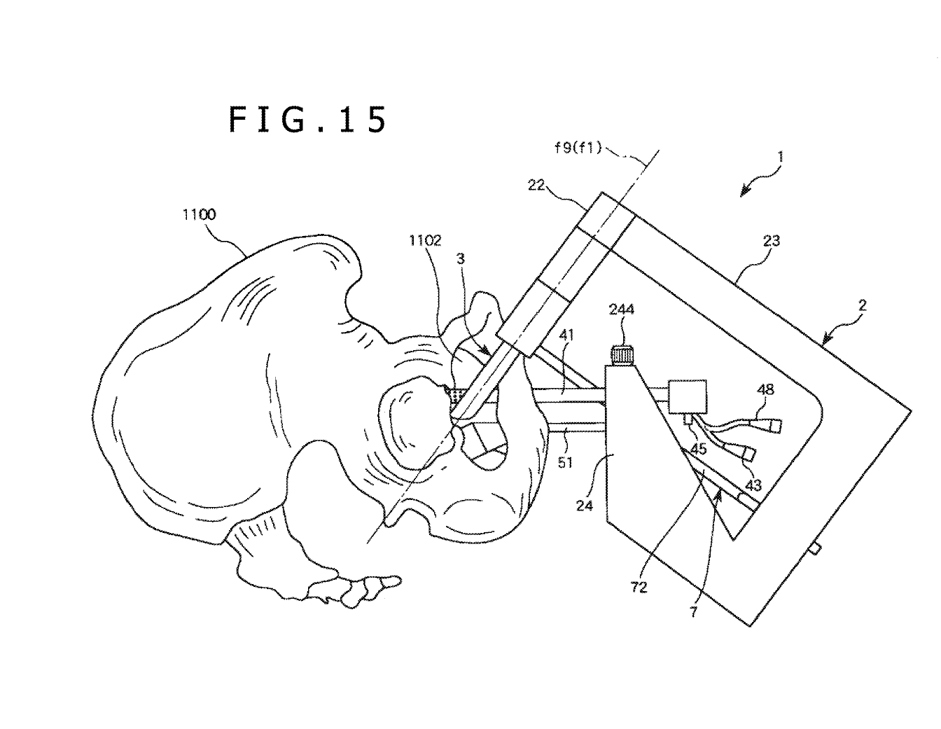

Now, a using method (operating procedure) of the puncture apparatus 1, specifically, a method of placing the implant 9 indwelling by use of the puncture apparatus 1 will be described below, referring to FIGS. 11 to 24. The use of this puncture apparatus 1 can make it possible, at the time of placing the implant 9 indwelling between a urethra 1300 and a vagina 1400, to form in the living body tissue a puncture hole having a circular arc shape for indwelling of the implant 9, prior to placing the implant 9 indwelling. Here, the “circular arc shape” not only means a circular arc shape with a curvature constant along the longitudinal direction of an arc but also can include a bow-like shape with a curvature varying along the longitudinal direction of an arc.

First, a patient is placed in a lithotomy position on an operating table, and the insertion tool 6 is mounted onto the patient, as depicted in FIG. 11A. Specifically, first, the urethral-insertion section 41 of the urethral-insertion member 4 is inserted into the patient's urethra 1300. In this case, the depth of insertion is confirmed with the marker 46, and the balloon 42 is disposed inside the bladder 1310. The urethra 1300 is corrected into a predetermined shape by the urethral-insertion section 41 having the predetermined shape. In the case of the present embodiment, the urethra 1300 is corrected into a rectilinear shape by the urethral-insertion section 41, which is rectilinear in shape.

Next, the balloon 42 is expanded, and urine is drained from within the bladder 1310 via the drain hole 471, as required. In addition, the vaginal-insertion section 51 of the vaginal-insertion member 5 is inserted into the patient's vagina 1400. In this case, the puncture position is confirmed with the marker 57, and insertion into a suitable depth is performed. Then, the support sections 40 and 50 are fixed by operating the male screw 501. By this, the mounting of the insertion tool 6 onto the patient is completed. In this state, the non-insertion sections 412 and 512 are spaced apart from each other, and, further, the support section 60 is spaced apart from a body surface between the urethral orifice and the vaginal orifice, so that the body surface is exposed. In addition, in the case where the insertion section 511 and the vaginal anterior wall 1410 are spaced apart from each other to form a gap (space) therebetween, there is formed a space S3 for permitting a syringe 2000 to puncture living body tissue between the urethra 1300 and the vagina 1400 via the body surface between the urethral orifice and the vaginal orifice.

Subsequently, suction devices are connected to the suction ports 45 and 54, and the suction devices are operated, to suction hold the urethra 1300 onto the urethral-insertion section 41 and suction hold the vaginal anterior wall 1410 onto the vaginal-insertion section 51. For example, when the urethra 1300 is suction held onto the urethral-insertion section 41 properly, the suction holes 44 are closed with the urethral wall, so that the suction via the suction port 45 is stopped or weakened. Similarly, when the vaginal anterior wall 1410 is suction held onto the vaginal-insertion section 51 properly, the suction holes 59 are closed with the vaginal wall, so that the suction via the suction port 54 is stopped or weakened. Therefore, according to the manners of suction via the suction ports 45 and 54 (for example, according to the magnitudes of the sounds generated upon the suction), the operator can check whether or not the urethra 1300 and the vaginal anterior wall 1410 are suction held onto the urethral-insertion section 41 and the vaginal-insertion section 51 properly. Note that the insertion tool 6 may be provided with a checking mechanism for mechanically checking the suction-held state. The checking mechanism is not specifically restricted, so long as the suction-held state can be checked by use of the mechanism. For example, there may be adopted a configuration including a flow rate measuring section (negative pressure meter) for measuring the flow rate through the suction port 54, and a determining section for determining whether or not the suction holding is performed properly, on the basis of the measurement results supplied from the flow rate measuring section.

Next, liquid dissection is conducted. Specifically, as depicted in FIG. 11B, a puncture needle of a syringe 2000 is made to puncture the vaginal anterior wall 1410 through the space (space S3) between the insertion section 511 and the vaginal anterior wall 1410, and a liquid such as physiological saline solution or local anesthetic is injected into the living body tissue in a region between the urethra 1300 and the vagina 1400 (a region between the region S1 and the region S2). As a result, the living body tissue between the regions S1 and S2 is expanded, the urethra 1300 is pressed against the urethral-insertion section 41, and the vaginal anterior wall 1410 is pressed against the vaginal-insertion section 51.

Here, it can be preferable to continue the suction via the suction holes 44 and 59 even during the liquid dissection. When the urethra 1300 is pressed against the urethral-insertion section 41 by the liquid dissection, the urethra 1300 is suction held onto the urethral-insertion section 41 securely, so that the suction through the suction port 45 is stopped or weakened. Similarly, when the vaginal anterior wall 1410 is pressed against the vaginal-insertion section 51, the vaginal anterior wall 1410 is suction held onto the vaginal-insertion section 51 securely, so that the suction through the suction port 45 is stopped or weakened. Therefore, according to the manners of suction via the suction ports 45 and 54, the operator can check whether or not the liquid dissection is performed properly.

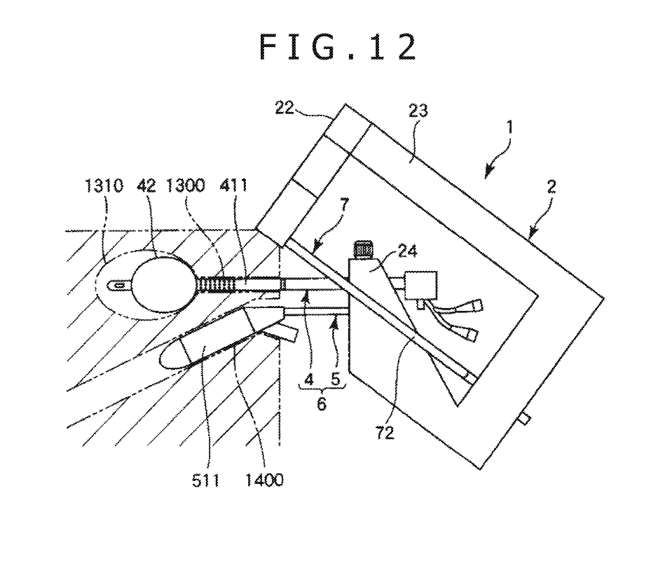

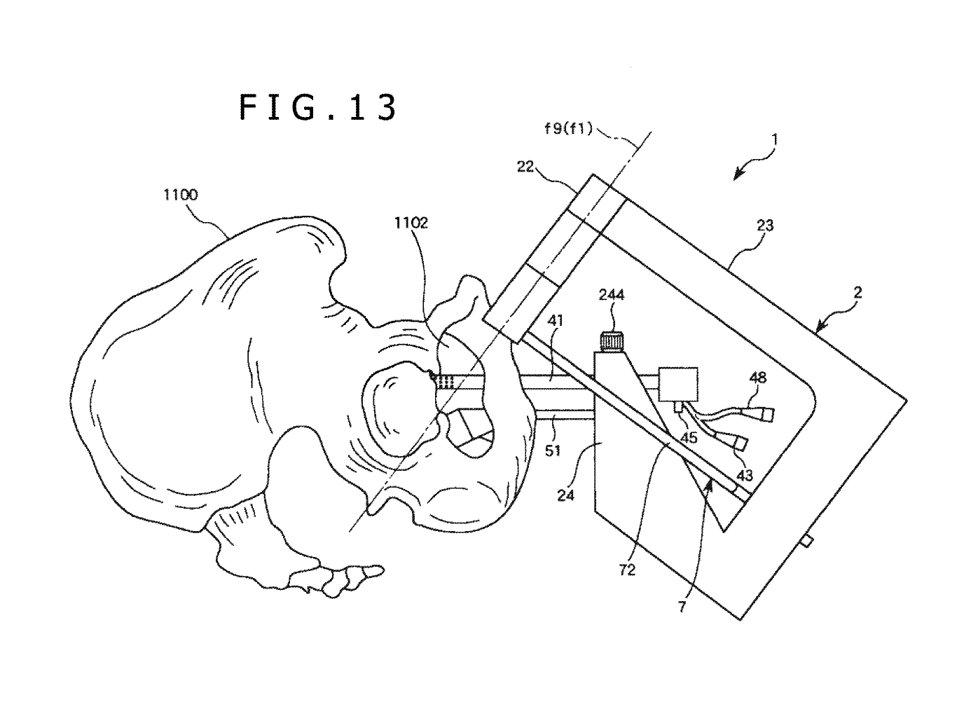

After the liquid dissection is performed and the urethra 1300 and the vaginal anterior wall 1410 are sufficiently spaced apart, the frame 2 is fixed to the insertion tool 6, as depicted in FIG. 12. This results in a state in which the puncture apparatus 1 is mounted onto the patient. In this state, the positional relation between the pelvis 1100 and the puncture apparatus 1 is as depicted in FIG. 13.

Note that the sheath 30 supported by the frame 2 in this instance is in a state where the extension tube 37 is not yet connected to the main body 31 and where the needle body 35 instead of the extension tube 37 is in connection with the main body 31.

Subsequently, for example, while gripping the interlock section 23 of the frame 2 by one hand, the interlock section 72 of the operating member 7 is grasped by the other hand, and, as depicted in FIG. 14, the operating member 7 is rotated clockwise, which causes the needle body 35 of the puncture member 3 to puncture a body surface H at a part (first part) in an inguinal region on the right side of the patient or near the inguinal region, thereby entering the body with the main body 31, to sequentially pass an obturator foramen 1101 on one side, between the urethra 1300 and the vagina 1400, and an obturator foramen 1102 on the other side, then to exit the body via the body surface H at a part (second part) in an inguinal region on the left side or near this inguinal region (see FIG. 14).

In addition, in this state, as depicted in FIG. 14, the body surface H is pressed down by each lower end portion of the guide section 22 of the frame 2, whereby a hollowed portion H1 hollowed in the surroundings of the distal side of the main body 31 is formed on the distal side of the main body 31, and a hollowed portion H2 hollowed in the surroundings of the proximal side of the main body 31 is formed on the proximal side of the main body 31. Note that the hollowed portion H2 can be said to be formed also by pressing-down by the flange portion 314 of the main body 31. The needle body 35 is exposed from the hollowed portion H1, and a portion near the proximal-side opening 332 of the main body 31 is exposed from the hollowed portion H2.

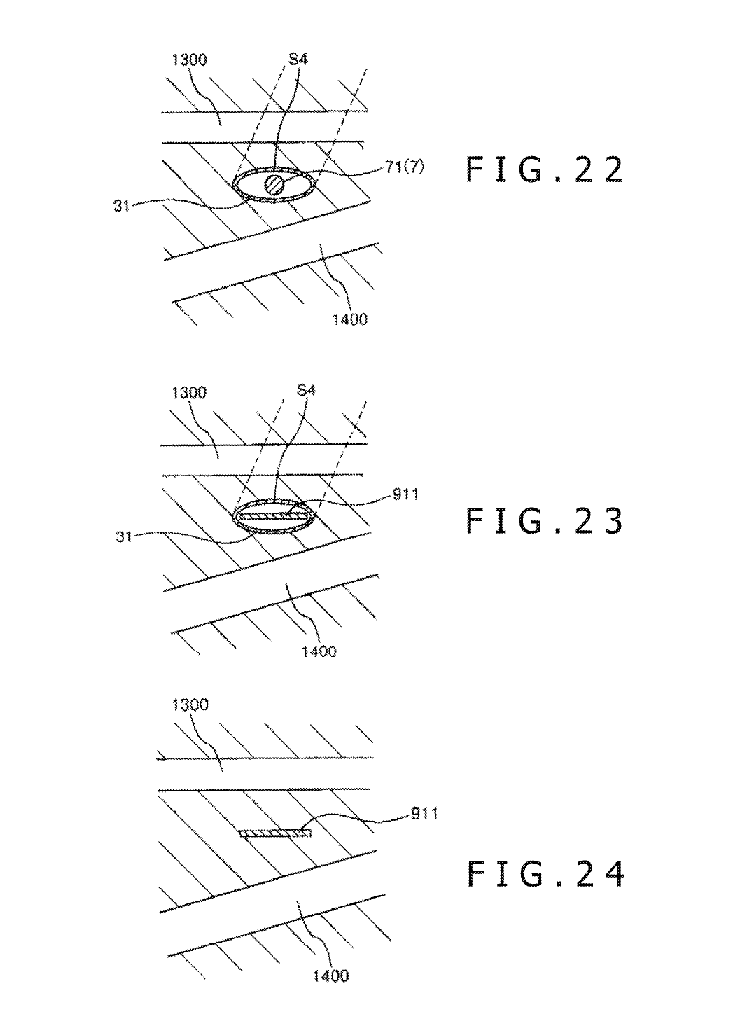

In addition, between the urethra 1300 and the vagina 1400, the central portion S4 of the main body 31 is in a state as depicted in FIG. 22.

Next, a fingertip is inserted into the guide groove 221 of the frame 2 so as to catch on the needle body 35, and, as illustrated in FIG. 16, the needle body 35 is directly detached from the main body 31. Thereafter, the operating member 7 is rotated counterclockwise in FIG. 16. In this instance, the puncture member 3 is also going to rotate counterclockwise together with the operating member 7, but further rotation (movement) of the puncture member 3 is prevented by an engaging mechanism (not depicted). Therefore, the insertion section 71 is drawn out of the puncture member 3 and the living body while there is maintained the state where the distal-side opening 321 of the main body 31 is exposed from the hollowed portion H1 and where the proximal-side opening 332 is exposed from the hollowed portion H2.

Subsequently, the extension tube 37 is inserted into the guide groove 221 of the frame 2, and while keeping the extension tube 37 along the guide groove 221, the extension tube 37 is connected to an end portion located on the forward side with respect to the insertion direction of the main body 31, namely, connected to the distal-side opening 321 side of the main body 31, as depicted in FIG. 17. As a result, the sheath 30 is in the extension tube connected state.

Thereafter, the puncture apparatus 1 (the members other than the sheath 30) is dismounted from the patient. This results in a condition where only the sheath 30 in the extension tube connected state is disposed inside the living body, as depicted in FIG. 18.

In addition, with the puncture apparatus 1 dismounted, the condition where the body surface H is pressed down by each lower end portion of the guide section 22 of the frame 2 is canceled. As a result, the hollowed portions H1 and H2 disappear due to a restoring force of the body surface H, as depicted in FIG. 18. In this instance, the sheath 30 has its flange portion 314 pulled in the proximal direction by the body surface H restored into an original state. As a result, a proximal portion of the extension tube 37 is embedded into the living body together with a distal portion of the main body 31. Since the overall length of the extension tube 37 is longer than the amount of this embedding, a distal-side opening 377 of the extension tube 37 is still in the state of being exposed from the body surface H. In this way, the overall length of the sheath 30 is preliminarily extended prior to placing the implant 9 indwelling, whereby it can be relatively ensured that both end openings of the sheath 30 can be left exposed from the body surface H reliably even after the body surface H is restored into its original state.

Next, the position of the sheath 30 inserted between the urethra 1300 and the vagina 1400 is adjusted, as required. Specifically, the central portion S4 of the main body 31 is positioned between the urethra 1300 and the vagina 1400. In this state, the central portion S4 is so disposed that its width direction (the major axis J32 direction) is substantially parallel to the urethra 1300 (see FIG. 22). In other words, the urethra 1300 corrected in shape due to the insertion of the urethral-insertion member 4 therein and the width direction of the central portion S4 are located substantially in parallel with each other.

Subsequently, as illustrated in FIG. 19, while taking the implant main body 91 out of the wrapping material 92, the implant main body 91 is temporarily inserted into the sheath 30 gradually from the extension tube 37 side. Then, the insertion is further continued, resulting in a state where the ribbon 912 protrudes from the proximal-side opening 332 of the sheath 30 and where a proximal portion of the main body section 911 protrudes from the distal-side opening 377 of the extension tube 37, as depicted in FIG. 20.

Note that in the implant 9, the implant main body 91 can be kept accommodated inside the wrapping material 92 until immediately before disposed inside the sheath 30. By this, contamination of the implant main body 91 can be prevented. In addition, as has been described above, since the main body 31 is flat shaped, the posture of the main body section 911 follows this flat shape. Specifically, as depicted in FIG. 23, the main body section 911 is disposed inside the main body 31 of the sheath 30 in such a manner that its width direction coincides with the width direction of the main body 31. As for the relation with the urethra 1300, the implant main body 91 is disposed in parallel to the urethra 1300, which has been corrected in shape.

Next, the suction holding of the urethra 1300 by the urethral-insertion section 41 and the suction holding of the vaginal anterior wall 1410 by the vaginal-insertion section 51 are stopped. As a result, the positions and shapes of the urethra 1300 and the vagina 1400 are returned into the original natural states.

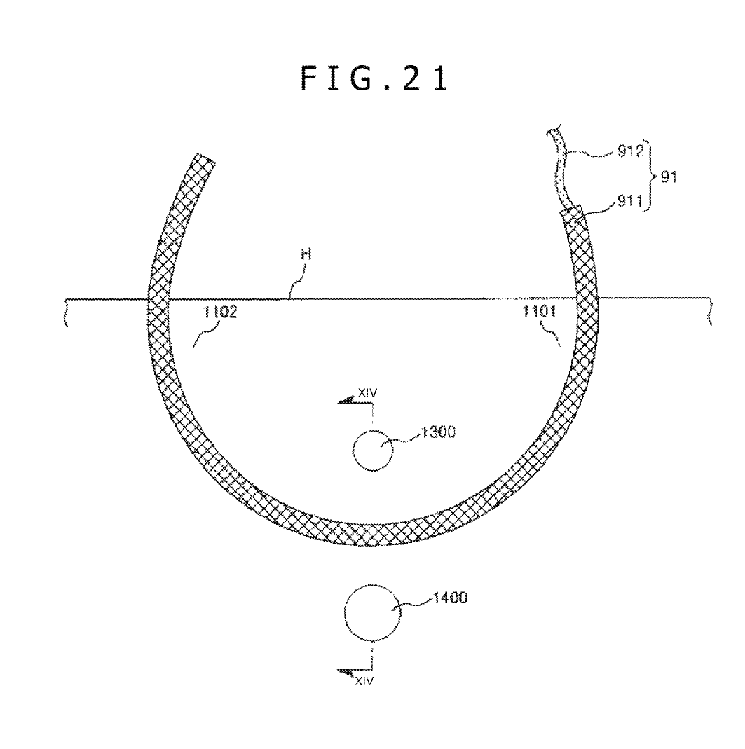

Subsequently, only the sheath 30 is drawn out of the living body starting from the proximal side, while leaving the implant main body 91 inside the living body, as depicted in FIG. 21. As the sheath 30 is gradually removed out of the living body, the surrounding tissue having been pushed open by the sheath 30 returns into its original position, and the tissue comes into contact with the implant main body 91 gradually from a distal portion toward a proximal portion of the implant main body 91. By such an operation, the implant main body 91 can be left indwelling as it is, without any unnecessary tension exerted thereon. As a result, it is unnecessary to adjust a tension on the implant main body 91. The above operations result in a state in which the implant main body 91 is embedded in the living body, as depicted in FIG. 24.

In the state where the implant main body 91 is embedded inside the living body, the main body section 911 is disposed substantially in parallel to the urethra 1300, in a region between the urethra 1300 and the vagina 1400. Therefore, the urethra 1300 can be supported in a wider area by the implant main body 91.

Next, the urethral-insertion member 4 is drawn out of the urethra 1300, and the vaginal-insertion member 5 is drawn out of the vagina 1400. After the urethral-insertion member 4 is drawn out, the urethra 1300 returns into its shape in the natural state. Since the main body section 911 is embedded in the tissue, however, a state in which the urethra 1300 in the natural state and the main body section 911 are parallel can be maintained.

Thereafter, unnecessary portions of the implant main body 91 are cut away, to finish the procedure.

As has been described above, according to the puncture apparatus 1, at the time of temporarily inserting and passing the implant main body 91 into and through the sheath 30, the sheath 30 can be set into the extension tube connected state, so that both ends of the sheath 30 can be reliably exposed from the body surface H irrespectively of the patient's body type. Then, the implant main body 91 can be easily and assuredly inserted via the exposed end portion of the sheath 30. In addition, placement of the implant 9 indwelling can be dealt with by only low-invasive procedures such as puncture by the puncture member 3, and without need for high-invasive procedures such as incision. Therefore, the burden on the patient can be relatively light, and the safety of the patient can be relatively high. In addition, since the implant main body 91 can be embedded in parallel to the urethra 1300, the urethra 1300 can be supported in a wider area by the implant main body 91, and damage to the vagina 1400 or the urethra 1300 by the implant main body 91 can be prevented. In addition, the living body can be punctured by the puncture member 3 while avoiding the urethra 1300 and the vagina 1400, so that puncture of the urethra 1300 or the vagina 1400 with the puncture member 3 can be prevented from occurring, and safety is therefore relatively ensured. Further, unlike in the case of conventional incision of the vagina, a situation can be obviated in which the implant 9 would be exposed to the inside of the vagina via a wound caused by the incision, or a situation in which complications would be generated such as infection from the wound. Thus, very high safety can be ensured, and the implant 9 can be reliably embedded.

FIG. 26 is a cross-sectional view of an extension tube possessed by a medical tube according to a second embodiment of the present disclosure.

Referring to this figure, the second embodiment of the medical tube, a medical device set, and a method of placing an implant indwelling according to the present disclosure will be described below. In the following, description will be made primarily of differences from the aforementioned embodiment, and descriptions of the same items as above will be omitted.

The second embodiment is the same as the first embodiment above, except for a difference in the cross-sectional shape of extension tube.

Where the cross-sectional shape of a main body 31 of a sheath 30 is a circle, it can be preferable that the cross-sectional shape of the extension tube is also a circle, as depicted in FIG. 26, which can make it relatively easy to perform an operation to establish the extension tube connected state.

FIG. 27 is a cross-sectional view of an extension tube possessed by a medical tube according to a third embodiment of the present disclosure.

Referring to this figure, the third embodiment of the medical tube, a medical device set, and a method of placing an implant indwelling according to the present disclosure will be described below. In the following, description will be made primarily of differences from the aforementioned embodiments, and descriptions of the same items as above will be omitted.

The third embodiment is the same as the first embodiment above, except for a difference in the cross-sectional shape of extension tube.

The cross-sectional shape of a main body 31 of a sheath 30 is a flat shape. Where the flat shape is a rhombus with corners somewhat rounded, it can be preferable that the cross-sectional shape of the extension tube is also a rhombus with corners somewhat rounded, as depicted in FIG. 27.

FIG. 28 is a cross-sectional view of an extension tube possessed by a medical tube according to a fourth embodiment of the present disclosure.

Referring to this figure, the fourth embodiment of the medical tube, a medical device set, and a method of placing an implant indwelling according to the present disclosure will be described below. In the following, description will be made primarily of differences from the aforementioned embodiments, and descriptions of the same items as above will be omitted.

The fourth embodiment is the same as the first embodiment above, except for a difference in the cross-sectional shape of extension tube.

Where the cross-sectional shape of a main body 31 of a sheath 30 is a circle, it can be preferable that the cross-sectional shape of the extension tube is also a circle, as depicted in FIG. 28. In addition, it is more preferable that there is formed a cutout 373 where part of the circle is cut out.

In the case of connecting the extension tube 37 configured as above to the main body 31, the connecting operation may be conducted by bringing the extension tube 37 closer to the main body 31 from the distal side of the main body 31. A method may be adopted, however, in which while bringing the extension tube 37 closer to the main body 31 from a lateral side of the main body 31, the cutout 373 of the extension tube 37 is temporarily opened wide, and the connecting operation is performed.

For example, in the case where other medical device than the puncture apparatus 1 is present in the vicinity of the patient and it is difficult to connect the extension tube 37 to the main body 31 from the distal side of the main body 31, the connecting operation can be easily carried out by connecting the extension tube 37 to the main body 31 from a lateral side of the main body 31.

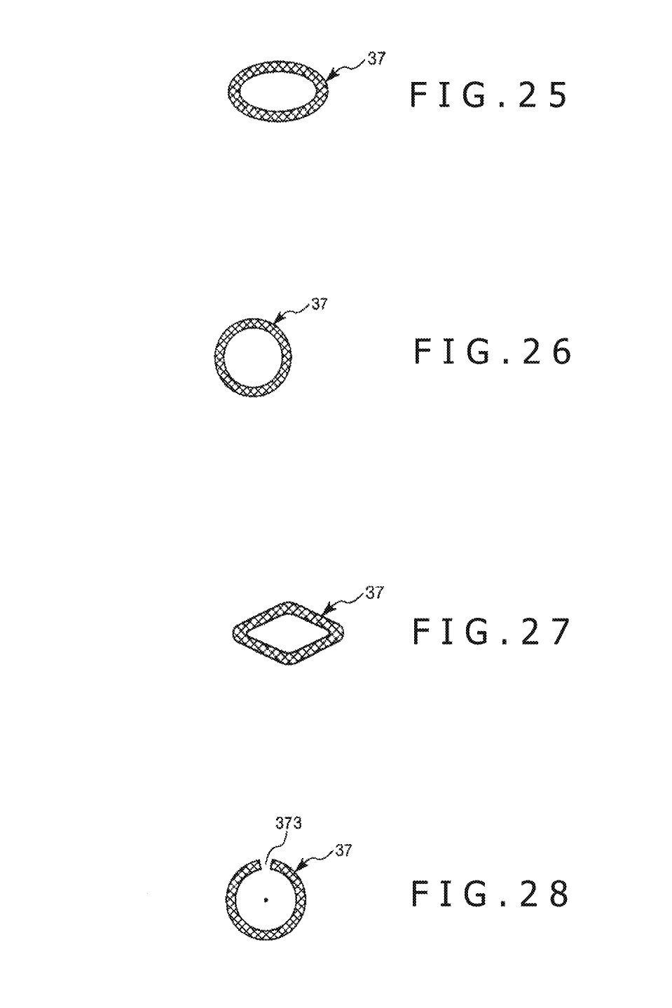

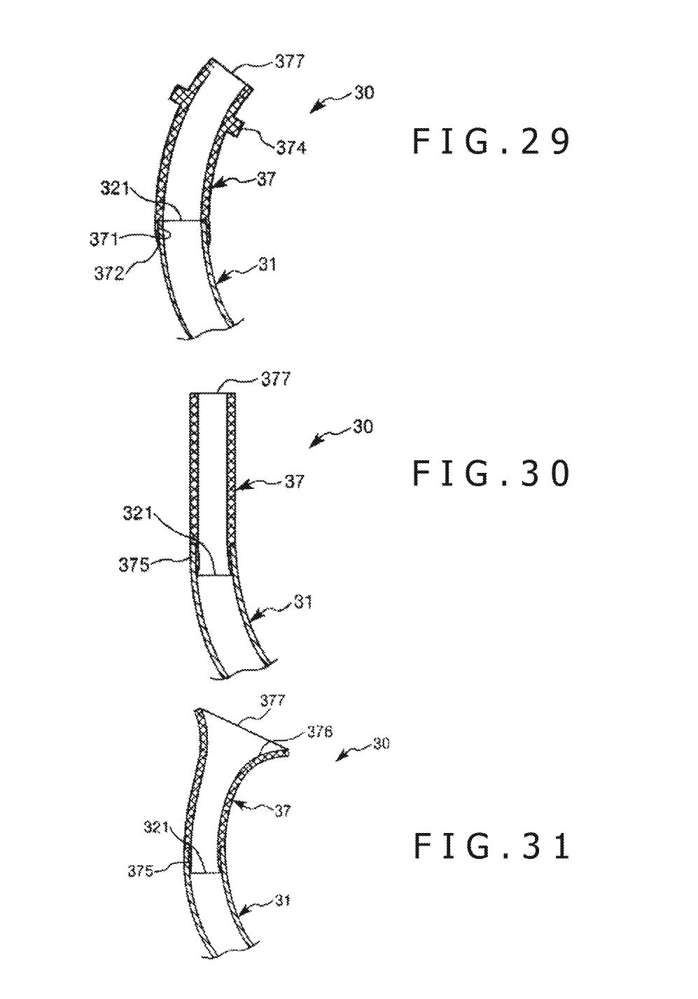

FIG. 29 is a longitudinal sectional view of an extension tube possessed by a medical tube according to a fifth embodiment of the present disclosure.

Referring to this figure, the fifth embodiment of the medical tube, a medical device set, and a method of placing an implant indwelling according to the present disclosure will be described below. In the following, description will be made primarily of differences from the aforementioned embodiments, and descriptions of the same items as above will be omitted.

The fifth embodiment is the same as the first embodiment, except for a difference in the configuration of extension tube.

As illustrated in FIG. 29, in the present embodiment, the extension tube 37 is provided at a longitudinally intermediate portion thereof with a flange portion 374 composed of an enlarged diameter portion enlarged in outside diameter. With the flange portion 374, a body surface H may be engaged upon disappearance of a hollowed portion H1 of the body surface H. This engagement can enable a distal-side opening 377 of the extension tube 37 to be assuredly exposed from the body surface H. Consequently, an implant 9 can be inserted into a sheath 30 through the distal-side opening 377.

Note that the flange portion 374 may be formed to be integral with the extension tube 37, or may be configured as a separate body from the extension tube 37 and fixed to the extension tube 37.

FIG. 30 is a longitudinal sectional view of an extension tube possessed by a medical tube according to a sixth embodiment of the present disclosure.

Referring to this figure, the sixth embodiment of the medical tube, a medical device set, and a method of placing an implant indwelling according to the present disclosure will be described below. In the following, description will be made primarily of differences from the aforementioned embodiments, and descriptions of the same items as above will be omitted.

The sixth embodiment is the same as the first embodiment above, except for a difference in the external shape of extension tube.

As depicted in FIG. 30, in the present embodiment, the extension tube 37 is a member which is straight in shape. For example, some operators of a puncture apparatus 1, or some medical staff may feel that a straight extension tube 37 is more user-friendly. In such a case, the extension tube 37 according to the present embodiment can be effective. In addition, since the extension tube 37 can be inserted vertically from directly above the patient, there is also a merit that the insertion is relatively easy to carry out.

In addition, the extension tube 37 is provided at a proximal outer circumferential portion thereof with a reduced diameter portion 375 reduced in outside diameter. At the time of connecting the extension tube 37 to a main body 31, a distal portion of the main body 31 can be externally fitted onto the reduced diameter portion 375. As a result, the extension tube connected state is established.

FIG. 31 is a longitudinal sectional view of an extension tube possessed by a medical tube according to a seventh embodiment of the present disclosure.

Referring to this figure, the seventh embodiment of the medical tube, a medical device set, and a method of placing an implant indwelling according to the present disclosure will be described below. In the following, description will be made primarily of differences from the aforementioned embodiments, and descriptions of the same items as above will be omitted.

The seventh embodiment is the same as the sixth embodiment above, except for a difference in the configuration of extension tube.

As depicted in FIG. 31, in the present embodiment, the extension tube 37 is curved in a circular arc shape. Of the extension tube 37, an end portion on the opposite side from the side of connection with a main body 31, namely, for example, a distal portion of the extension tube 37 can be enlarged in diameter in a trumpet-shaped form, to constitute an easy-introduction portion 376 which facilitate insertion of an implant main body 91.

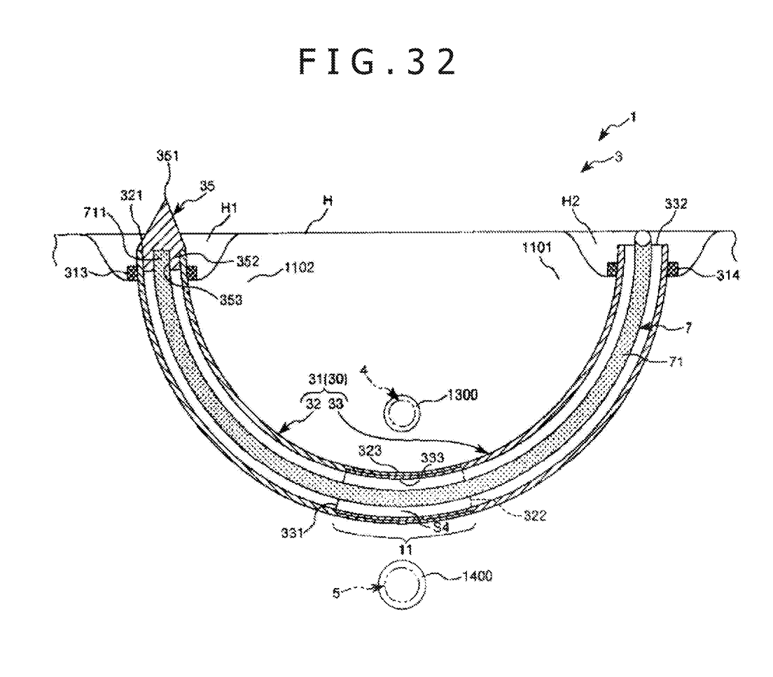

FIGS. 32, 33, and 34 are views (as viewed from the side of the patient's legs) for explaining an operating procedure of a puncture apparatus where a medical tube according to an eighth embodiment of the present disclosure is applied to the puncture apparatus.

Referring to these figures, the eighth embodiment of the medical tube, a medical device set, and a method of placing an implant indwelling according to the present disclosure will be described below. In the following, description will be made primarily of differences from the aforementioned embodiments, and descriptions of the same items as above will be omitted.

The eighth embodiment is the same as the first embodiment above, except for differences in the configuration of sheath.

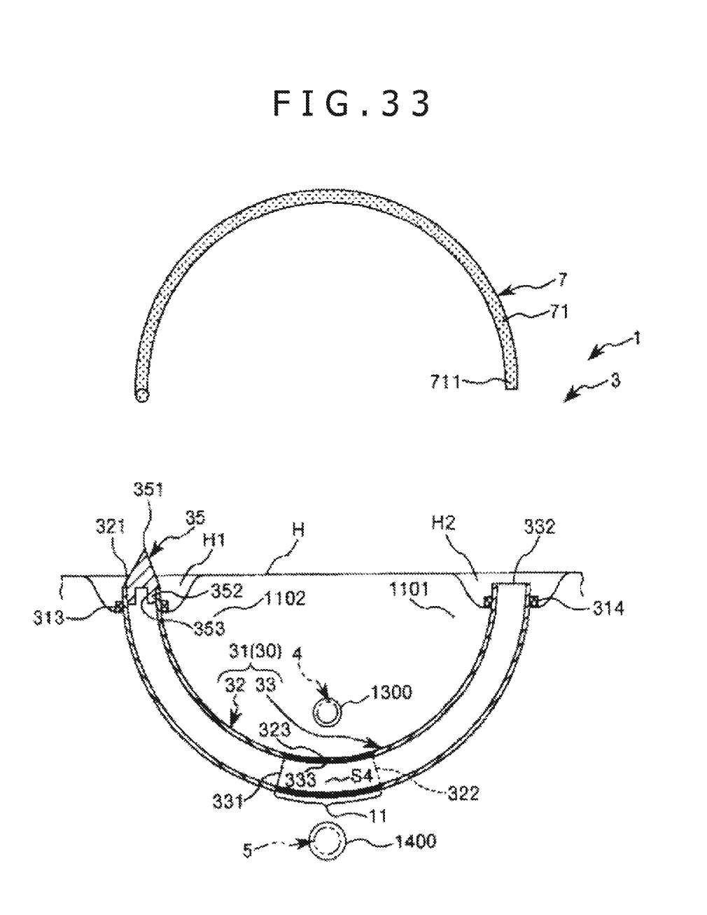

As illustrated in FIGS. 32 and 33, in the present embodiment, a main body 31 of a sheath 30 can include two separable pieces so interlocked to each other that they are separable at an intermediate portion. Specifically, the main body 31 is configured by interlocking a distal separable piece 32 and a proximal separable piece 33 to each other along a longitudinal direction of the main body 31. The distal separable piece 32 and the proximal separable piece 33 are substantially the same in length.

The distal separable piece 32 is a tube shaped member, and has a distal-side opening 321 and a proximal-side opening 322. Similarly, the proximal separable piece 33 is a tube shaped member, and has a distal-side opening 331 and a proximal-side opening 332.

A distal portion 333 of the proximal separable piece 33 is inserted in a proximal portion 323 of the distal separable piece 32, whereby the distal separable piece 32 and the proximal separable piece 33 can be moved relative to each other along a longitudinal direction of the sheath 30. After a puncture member 3 punctures a living body, the overall length of the sheath 30 can be extended by this movement. Thus, in the present embodiment, the part at which the distal portion 333 of the proximal separable piece 33 is inserted in the proximal portion 323 of the distal separable piece 32 and which is supported in a movable manner can be said to be an “extension section 11” for extending the overall length of the sheath 30. The extension section 11 is preferably located at a central portion S4.

Note that the distal separable piece 32 and the proximal separable piece 33 communicate with each other, irrespectively of the degree of extension, or at least after an extending operation by the extension section 11. In addition, the distal separable piece 32 and the proximal separable piece 33 may be connected together by inserting the distal separable piece 32 into the proximal separable piece 33, conversely to the present embodiment.

In accordance with an exemplary embodiment, the thickness of a tube wall of the proximal portion 323 of the distal separable piece 32 can be ½ times the thickness of a tube wall of a portion on the distal side of the proximal portion 323. In addition, the thickness of a tube wall of the distal portion 333 of the proximal separable piece 33 can be ½ times the thickness of a tube wall of a portion on the proximal side of the distal portion 333. By such a thickness magnitude relation, the thickness of the tube wall at the extension section 11 can be prevented from becoming excessively large. In other words, the thickness of the tube wall at the extension section 11 can be made to be the same as the thickness of the tube wall on the forward side and the backward side of the extension section 11. As a result, generation of a step at the boundary between the distal separable piece 32 and the proximal separable piece 33 can be prevented. Therefore, an operation of inserting and passing the sheath 30 into and through a living body can be performed relatively smoothly. In addition, an operation of inserting and passing an implant 9 into and through the sheath 30 can be carried out relatively smoothly.

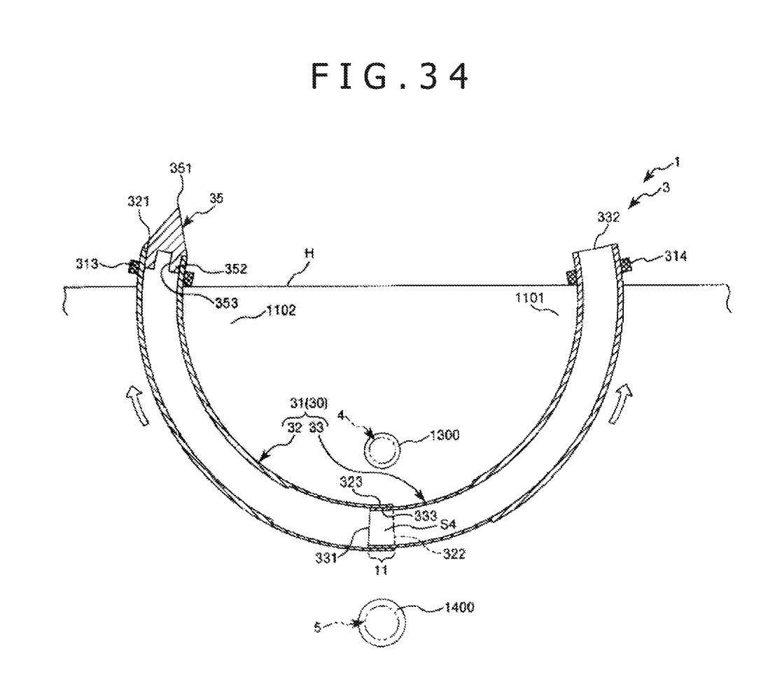

In addition, at the extension section 11, the distal separable piece 32 and the proximal separable piece 33 can engage each other, in a state where the sheath 30 is extended maximally as depicted in FIG. 34. By the engagement, further movement of each separable piece is prevented, so that unintended slip-off of the proximal separable piece 33 from the distal separable piece 32 can be prevented from occurring.

While the number of the separable pieces arranged to configure the main body 31 of the sheath 30 is two in the present embodiment, the number is not particularly limited, and may, for example, be three or more.

In accordance with an exemplary embodiment, to a distal portion of the distal separable piece 32, a flange portion 313 as an attachment member attaching a body surface H is mounted in a freely detachable manner. The flange portion 313 is a portion, which has an outside diameter greater than the outside diameter of the distal separable piece 32.

In a state before the puncture member 3 punctures a living body and the extension section 11 operates, the flange portion 313 is not yet mounted to the distal separable piece 32 but is accommodated and disposed inside the guide groove 221 of the frame 2. Then, when puncture of the living body by the puncture member 3 is completed as depicted in FIG. 32, the distal separable piece 32 passes through the flange portion 313 to fit inside the flange portion 313. As a result, the flange portion 313 is mounted to the distal separable piece 32.

As the flange portion 314 is mounted to a proximal portion of the proximal separable piece 33, the flange portion 313 mounted to the distal separable piece 32 can attach the body surface H and press the body surface H. By this pressing, the body surface H is pressed down, and a hollowed portion H1 is thereby formed. Then, when the hollowed portions H1 and H2 disappear due to a restoring force of the body surface H as depicted in FIGS. 33 and 34, the distal separable piece 32 has the flange portion 313 pulled in the distal direction by the body surface H restored in shape; also, the proximal separable piece 33 has the flange portion 314 pulled in the proximal direction by the body surface H restored in shape. As a result, the extension section 11 operates, resulting in a state where the overall length of the sheath 30 is extended sufficiently and assuredly.

A method of using the puncture apparatus 1 according to the eighth embodiment will be described below with reference to FIGS. 32 to 34.

The puncture apparatus 1 is set into the state depicted in FIG. 12, in the same manner as in the method of using the puncture apparatus 1 in the first embodiment. Thereafter, as depicted in FIG. 32, the operating member 7 is rotated clockwise. By this operation, the needle body 35 of the puncture member 3 is caused to puncture the body surface H at an inguinal region on the right side of the patient or at a neighboring part, to enter the body together with the main body 31 in the state of being not yet extended in overall length, then to pass an obturator foramen 1101 on the one side, between the urethra 1300 and the vagina 1400, and an obturator foramen 1102 on the other side, and to exit the body via the body surface H at an inguinal region on the left side or at a neighboring part (second part). In this instance, the flange portion 313 is mounted to the distal separable piece 32 of the main body 31.

In addition, in this state, as depicted in FIG. 32, a lower end portion on one side of the guide section 22 of the frame 2 presses down the body surface H together with the flange portion 313, to form a hollowed portion H1 in a surrounding area. Similarly, a lower end portion on the other side of the guide section 22 presses down the body surface H together with the flange portion 314, to form a hollowed portion H2 in a surrounding area.

Next, as illustrated in FIG. 33, the operating member 7 is rotated counterclockwise in the figure, and is thereby drawn out of the puncture member 3. In this instance, although the puncture member 3 is going to rotate counterclockwise together with the operating member 7, the engagement of the flange portion 313 with the body surface H help prevent further rotation of the puncture member 3.

Thereafter, the puncture apparatus 1 (the members other than the puncture member 3) is dismounted from the patient. As a result, as depicted in FIG. 34, the pressed-down state of the body surface H is canceled, and the hollowed portions H1 and H2 disappear due to the restoring force of the body surface H. In addition, owing to the restoring force of the body surface H, the distal separable piece 32 is pulled in the distal direction through the function of the flange portion 313, and the proximal separable piece 33 is pulled in the proximal direction through the function of the flange portion 314. Consequently, the extension section 11 operates, resulting in a state where the overall length of the sheath 30 is extended. In this state, the distal-side opening 321 of the distal separable piece 32 and the proximal-side opening 332 of the proximal separable piece 33 are located above the body surface H.

Subsequently, the needle body 35 is drawn out of the distal separable piece 32. Thereafter, an operation of placing the implant 9 indwelling as illustrated in FIG. 19 and the latter figures is conducted, in the same manner as in the method of using the puncture apparatus 1 in the first embodiment.

Thus, according to the puncture apparatus 1, at the time of placing the implant 9 indwelling in a living body, the sheath 30 can be set into the extended state prior to the placement of the implant 9. The sheath 30 in the extended state has both its ends exposed from the body surface H in a reliable manner. Then, after the implant 9 is temporarily inserted via the exposed part, the operation of placing the implant 9 indwelling in the living body can be carried out.

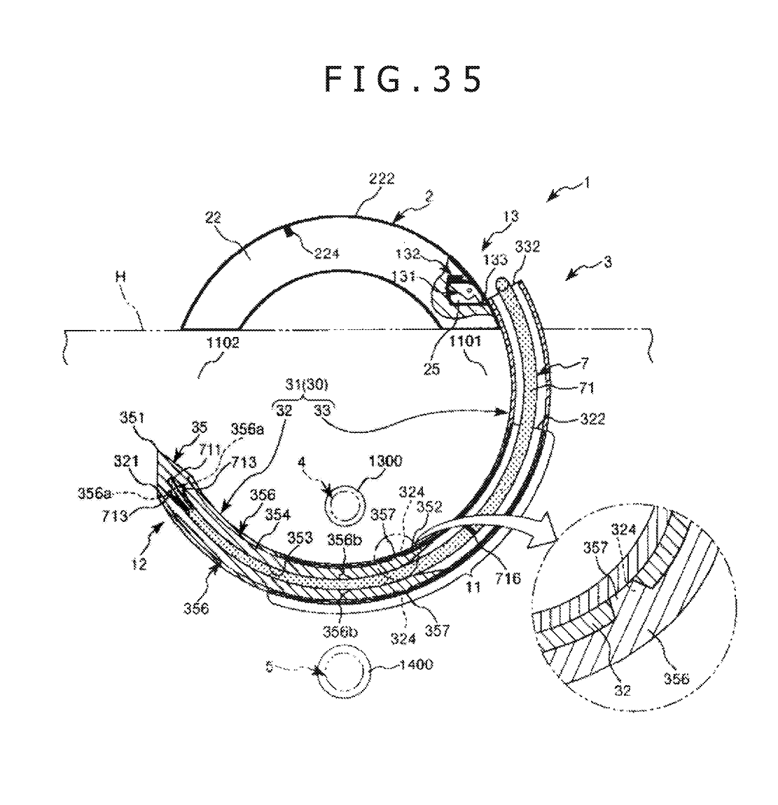

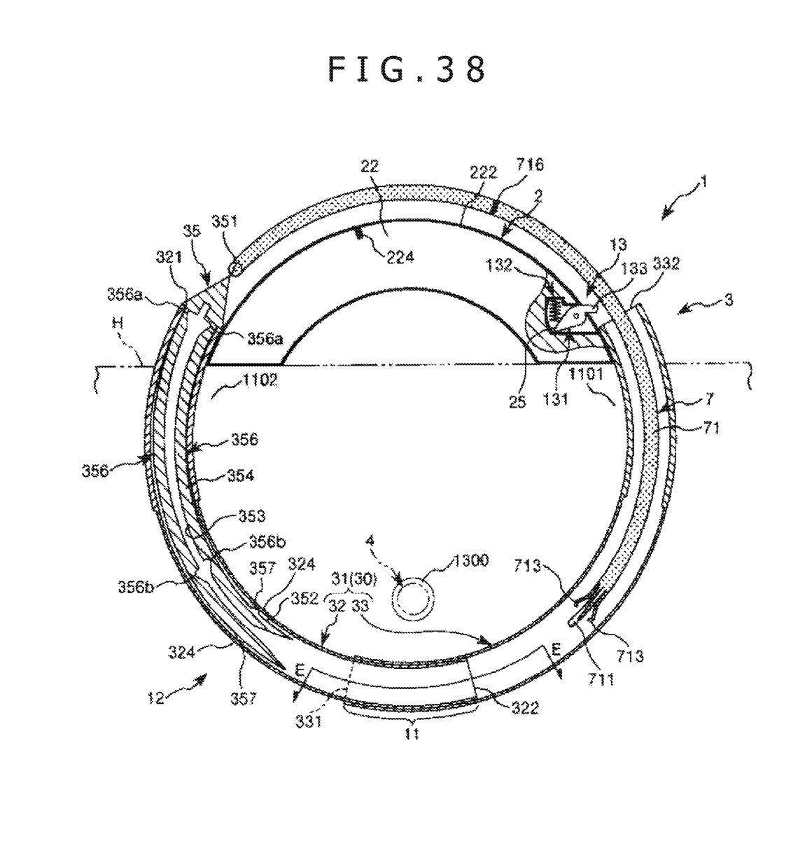

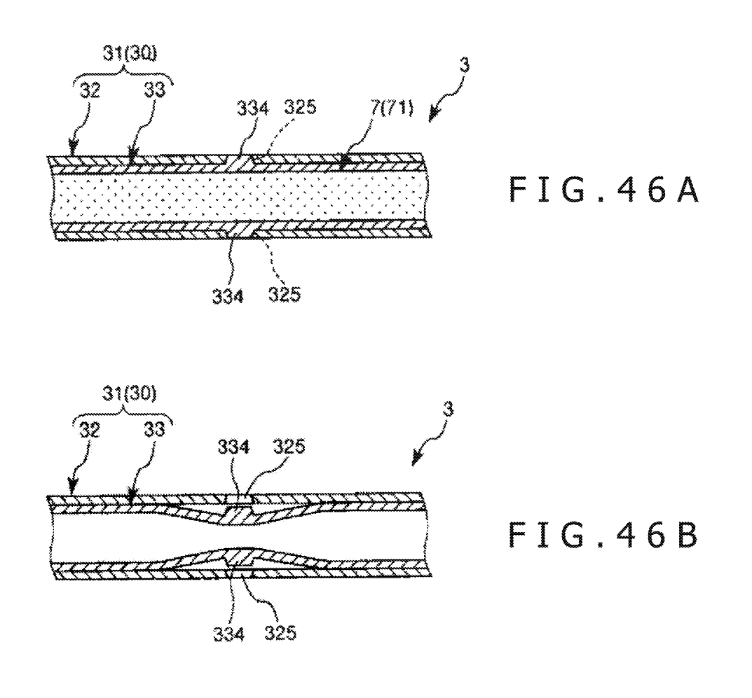

FIGS. 35 to 45 are views (as viewed from the side of the patient's legs) for explaining an operating procedure of a puncture apparatus in the case where a medical tube according to a ninth embodiment of the present disclosure is applied to the puncture apparatus. FIG. 46 is a sectional view taken along line XXXXVI-XXXXVI of FIG. 38.

Referring to these figures, the ninth embodiment of the medical tube, a medical device set, and a method of placing an implant indwelling according to the present disclosure will be described below. In the following, description will be made primarily of differences from the aforementioned embodiments, and descriptions of the same items as above will be omitted.

The ninth embodiment is the same as the second embodiment above, except for differences in the configuration of puncture member.

As illustrated in FIGS. 35 to 40, 44, and 45, in the present embodiment, the needle body 35 of the puncture member 3 has an elongated intermediate portion 354 between the needle tip 351 and the proximal section 352. The intermediate portion 354, together with the proximal section 352, constitutes a pair of elastic pieces 356, which are oppositely disposed and are biased toward each other by their own elastic forces. The elastic pieces are each curved in a circular arc shape. In addition, the part between the elastic pieces 356 constitutes the engaging section 353 for engagement with the insertion section 71. The cross-sectional shape of the elastic pieces 356 is not particularly restricted, and may be, for example, a polygon, a circle, or an ellipse.

The puncture member 3 further can include a locking section 12 capable of assuming a locked state (see FIGS. 35 to 37) for restricting the positional relation between the main body 31 of the sheath 30 and the needle body 35 in an assembled state of them, and an unlocked state (see FIGS. 38, 39, 44, and 45) for releasing the locked state.

As depicted in FIGS. 35 and 38, the locking section 12 can include projections 357 as first engaging sections formed to project on the needle body 35 (one member) (of the main body 31 of the sheath 30 and the needle body 35), and recesses 324 as second engaging sections provided in the main body 31 (the other member).

The recesses 324 are provided in the distal separable piece 32 constituting the main body 31, and two recesses 324 are disposed opposite to each other with a center axis of the distal separable piece 32 therebetween. Note that the recesses 324 may penetrate the distal separable piece 32 in the thickness direction of the distal separable piece 32, or may be recessed to an intermediate portion in the thickness direction of the distal separable piece 32.

The projections 357 are provided one on each of the elastic pieces 356. As the elastic pieces 356 are elastically deformed, the respective projections 357 can be brought closer to or away from the corresponding recesses 342.

The locking section 12 assumes the locked state in a condition where the projections 357 have been brought closer to and fitted into the recesses 324, namely, where the projections 357 and the recesses 324 are engaged with each other, as depicted in FIGS. 35 to 37. In this locked state, the positional relation between the main body 31 of the sheath 30 and the needle body 35 in the assembled state is restricted, whereby the needle body 35 can be prevented from being drawn out of the main body 31 of the sheath 30.

In accordance with an exemplary embodiment, the locking section 12 assumes the unlocked state in a condition where the projections 357 have been spaced and drawn out of the recesses 324, namely, where the projections 357 and the recesses 324 are disengaged from each other, as depicted in FIGS. 38, 39, 44, and 45. In this unlocked state, the restriction on the positional relation is released, so that the needle body 35 can be drawn out of the main body 31 of the sheath 30.

Note that the locking section 12 is a mechanism having the projections 357 provided on the needle body 35 and having the recesses 324 provided in the main body 31 in the present embodiment, this is not restrictive. The locking section 12 may be a mechanism having projections provided on the main body 31 and having recesses in the needle body 35.

As aforementioned, the elastic pieces 356 are biased toward each other by their own elastic forces. As a result, the projections 357 are biased in a direction for spacing away from the recesses 324 by the biasing forces of the elastic pieces 356 themselves. By this, the unlocked state can be reliably established. Thus, the elastic pieces 356 functions as a biasing section for biasing the projections 357 in the direction for spacing away from the recesses 324.

In addition, the insertion section 71 of the operating member 7 presses the projections 357 toward the recesses 324 against the biasing forces of the elastic pieces 356, in the state of being inserted in and engaged with the engaging section 353 of the needle body 35. By this, the locked state can be assuredly established. Thus, the insertion section 71 functions also as a pressing member. Drawing the insertion section 71 out of the engaging section 353 starting from the locked state results in the unlocked state.

As described above, in the puncture apparatus 1, the elastic pieces 356 and the insertion section 71 constitute an operating mechanism for reliably performing an operation to bring the projections 357 closer to and away from the recesses 324.

As illustrated in FIGS. 35 to 47C, during puncture by the puncture member 3, the locking section 12 is in the aforementioned locked state. This locked state can help ensure that as the needle body 35 is advanced in the distal direction, the main body 31 can also be reliably moved in the same direction. Consequently, a puncture hole can be assuredly formed in a living body.

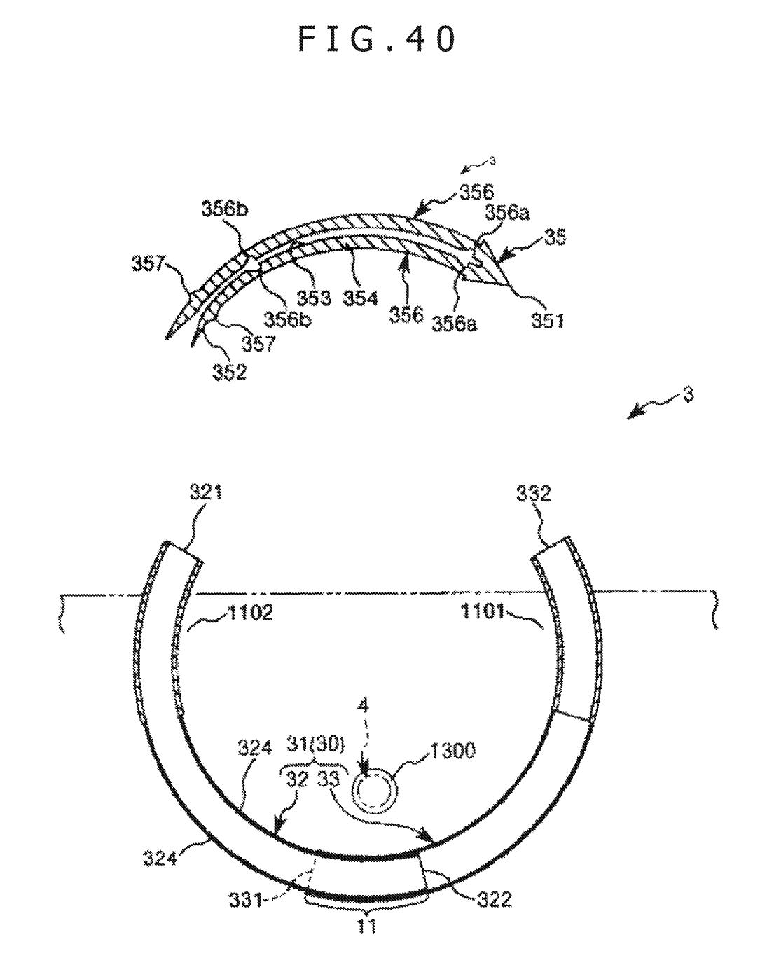

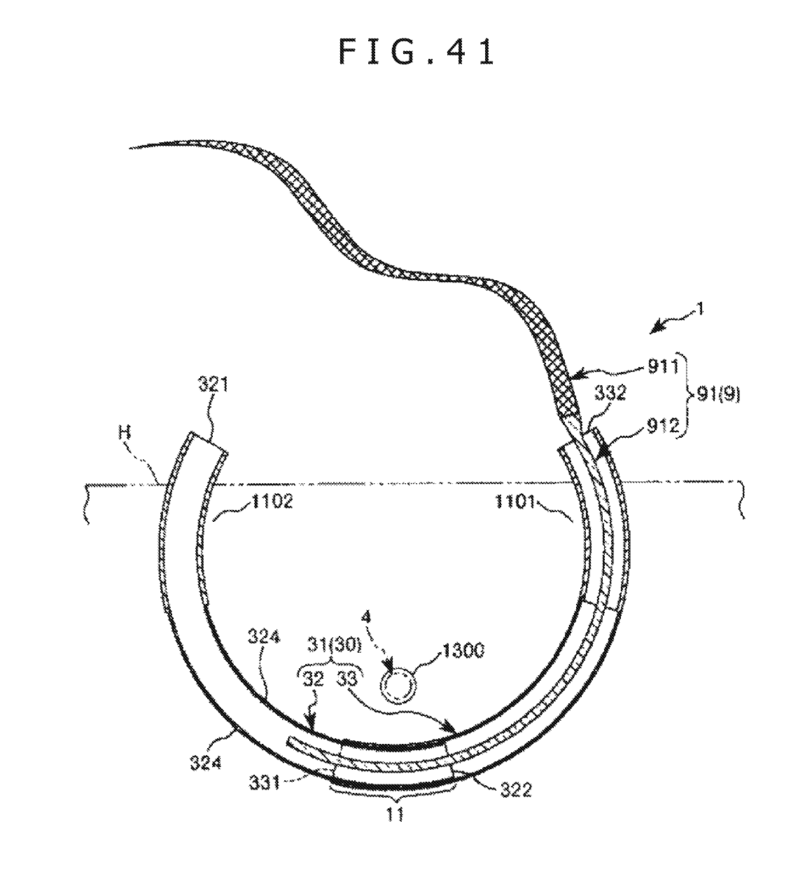

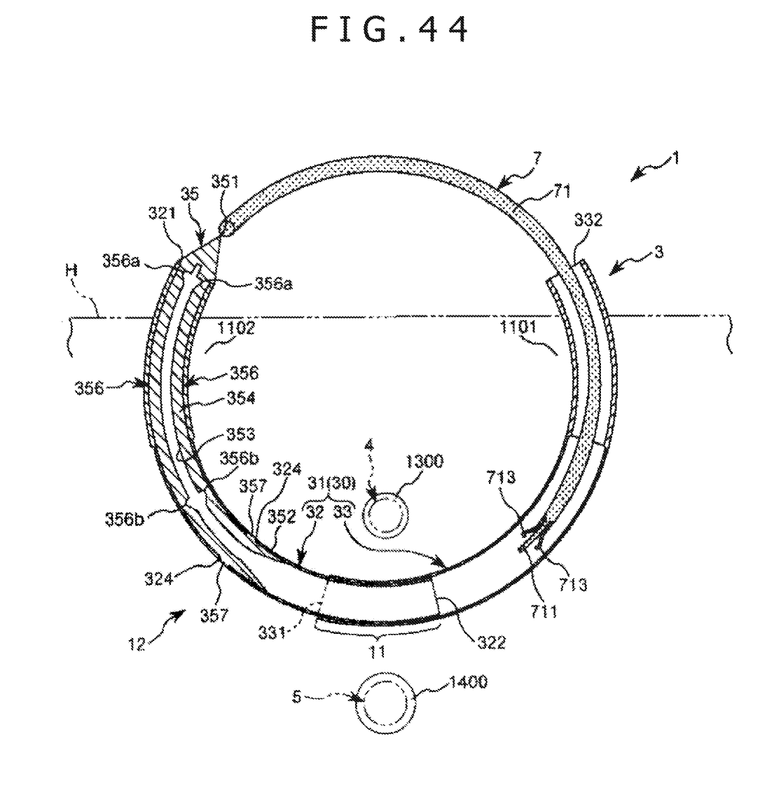

When the insertion section 71 is drawn out of the puncture member 3 after completion of the puncture, as depicted in FIG. 38 (also in FIG. 44), the locking section 12 is set into the aforementioned unlocked state. This unlocked state help ensure that the needle body 35 having been in the assembled state with the main body 31 can be drawn out of the main body 31 relatively easily and reliably, as illustrated in FIG. 40. Thereafter, the implant 9 can be inserted into the main body 31, as depicted in FIG. 41.

At a distal portion of the insertion section 71 of the operating member 7, there are provided a pair of engaging pieces 713 which are elastically deformable. In accordance with an exemplary embodiment, the engaging pieces 713 are each supported on an outer circumferential portion of the insertion section 71 in a cantilever fashion, and are protruding to opposite sides.

Each of the elastic pieces 356 of the needle body 35 is provided with a first engaging portion 356a and a second engaging portion 356b with which the engaging piece 713 of the insertion section 71 engages at different timings. The first engaging portion 356a and the second engaging portion 356b are located at different positions in the longitudinal direction of the engaging piece 713; specifically, for example, the first engaging portion 356a is located on the distal side, and the second engaging portion 356b is located on the proximal side. The first engaging portions 356a and the second engaging portions 356b are each composed of a recess formed to be recessed in the thickness direction of the elastic piece 356.

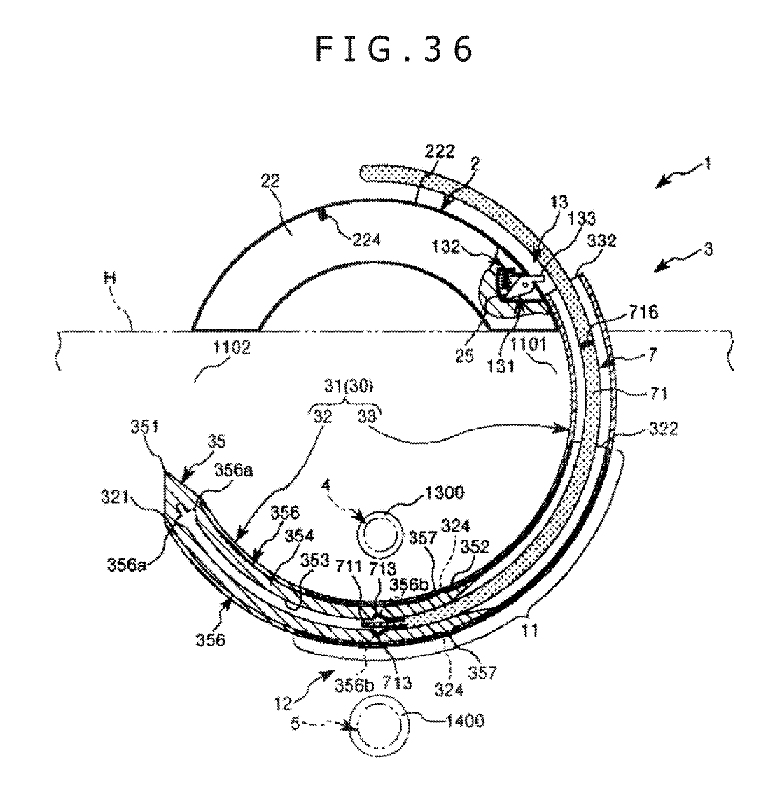

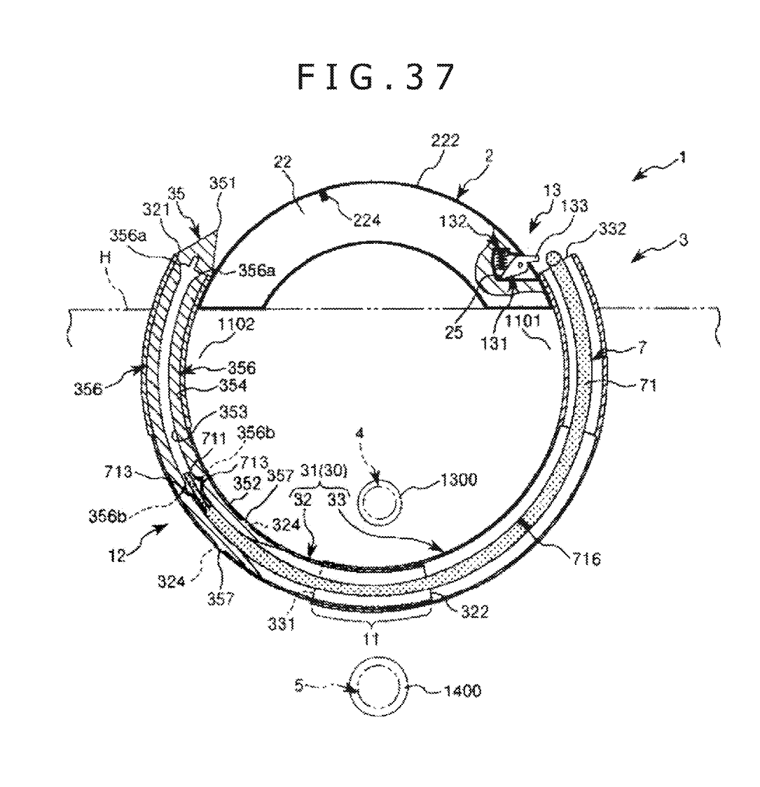

After the puncture by the puncture member 3 is started and until the state depicted in FIG. 35 is established, each of the engaging pieces 713 of the insertion section 71 is in engagement with the first engaging portion 356a of the needle body 35. Then, as depicted in FIG. 36, when the operating member 7 is rotated counterclockwise in the figure to evacuate the insertion section 71 in the proximal direction, each of the engaging pieces 713 of the insertion section 71 is disengaged from the first engaging portion 356a and is engaged with the second engaging portion 356b. When the operating member 7 is again rotated clockwise conversely to the above, starting from the state depicted in FIG. 36, the insertion section 71 advances in the distal direction, as depicted in FIG. 37, whereby the needle body 35 can be pushed out in the distal direction together with the distal separable piece 32. By this, the distal separable piece 32 can be moved in the distal direction in relation to the proximal separable piece 33, so that the overall length of the main body 31 is extended. Thus, in the present embodiment, the insertion section 71 functions also as an operating mechanism which actuates the extension section 11 to operate in the locked state.

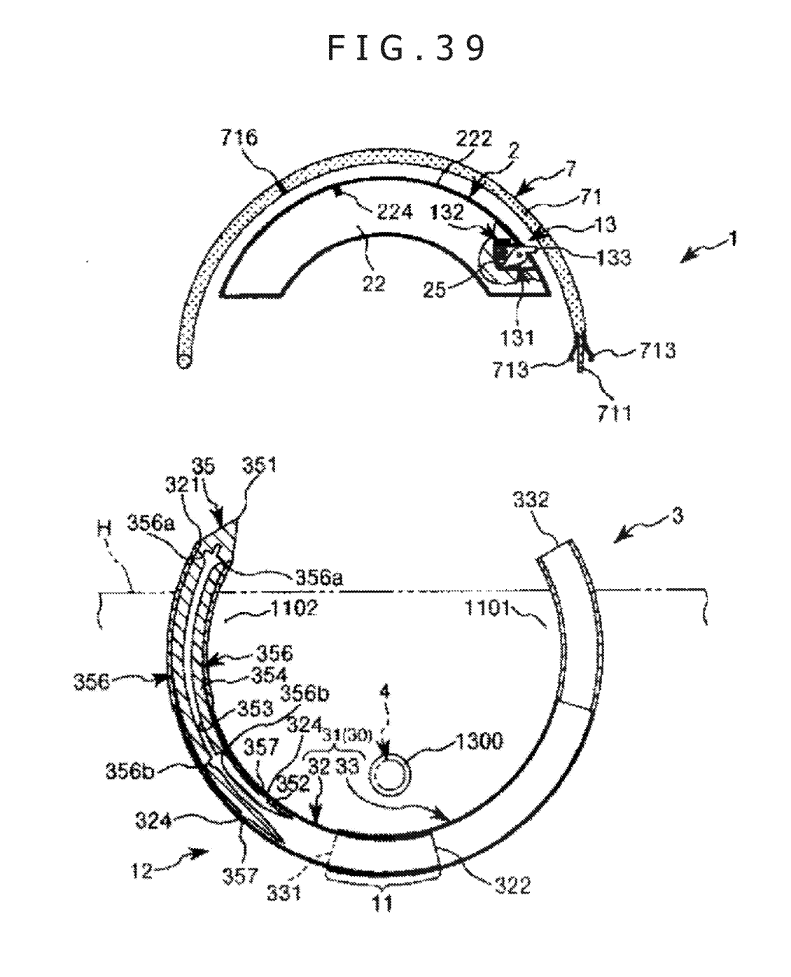

When the insertion section 71 is evacuated in the proximal direction in the same manner as aforementioned, after the extension of the main body 31, the insertion section 71 is disengaged from the second engaging portion 356b, whereby its interlock with the needle body 35 being in the assembled state with the main body 31 is canceled, as illustrated in FIG. 38. By this, the insertion section 71 can be drawn out of the puncture member 3, as depicted in FIG. 39.

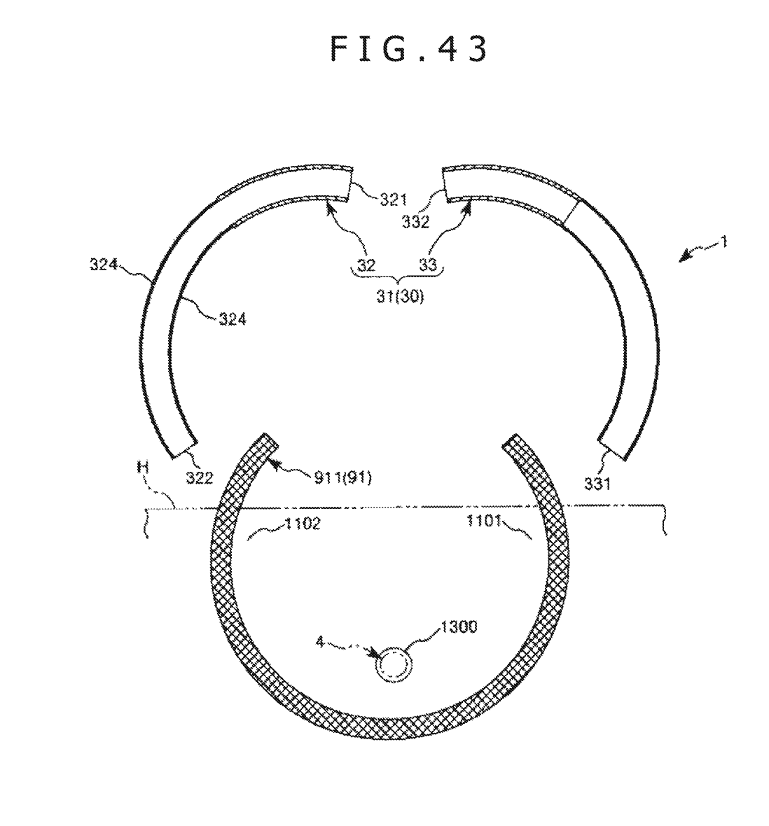

As depicted in FIG. 43, the distal separable piece 32 and the proximal separable piece 33 can be separated from each other after the insertion section 71 is drawn out of the main body 31 after the extension. This configuration will be described.

As illustrated in FIGS. 46A and 46B, the proximal separable piece 33, out of the distal separable piece 32 and the proximal separable piece 33, is provided with projections 334, whereas the distal separable piece 32 is provided with recesses 325.

The projections 334, the number of which is two, are provided on an outer circumferential portion of the proximal separable piece 33, and are protruding in opposite directions. Parts near the projections 334 of the proximal separable piece 33 can be biased in directions for spacing away from the recesses 325 by elastic forces of the proximal separable piece 33 itself.

The recesses 325, the number of which is two, are disposed opposite to each other with the center axis of the distal separable piece 32 therebetween. Note that the recess 325 may penetrate the distal separable piece 32 in the thickness direction of the distal separable piece 32, or may be recessed to an intermediate portion in the thickness direction of the distal separable piece 32.

In a state where the insertion section 71 is inserted in the main body 31, as illustrated in FIG. 46A, the insertion section 71 presses the projections 334 toward the sides of the recesses 325 against the elastic forces of the proximal separable piece 33. By this, the projections 334 can be reliably engaged with the recesses 325, and the engaged state is maintained. Note that this state is a state after the extension.

In a state where the insertion section 71 has been drawn out of the main body 31, as depicted in FIG. 46B, the pressing forces on the projections 334 are released. As a result, a restoring force of the proximal separable piece 33 is generated, whereby the projections 334 are disengaged from the recesses 325, or are evacuated. For example by pulling the distal separable piece 32 in the distal direction in this state, the distal separable piece 32 and the proximal separable piece 33 are separated from each other, so that these separable pieces can be drawn out of the living body, as depicted in FIG. 43.

Note that in the state where the insertion section 71 has been drawn out of the main body 31, the locking section 12 is in the aforementioned unlocked state. In this instance, the main body 31 can be divided into the distal separable piece 32 and the proximal separable piece 33. Thus, in the puncture apparatus 1, the locking section 12 serves also for an operation to divide the main body 31 at the time of getting into the unlocked state. Therefore, it is unnecessary to separately providing a mechanism for the dividing operation, which contributes to a simplified structure of the puncture apparatus 1.

In addition, while the projections 334 are provided on the proximal separable piece 33 and the recesses 325 are provided in the distal separable piece 32 in the present embodiment, this is not restrictive. Projections may be provided on the distal separable piece 32, and recesses may be provided in the proximal separable piece 33.

As illustrated in FIGS. 35 to 39, at the guide section 22 of the frame 2, there is disposed a movement preventing mechanism 13. The movement preventing mechanism 13 is a mechanism for preventing the main body 31 from moving together with the insertion section 71 of the operating member 7 when the insertion section 71 is evacuated in the proximal direction (see FIG. 36) or when the insertion section 71 is drawn out of the main body 31 (see FIG. 38).

The movement preventing mechanism 13 can include a stopper 131 supported on the frame 2 in a rotationally movable manner, and a compression coil spring (hereinafter referred to simply as “coil spring”) 132 as a biasing member for biasing the stopper 131.

In accordance with an exemplary embodiment, the stopper 131 is accommodated in a recess 25 opening to the guide groove 222 of the frame 2. The stopper 131 has a protruding piece 133 capable of protruding and retracting in relation to the guide groove 222 in response to rotary movement. In a state where the protruding piece 133 is protruding into the guide groove 222, as depicted in FIGS. 36 to 38, the proximal-side opening 332 of the main body 31 can engage the protruding piece 133, whereby a further proximal movement of the main body 31 is prevented, which can help ensure that, for example at the time of drawing the operating member 7 out of a living body, the puncture member 3 (the main body 31) can be securely prevented from being drawn out together with the operating member 7. In addition, in a state where the protruding piece 133 is retracted into the recess 25 and evacuated from the guide groove 222, as depicted in FIG. 35, the protruding piece 133 is prevented from obstructing the passage of the puncture member 3 through the guide groove 222.

The coil spring 132 is accommodated in the recess 25 together with the stopper 131 in a compressed state, and is disposed on the opposite side of a rotary movement axis of the stopper 131 from the protruding piece 133. This structure can help enable the coil spring 132 to bias the stopper 131 in such a direction that the protruding piece 133 protrudes into the guide groove 222.

Now, a method of using the puncture apparatus 1 (a puncturing method) according to the present embodiment will be described below, referring to FIGS. 35 to 43.

The puncture apparatus 1 is set into the state depicted in FIG. 12, in the same manner as in the method of using the puncture apparatus 1 in the first embodiment above. Thereafter, as depicted in FIG. 35, the operating member 7 is rotated clockwise. By this, the needle body 35 of the puncture member 3 is caused to puncture a body surface H at an inguinal region on the right side of the patient or at a neighboring part, to enter the patient's body together with the main body 31 having an overall length not yet extended, then pass an obturator foramen 1101 on one side, and pass between the urethra 1300 and the vagina 1400. Note that the rotating operation of the operating member 7 is performed to a limit position to which the needle body 35 can puncture the living body, in the state where the main body 31 is not extended.

In addition, attendant on this rotation, the main body 31 of the puncture member 3 can move within the guide groove 222 of the frame 2 while pressing down the protruding piece 133 of the stopper 131 of the movement preventing mechanism 13 against the biasing force of the coil spring 132.

In addition, in this instance, the locking section 12 is in the locked state, and the engaging pieces 713 of the insertion section 71 of the operating member 7 are in engagement with the first engaging portions 356a of the needle body 35.

When the above-mentioned limit position is reached, the protruding piece 133 of the stopper 131 is released from the pressing by the main body 31 of the puncture member 3, to be protruded into the guide groove 222 by the biasing force of the coil spring 132. After this protrusion is confirmed, the operating member 7 is rotated counterclockwise, reversely to the puncturing direction, to a rotational limit. By this, the engaging pieces 713 of the insertion section 71 of the operating member 7 are disengaged from the first engaging portions 356a of the needle body 35, and, while temporarily getting over the second engaging portions 356b, are moved to a position between the second engaging portions 356b and the proximal ends of the elastic pieces 356, in other words, to such an extent as not to be disengaged from the needle body 35. Note that the rotational limit regarding the counterclockwise rotation in this instance can be grasped by visually confirming that the proximal portion of the insertion section 71 coincides with a marker 224 given to the guide section 22 of the frame 2 or that a marker 716 given to an intermediate portion in the longitudinal direction of the insertion section 71 coincides with the proximal-side opening 332 of the main body 31.

As the operating member 7 rotates counterclockwise, the main body 31 may seem to move together with the insertion section 71. However, since the protruding piece 133 of the stopper 131 is protruding into the guide groove 222 as aforementioned, movement of the main body 31 can be inhibited by the protruding piece 133.

Next, as depicted in FIG. 36, the operating member 7 is again rotated clockwise. By this, the engaging pieces 713 of the insertion section 71 of the operating member 7 can engage the second engaging portions 356b which the engaging pieces 713 have once gotten over as above-mentioned.

Then, as depicted in FIG. 37, the operating member 7 is again rotated clockwise directly and in a continued manner. In this instance, since the locked state of the locking section 12 is maintained, the needle body 35 together with the distal separable piece 32 can be pressed in the distal direction. By this, the main body 31 is extended, and the needle body 35 is caused to pass an obturator foramen 1102 on the other side, and exit the patient's body via the body surface H at an inguinal region on the left side or at a neighboring part (second part).

Note that when the main body 31 is extended, the projections 334 of the proximal separable piece 33 are engaged with the recesses 325 of the distal separable piece 32, whereby further extension is restricted.

Subsequently, as depicted in FIG. 38, the operating member 7 is again rotated counterclockwise, and the insertion section 71 is thereby drawn out of the needle body 35. This puts the locking section 12 into the unlocked state, as aforementioned.

Note that by causing the proximal portion of the insertion section 71 to coincide with the marker 224, a state is established in which the engaging pieces 713 of the insertion section 71 are disengaged from the second engaging portions 356b and the insertion section 71 is drawn out of the needle body 35.

When the insertion section 71 is drawn out of the needle body 35 and passes through the extension section 11, the projections 334 of the proximal separable piece 33 are disengaged from the recesses 325 of the distal separable piece 32, as aforementioned. This results in a state in which the distal separable piece 32 and the proximal separable piece 33 are separable.

Next, as illustrated in FIG. 39, the operating member 7 is removed from the living body together with the frame 2, while leaving the puncture member 3 inside the living body.

Subsequently, as depicted in FIG. 40, the needle body 35 is drawn out of the main body 31. This drawing-out operation is possible because the locking section 12 is in the unlocked state.

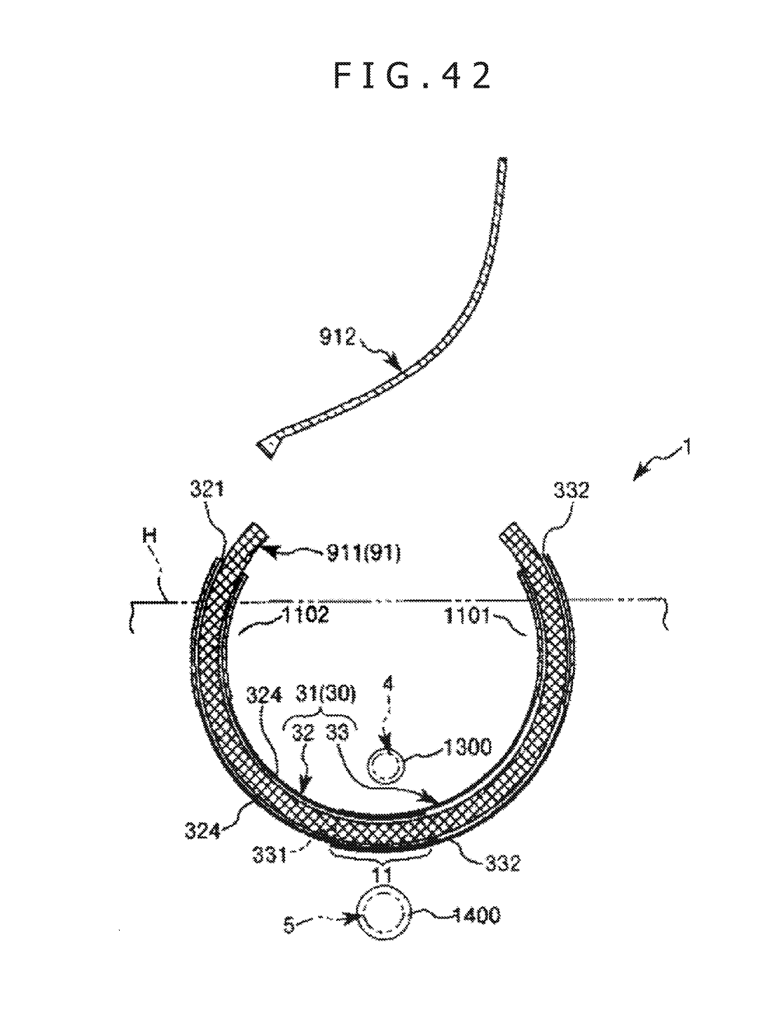

Next, as depicted in FIG. 41, the implant main body 91 is gradually inserted into the main body 31, starting from the ribbon 912 side. Then, when the main body section 911 of the implant main body 91 have protruded from both ends of the main body 31, as depicted in FIG. 42, the ribbon 912 is cut.

Subsequently, as illustrated in FIG. 43, the distal separable piece 32 and the proximal separable piece 33 are pulled in opposite directions, to be drawn out of the living body. Thereafter, the implant main body 91 is placed indwelling, in the same manner as in the method of using the puncture apparatus 1 in the first embodiment above.

In this way, according to the puncture apparatus 1, at the time of placing the implant 9 indwelling in a living body, the main body 31 can be put into the extended state prior to the placement. Both ends of the main body 31 in the extended state can be reliably exposed from the body surface H. Then, after temporarily inserting the implant 9 via the thus exposed part, placement of the implant 9 indwelling can be carried out.

In addition, by unlocking the locking section 12, the needle body 35 can be drawn out of the sheath 30 easily and assuredly, while leaving the sheath 30 indwelling in the living body after puncturing of the living body, which can help enable swift transition to the operation of inserting the implant 9 after the drawing-out of the needle body 35 in the unlocked state.

In addition, after the needle body 35 is drawn out, the main body 31 can be divided into the distal separable piece 32 and the proximal separable piece 33, which can help enable the implant 9 to be quickly placed indwelling.

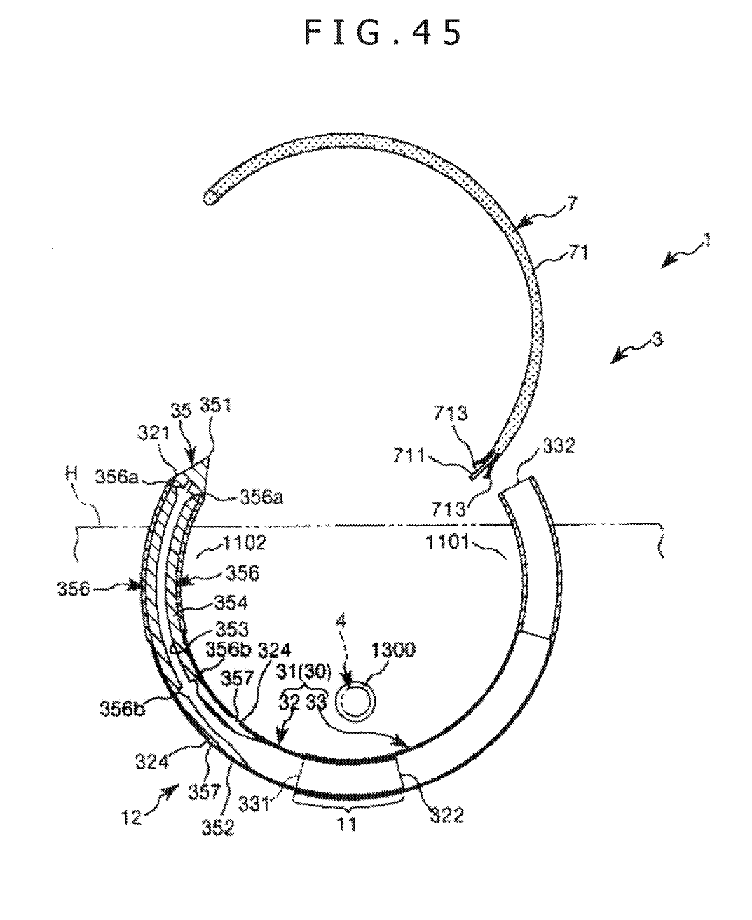

Now, another operating method for the puncture apparatus 1 in the present embodiment will be described referring to FIGS. 44 and 45.

The frame 2 can be removed from the living body, while leaving the puncture member 3 and the operating member 7 inside the living body, after the extension of the main body 31, as illustrated in FIG. 44.

Thereafter, the operating member 7 can be drawn out of the puncture member 3, to be removed from the living body, while leaving the puncture member 3 inside the living body, as depicted in FIG. 45.

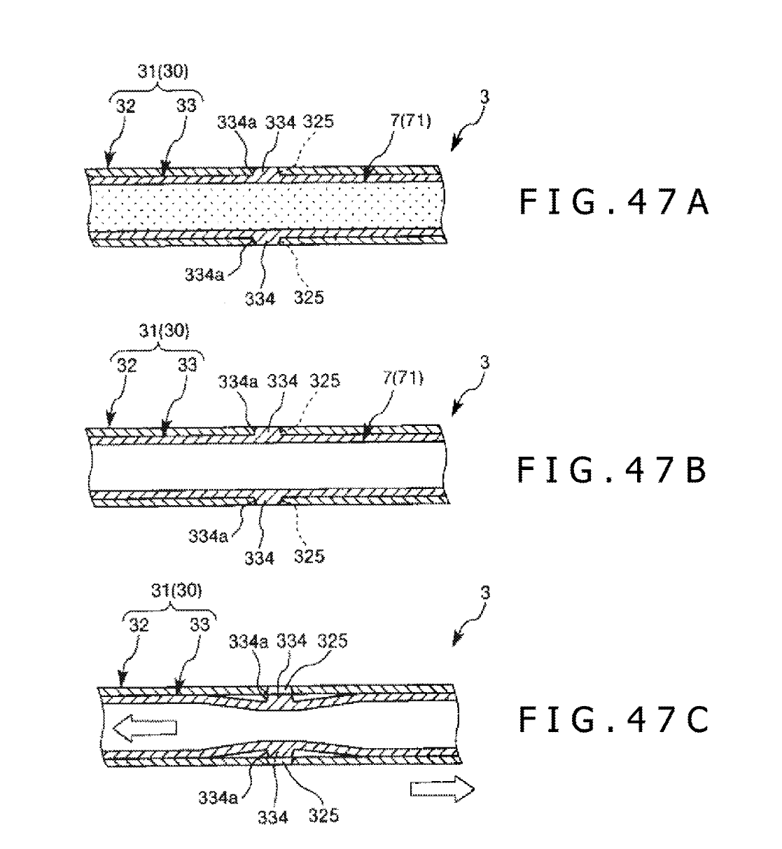

FIGS. 47A to 47C are longitudinal sectional views of a medical tube according to a tenth embodiment of the present disclosure.

Referring to these figures, the tenth embodiment of the medical tube, medical a device set, and a method of placing an implant indwelling according to the present disclosure will be described below. In the following, description will be made primarily of differences from the aforementioned embodiments, and descriptions of the same items as above will be omitted.

The tenth embodiment is the same as the ninth embodiment above, except for differences in the configuration of sheath.

In the present embodiment as illustrated in FIGS. 47A to 47C, a proximal separable piece 33 constituting a main body 31 of a sheath 30 is not configured so that parts near the projections 334 of the proximal separable piece 33 are biased toward the recesses 325 by elastic forces of the proximal separable piece 33 itself. A method of separating the distal separable piece 32 and the proximal separable piece 33 from each other in the case of such a configuration will be described.

In a state where the insertion section 71 is inserted in the main body 31, as illustrated in FIG. 47A, the insertion section 71 is pressing the projections 334 toward the recesses 325. By this, the projections 334 can be reliably engaged with the recesses 325, and the engaged state can be maintained.

Then, the insertion section 71 is drawn out of the main body 31, as depicted in FIG. 47B. In this state, the pressing forces on the projections 334 are released.

Next, the distal separable piece 32 and the proximal separable piece 33 are pulled in opposite directions, as depicted in FIG. 47C. In this instance, inclined surfaces 334a as side surfaces of the projections 334 enable the projections 334 to get over the recesses 325, to be disengaged from the recesses 325. Consequently, the distal separable piece 32 and the proximal separable piece 33 can be separated from each other and each can be drawn out of the living body.

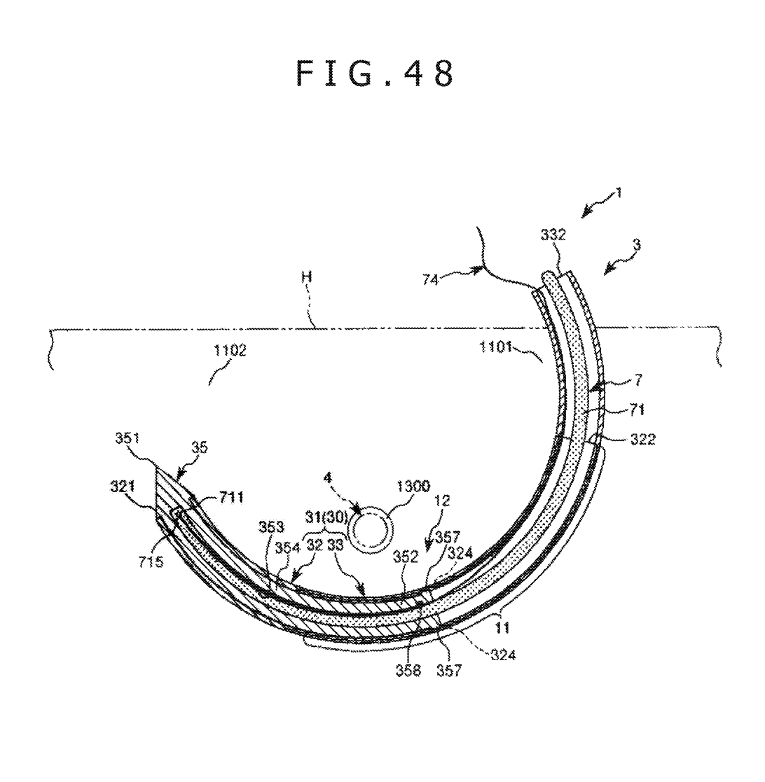

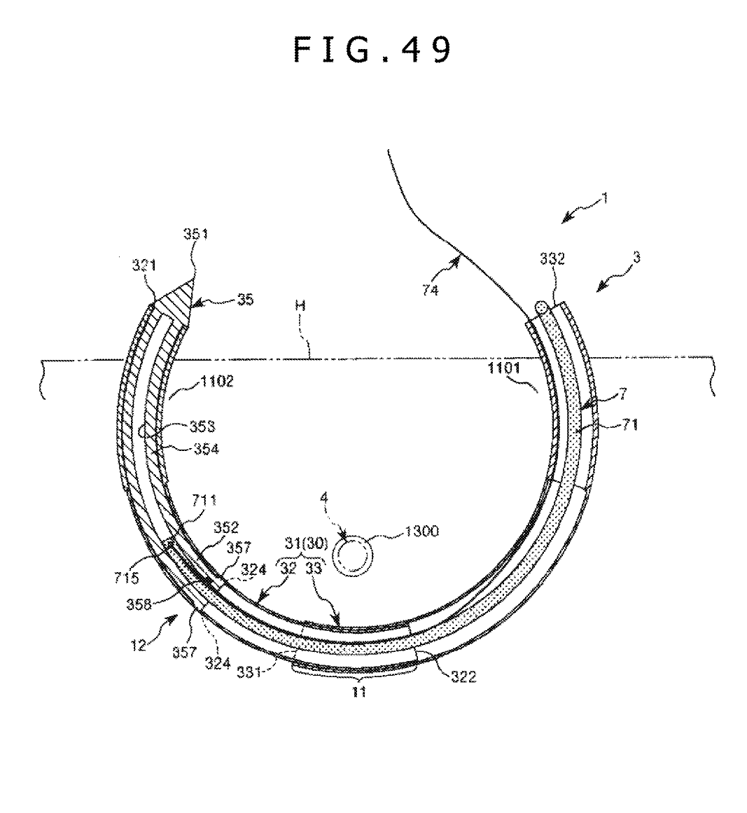

FIGS. 48 and 49 are views (as viewed from the side of the patient's legs) for explaining an operating procedure of a puncture apparatus in the case where a medical tube according to an eleventh embodiment of the present disclosure is applied to the puncture apparatus.

Referring to these figures, the eleventh embodiment of the medical tube, a medical device set, and a method of placing an implant indwelling according to the present disclosure will be described below. In the following, description will be made primarily of differences from the aforementioned embodiments, and descriptions of the same items as above will be omitted.

The eleventh embodiment is the same as the ninth embodiment, except for differences in the configuration of operating member.

As illustrated in FIGS. 48 and 49, in the present embodiment, an operating member 7 has a distal portion 711 formed therein with a through-hole 715. A string 74 inserted in a main body 31 is inserted into and passed through the through-hole 715.

The string 74 functions as an operating mechanism for operating the extension section 11, specifically, for an operation to extend the main body 31. The string 74 can be gripped at its one-end portion exposed from the proximal-side opening 332 of the main body 31. The other-end portion of the string 74 is inserted into and passed through the through-hole 715, is folded back in the through-hole 715, and is fixed to a proximal section 352 of the needle body 35 through a fixing portion 358.

A method of using the puncture apparatus 1 having the operating member 7 configured in this manner will be described.

As depicted in FIG. 48, the operating member 7 is rotated clockwise. By this, the needle body 35 of the puncture member 3 is caused to puncture a body surface H at an inguinal region on the right side of the patient or at a neighboring part, to enter the patient's body together with the main body 31 being in the state of having an overall length not yet extended, pass an obturator foramen 1101 on one side, and pass between the urethra 1300 and the vagina 1400. Note that the rotating operation of the operating member 7 is conducted to a limit position to which the needle body 35 can puncture the living body, in the state where the main body 31 is not extended.

Next, the one-end side portion of the string 74 is gripped, and is pulled directly. By this pulling operation, the distance between the through-hole 715 of the insertion section 71 and the fixing portion 358 of the needle body 35 is reduced by the pulling distance of the string 74, as depicted in FIG. 49. As a result, the needle body 35 is moved in the distal direction together with the main body 31, so that the main body 31 is extended. Note that the operation of pulling the string 74 is performed until the needle body 35 protrudes from the body surface H to the outside of the body.

Subsequently, the hand having gripped the string 74 is let off, and the insertion section 71 of the operating member 7 is drawn out of the main body 31. By this, the string 74 is drawn out of the through-hole 715 of the insertion section 71. Note that the other-end portion of the string 74 remains fixed to the fixing portion 358 of the needle body 35.

Thereafter, the needle body 35 is drawn out of the main body 31 together with the string 74. Subsequently, the same operations as in the eighth embodiment above are conducted, to place the implant 9 indwelling in the body.

Thus, in the present embodiment, the main body 31 can be extended by the simple operation of pulling the string 74, which contributes to shortening of the time taken for the procedure.

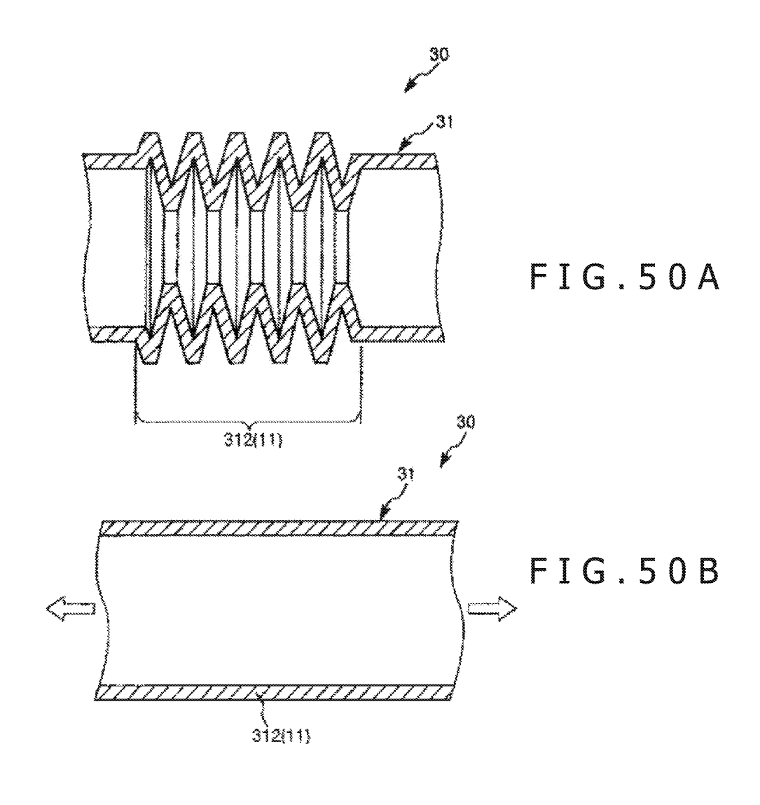

FIGS. 50A and 50B are longitudinal sectional views of a medical tube according to a twelfth embodiment of the present disclosure.

Referring to these figures, the twelfth embodiment of the medical tube, a medical device set, and a method of placing an implant indwelling according to the present disclosure will be described below. In the following, description will be made primarily of differences from the aforementioned embodiments, and descriptions of the same items as above will be omitted.

The twelfth embodiment is the same as the eighth embodiment above, except for differences in the configuration of extending mechanism.

As depicted in FIGS. 50A and 50B, in the present embodiment, a main body 31 of a sheath 30 is provided with a deformation section 312 which is deformed so as to extend along a longitudinal direction of the main body 31. Note that a part at which the deformation section 312 is disposed may be any intermediate part in the longitudinal direction of the main body 31.

The deformation section 312 is bellows-like in shape, and can assume a contracted state of being contracted as depicted in FIG. 50A and an extended state of being extended as depicted in FIG. 50B, each of the deformed states being maintained. The main body 31 can be extended by a transition of the deformation section 312 from the contracted state to the extended state.

Thus, in the present embodiment, the deformation section 312 functions as the extension section 11, which helps enable the extension section 11 to be simple in configuration.

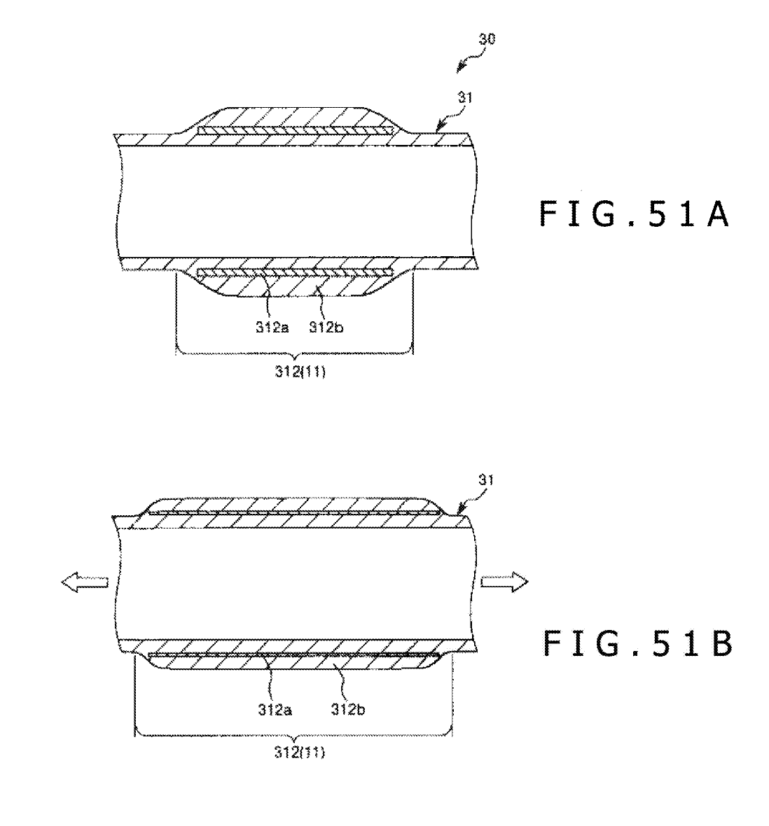

FIGS. 51A and 51B are longitudinal sectional views of a medical tube according to a thirteenth embodiment of the present disclosure.

Referring to these figures, the thirteenth embodiment of the medical tube, a medical device set, and a method of placing an implant indwelling according to the present disclosure will be described below. In the following, description will be made primarily of differences from the aforementioned embodiments, and descriptions of the same items as above will be omitted.

The thirteenth embodiment is the same as the twelfth embodiment as above, except for differences in the configuration of extension mechanism.

As illustrated in FIGS. 51A and 51B, in the present embodiment, a deformation section 312 functioning as the extension section 11 can include a thick portion 312b where the wall thickness of the main body 31 is enlarged, and a reinforcement 312a concentrically embedded in the thick portion 312b. The reinforcement 312a is composed of a metallic tube which is plastically deformable. The reinforcement 312a is mesh-like in form, and can be contracted and expanded in the axial direction and the radial direction of the tube.

When the main body 31 is pulled to the left and right sides in FIG. 51A starting from the contracted state depicted in the figure, the deformation section 312 is put into an extended state depicted in FIG. 51B. In the extended state depicted in FIG. 51B, the thick portion 312b is enlarged in overall length and reduced in thickness, by amounts corresponding to the pulling distance. Similarly, the reinforcement 312a is also enlarged in overall length and reduced in thickness. Since the reinforcement 312a is a plastically deformable member, the reinforcement 312a can maintain the state after deformation of the thick portion 312a.

In addition, as depicted in FIGS. 51A and 51B, a lumen portion of the main body 31 can be secured to be large enough to insert the implant 9 therein, before and after the deformation of the deformation section 312.

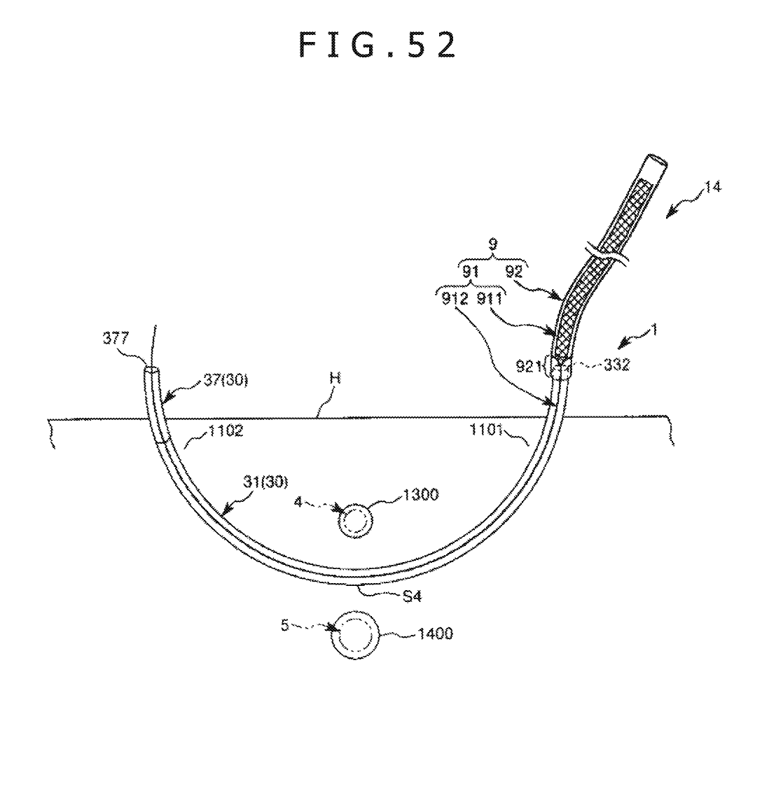

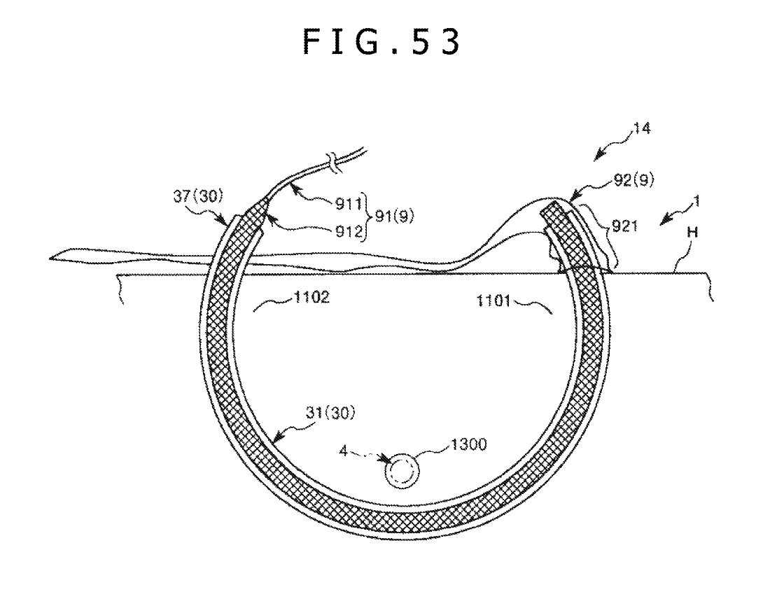

FIGS. 52 and 53 are views (as viewed from the side of the patient's legs) for explaining an operating procedure of a puncture apparatus in the case where a medical tube according to a fourteenth embodiment of the present disclosure is applied to the puncture apparatus.

Referring to these figures, the fourteenth embodiment of the medical tube and a method of placing an implant indwelling according to the present disclosure will be described below. In the following, description will be made primarily of differences from the aforementioned embodiments, and descriptions of the same items as above will be omitted.

The fourteenth embodiment is the same as the first embodiment, except for differences in the configuration of implant.

As illustrated in FIGS. 52 and 53, in the present embodiment, a distal portion of a wrapping material 92 of an implant 9 is a joint section 921 which can be joined to a proximal portion of the main body 31 of the sheath 30. In accordance with an exemplary embodiment, a proximal portion of the main body 31 of the sheath 30 is also a joint section. The inside diameter of the joint section 921 is set to be equal to or slightly greater than the outside diameter of the proximal portion of the main body 31. For this reason, in a joined state where the proximal portion of the main body 31 is joined to the joint section 921, the proximal portion of the main body 31 is located inside the joint section 921, specifically, fitted in the joint section 921. In addition, in the joined state, the wrapping material 92 and the main body 31 communicate with each other through the joint section 921.

In the present embodiment, the implant 9 and the sheath 30 (main body 31) constitute a medical assembly 14.

Note that when obtaining the joined state, prior to the joining operation, the ribbon 912 of the implant main body 91 is inserted via the proximal-side opening 332 of the main body 31, and is protruded from the distal-side opening 377 of the extension tube 37. Thereafter, the joined state is established.

Alternatively, after the joined state is obtained, the ribbon 912 of the implant main body 91 is inserted via the proximal-side opening 332 of the main body 31, and is protruded from the distal-side opening 377 of the extension tube 37.

Then, while keeping the joined state, the ribbon 912 protruding from the distal-side opening 377 is pulled, as depicted in FIG. 53, whereby the main body section 911 of the implant main body 91 is transferred (moved and inserted) from inside the wrapping material 92 into the main body 31 easily and reliably.

By the implant inserting method as above, it is possible, at the time of inserting the implant main body 91 into the sheath 30, to put the sheath 30 and the wrapping material 92 into the joined state through the joint section, prior to the insertion. Where the inserting operation is conducted in the joined state, the implant main body 91 can be securely protected from the outside air during the insertion process. As a result, the implant main body 91 can be placed indwelling in a living body while being kept as clean as possible.

In addition, the main body 31 may be configured to be separable, in the same manner as in the ninth embodiment above. In this case, when a proximal portion of the main body 31 is protruded from the body surface H in the joined state and a distal portion of the extension tube 37 is protruded from the body surface H by an amount equal to the protrusion amount of the proximal portion of the main body 31, as depicted in FIG. 52, the separating position of the main body 31 located at the central portion S4 can be positioned between the urethra 1300 and the vagina 1400. Thus, the proximal portion of the main body 31 and the distal portion of the extension tube 37 which are protruded from the body surface H function as a detection section by which the separating position of the main body 31 can be detected.

Another configuration may also be used to exhibit a function as a detection section. For example, at least one marker is given to each of a proximal portion of the main body 31 and a distal portion of the extension tube 37. In accordance with an exemplary embodiment, the distance between the marker on the main body 31 side and the body surface H is equal to the distance between the marker on the extension tube 37 side and the body surface H. By such a configuration, also, the function as the detection section can be realized.

In addition, a force for dividing the main body 31 can be set to be smaller than a force for releasing the joint between the main body 31 and the extension tube 37 in the joined state, which can help ensure that when the main body 31 is divided (separated), the main body 31 can be divided reliably while preventing the extension tube 37 from being detached from the main body 31.

In addition, while the wrapping material 92 is joined to the proximal portion of the main body 31 of the sheath 30 in the present embodiment, this is not restrictive. The wrapping material 92 may be joined to a distal portion of the main body 31 of the sheath 30, and the distal portion may function as the joint section.

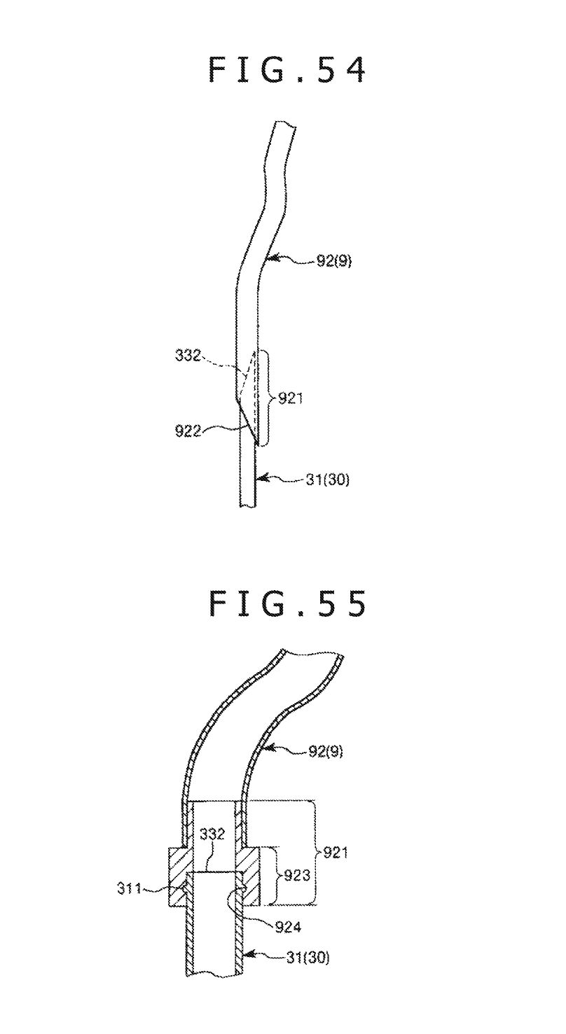

FIG. 54 is a view for explaining an operating procedure of a puncture apparatus in the case where a medical tube according to a fifteenth embodiment of the present disclosure is applied to the puncture apparatus.

Referring to this figure, the fifteenth embodiment of the medical tube, a medical device set, and a method of placing an implant indwelling according to the present disclosure will be described below. In the following, description will be made primarily of differences from the aforementioned embodiments, and descriptions of the same items as above will be omitted.

The fifteenth embodiment is the same as the fourteenth embodiment above, except for differences in the shape of wrapping material.

As illustrated in FIG. 54, in the present embodiment, a joint section 921 of a wrapping material 92 has an opening 922 which is formed to be inclined against a longitudinal direction of the wrapping material 92.

In accordance with an exemplary embodiment, a proximal-side opening 332 of a main body 31, also, is preferably, for example, formed to be inclined against a longitudinal direction of the main body 31.

In accordance with an exemplary embodiment, owing to a synergistic effect of the inclination of the opening 922 and the inclination of the proximal-side opening 332, an operation of joining the proximal-side opening 332 of the main body 31 to the joint section 921 can be carried out relatively easily.

Note that in the present embodiment, the inclination of the proximal-side opening 332 of the main body 31 is not restrictive.

FIG. 55 is a view for explaining an operating procedure of a puncture apparatus in the case where a medical tube according to a sixteenth embodiment of the present disclosure is applied to the puncture apparatus.

Referring to this figure, the sixteenth embodiment of the medical tube, a medical device set, and a method of placing an implant indwelling according to the present disclosure will be described below. In the following, description will be made primarily of differences from the aforementioned embodiments, and descriptions of the same items as above will be omitted.

The sixteenth embodiment is the same as the fourteenth embodiment above, except for differences in the configuration of wrapping material.

As depicted in FIG. 55, in the present embodiment, a joint section 921 of a wrapping material 92 is composed of a tube shaped member, which makes the joint section 921 the thickest part of the wrapping material 92, and, hence, the most rigid part of the wrapping material 92. In accordance with an exemplary embodiment, the main body 31 as a mating member to be locked to the joint section 921 is also comparatively hard, or comparatively high in rigidity. Therefore, an operation of locking the proximal-side opening 332 of the main body 31 to the joint section 921 can be carried out relatively easily.

Note that the material constituting the joint section 921 is not particularly limited. For example, the same material as that for the sheath 30 is preferably used as the material for the joint section 921.

In addition, the joint section 921 is provided at a distal portion thereof with an enlarged diameter portion 923, which is enlarged in outside diameter. In accordance with an exemplary embodiment, the enlarged diameter portion 923 can be gripped in obtaining a joined state.

In addition, the enlarged diameter portion 923 is formed in an inner circumferential portion thereof with a recess 924 along the circumferential direction thereof. In accordance with an exemplary embodiment, a main body 31 is provided at an outer circumferential portion thereof with a projection 311 along the circumferential direction thereof. In the joined state, the projection 311 engages the recess 924. By this engagement, the joined state can be assuredly maintained. Accordingly, the wrapping material 92 and the main body 31 can be prevented from being unintendedly detached from each other during transfer of the implant main body 91 from inside the wrapping material 92 into the main body 31.

While the recess 924 is provided in the wrapping material 92 and the projection 311 is provided on the main body 31 in the present embodiment, this mechanism is not restrictive. A recess may be provided in the main body 31, and a projection may be provided on the wrapping material 92.

FIG. 56 depicts a longitudinal sectional view and a cross-sectional view for explaining an operating procedure of a puncture apparatus in the case where a medical tube according to a seventeenth embodiment of the present disclosure is applied to the puncture apparatus.

Referring to this figure, the seventeenth embodiment of the medical tube and a method of placing an implant indwelling according to the present disclosure will be described below. In the following, description will be made primarily of differences from the aforementioned embodiments, and descriptions of the same items as above will be omitted.

The seventeenth embodiment is the same as the fourteenth embodiment above, except for differences in the configuration of wrapping material and/or medical tube.

As illustrated in FIG. 56, in the present embodiment, a joint section 921 of a wrapping material 92 is enlarged in diameter in a trumpet-shaped form, and can be joined to a proximal portion (joint portion) of a main body 31 in a loose fit state. In this joined state, the joint section 921 can cover the proximal portion of the main body 31, and the joined state is maintained. Consequently, the main body section 911 of the implant main body 91 can be transferred from inside the wrapping material 92 into the main body 31 in a clean state.

FIG. 57 depicts a longitudinal sectional view and a cross-sectional view for explaining an operating procedure of a puncture apparatus in the case where a medical tube according to an eighteenth embodiment of the present disclosure is applied to the puncture apparatus.

Referring to this figure, the eighteenth embodiment of the medical tube and a method of placing an implant indwelling according to the present disclosure will be described below. In the following, description will be made primarily of differences from the aforementioned embodiments, and descriptions of the same items as above will be omitted.

The eighteenth embodiment is the same as the seventeenth embodiment above, except for differences in the configuration of wrapping material and/or medical tube.

As depicted in FIG. 57, in the present embodiment, a joint section 921 of a wrapping material 92 is a part which has a whole perimeter greater than the whole perimeter of a proximal portion (joint portion) of a main body 31, is flexible, and can be joined to the proximal portion of the main body 31 in a loose fit state. In the joined state, the joint section 921 can cover the proximal portion of the main body 31, and the joined state is maintained. As a result, the main body section 911 of the implant main body 91 can be transferred from inside the wrapping material 92 into the main body 31 in a clean state.

FIG. 58 depicts a longitudinal sectional view and a cross-sectional view for explaining an operating procedure of a puncture apparatus in the case where a medical tube according to a nineteenth embodiment of the present disclosure is applied to the puncture apparatus.

Referring to this figure, the nineteenth embodiment of the medical tube and a method of placing an implant indwelling according to the present disclosure will be describe below. In the following, description will be made primarily of differences from the aforementioned embodiments, and descriptions of the same items as above will be omitted.

The nineteenth embodiment is the same as the seventeenth embodiment above, except for differences in the configuration of wrapping material and/or medical tube.

As depicted in FIG. 58, in the present embodiment, a joint section 921 of a wrapping material 92 is a part which has a whole perimeter greater than the whole perimeter of a proximal portion (joint portion) of a main body 31, is wound in a roll form, and can be joined to the proximal portion of the main body 31 in a loose fit state. In the joined state, the joint section 921 can cover the proximal portion of the main body 31, and the jointed state is maintained. Accordingly, the main body section 911 of the implant main body 91 can be transferred from inside the wrapping material 92 into the main body 31 in a clean state.

FIG. 59 depicts a longitudinal sectional view and a cross-sectional view for explaining an operating procedure of a puncture apparatus in the case where a medical tube according to a twentieth embodiment of the present disclosure is applied to the puncture apparatus.

Referring to this figure, the twentieth embodiment of the medical tube and a method of placing an implant indwelling according to the present disclosure will be described below. In the following, description will be made primarily of differences from the aforementioned embodiments, and descriptions of the same items as above will be omitted.

The twentieth embodiment is the same as the fourteenth embodiment above, except for differences in the configuration of wrapping material and/or medical tube.

As depicted in FIG. 59, in the present embodiment, a joint section 921 of a wrapping material 92 is a tube shaped part having a hardness equal to or higher than that of a main body 31, and can be joined to a proximal portion of the main body 31 by entering and fitting into the inside of the proximal portion. By this, the joined state is maintained, and, accordingly, the main body section 911 of the implant main body 91 can be transferred from inside the wrapping material 92 into the main body 31.

FIG. 60 depicts a longitudinal sectional view and a cross-sectional view for explaining an operating procedure of a puncture apparatus in the case where a medical tube according to a twenty-first embodiment of the present disclosure is applied to the puncture apparatus.

Referring to this figure, the twenty-first embodiment of the medical tube and a method of placing an implant indwelling according to the present disclosure will be described below. In the following, description will be made primarily of differences from the aforementioned embodiments, and descriptions of the same items as above will be omitted.

The twenty-first embodiment is the same as the twentieth embodiment above, except for differences in the configuration of wrapping material and/or medical tube.

As depicted in FIG. 60, in the present embodiment, a joint section 921 of a wrapping material 92 is a tube shaped part having a hardness equal to or higher than that of a main body 31, and can be joined to a proximal portion of the main body 31 by externally fitting over the proximal portion in a covering manner. Accordingly, the joined state is maintained, and, consequently, the main body section 911 of the implant main body 91 can be transferred from inside the wrapping material 92 into the main body 31 in a clean state.

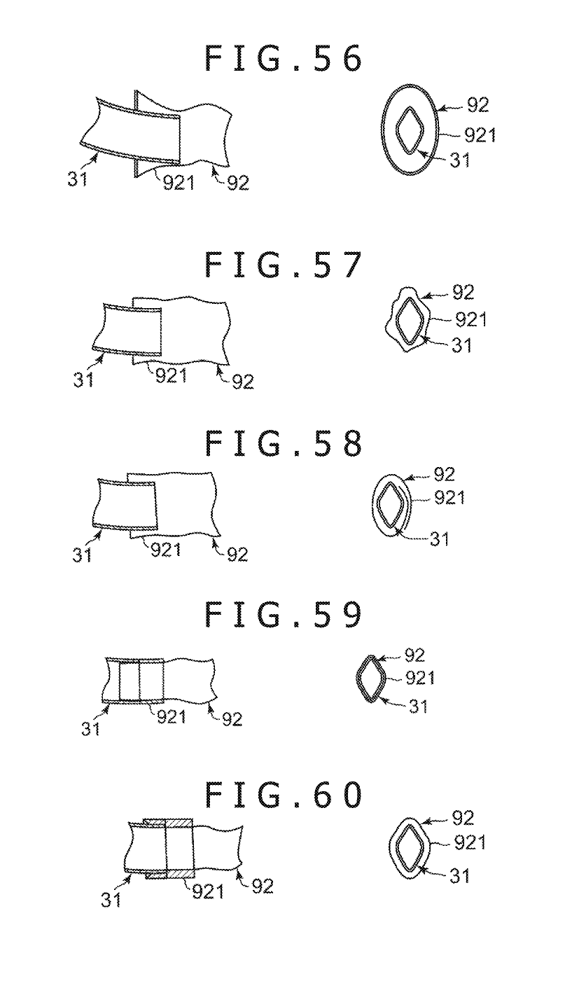

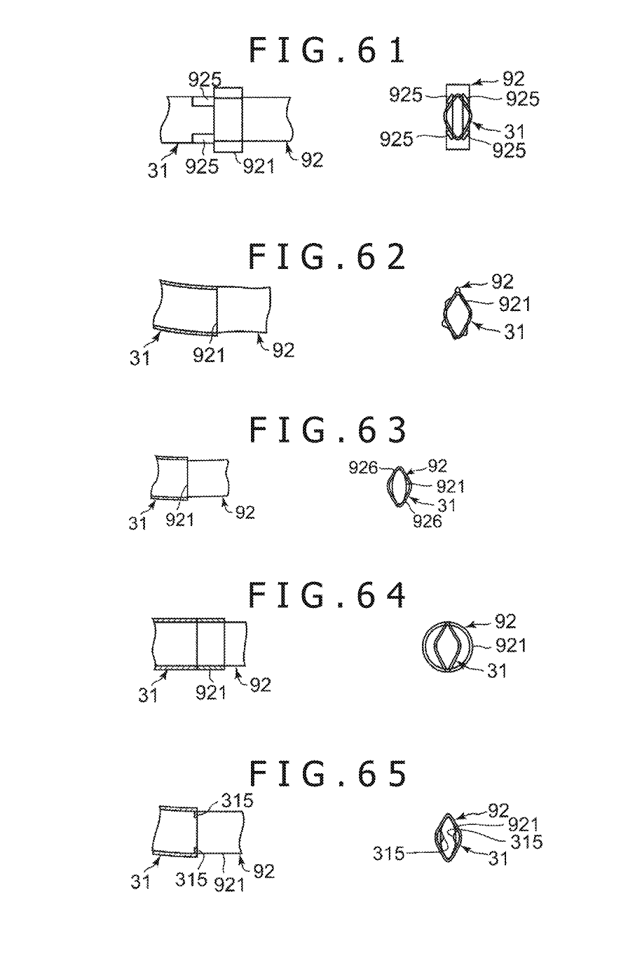

FIG. 61 depicts a longitudinal sectional view and a cross-sectional view for explaining an operating procedure of a puncture apparatus in the case where a medical tube according to a twenty-second embodiment of the present disclosure is applied to the puncture apparatus.

Referring to this figure, the twenty-second embodiment of the medical tube and a method of placing an implant indwelling according to the present disclosure will be described below. In the following, description will be made primarily of differences from the aforementioned embodiments, and descriptions of the same items as above will be omitted.

The twenty-second embodiment is the same as the twentieth embodiment above, except for differences in the configuration of wrapping material and/or medical tube.

As illustrated in FIG. 61, in the present embodiment, a joint section 921 of a wrapping material 92 is a part which has a hardness equal to or higher than that of a main body 31, and is formed with a plurality (for example, in the configuration illustrated, four) of projecting pieces 925. The joint section 921 can be joined to a proximal portion of the main body 31 by a process in which the projecting pieces 925 enter the inside of the proximal portion and bias the proximal portion outward. By this, the joined state is maintained, and, accordingly, the main body section 911 of the implant main body 91 can be transferred from inside the wrapping material 92 into the main body 31 in a clean state.

FIG. 62 depicts a longitudinal sectional view and a cross-sectional view for explaining an operating procedure of a puncture apparatus in the case where a medical tube according to a twenty-third embodiment of the present disclosure is applied to the puncture apparatus.

Referring to this figure, the twenty-third embodiment of the medical tube and a method of placing an implant indwelling according to the present disclosure will be described below. In the following, description will be made primarily of differences from the aforementioned embodiments, and descriptions of the same items as above will be omitted.

The twenty-third embodiment is the same as the fourteenth embodiment above, except for differences in the configuration of wrapping material and/or medical tube.

As illustrated in FIG. 62, in the present embodiment, a joint section 921 of a wrapping material 92 is flexible, and can be joined to a proximal surface of a main body 31, with an end surface of the joint section 921 attaching the proximal surface. By this, the joined state is maintained, and, consequently, the main body section 911 of the implant main body 91 can be transferred from inside the wrapping material 92 into the main body 31 in a clean state.

While the mode of joining between the joint section 921 of the wrapping material 92 and the proximal surface of the main body 31 is “attach” in the configuration depicted in FIG. 62, this is not restrictive, and the joining mode may be “fitting.” In this case, the wrapping material 92 may be fitted into the main body 31, or the wrapping material 92 may be fitted over the main body 31.

FIG. 63 depicts a longitudinal sectional view and a cross-sectional view for explaining an operating procedure of a puncture apparatus in the case where a medical tube according to a twenty-fourth embodiment of the present disclosure is applied to the puncture apparatus.

Referring to this figure, the twenty-fourth embodiment of the medical tube and a method of placing an implant indwelling according to the present disclosure will be described below. In the following, description will be made primarily of differences from the aforementioned embodiments, and descriptions of the same items as above will be omitted.

The twenty-fourth embodiment is the same as the twenty-third embodiment above, except for differences in the configuration of wrapping material and/or medical tube.

As depicted in FIG. 63, in the present embodiment, a joint section 921 of a wrapping material 92 is a part which has a hardness equal to or higher than that of a main body 31, and which is formed at an outer circumferential portion thereof with a plurality (in the configuration illustrated, two) of projecting pieces 926. The joint section 921 can be joined to a proximal surface of the main body 31, with the projecting pieces 926 at an end surface of the joint section 921 attaching the proximal surface. By this, the joined state is maintained, and the main body section 911 of the implant main body 91 can be transferred from inside the wrapping material 92 into the main body 31 in a clean state.

FIG. 64 depicts views a longitudinal sectional view and a cross-sectional view for explaining an operating procedure of a puncture apparatus in the case where a medical tube according to a twenty-fifth embodiment of the present disclosure is applied to the puncture apparatus.

Referring to this figure, the twenty-fifth embodiment of the medical tube and a method of placing an implant indwelling according to the present disclosure will be described below. In the following, description will be made primarily of differences from the aforementioned embodiments, and descriptions of the same items as above will be omitted.

The twenty-fifth embodiment is the same as the twenty-third embodiment above, except for differences in the configuration of wrapping material and/or medical tube.

As illustrated in FIG. 64, in the present embodiment, a joint section 921 of a wrapping material 92 is a cylindrical part having a hardness equal to or higher than that of a main body 31. The diameter of the joint section 921 is substantially equal to the major diameter of a proximal surface of the main body 31, which is flat shaped in cross section. Such a joint section 921 can be joined to the proximal surface of the main body 31, with an end surface of the joint section 921 attaching proximal surface. By this, the joined state is maintained, and, accordingly, the main body section 911 of the implant main body 91 can be transferred from inside the wrapping material 92 into the main body 31 in a clean state.

FIG. 65 depicts a longitudinal sectional view and a cross-sectional view for explaining an operating procedure of a puncture apparatus in the case where a medical tube according to a twenty-sixth embodiment of the present disclosure is applied to the puncture apparatus.

Referring to this figure, the twenty-sixth embodiment of the medical tube and a method of placing an implant indwelling according to the present disclosure will be described below. In the following, description will be made primarily of differences from the aforementioned embodiments, and descriptions of the same items as above will be omitted.

The twenty-sixth embodiment is the same as the twenty-third embodiment above, except for differences in the configuration of wrapping material and/or medical tube.

As illustrated in FIG. 65, in the present embodiment, a proximal portion as a joint portion of a main body 31 is a part which is formed at an inner circumferential portion thereof with a plurality (in the configuration illustrated, two) of projecting pieces 315. The projecting pieces 315 attach an end surface of a joint section 921 of a wrapping material 92, whereby a joined state is established. As a result, the main body section 911 of the implant main body 91 can be transferred from inside the wrapping material 92 into the main body 31 in a clean state.

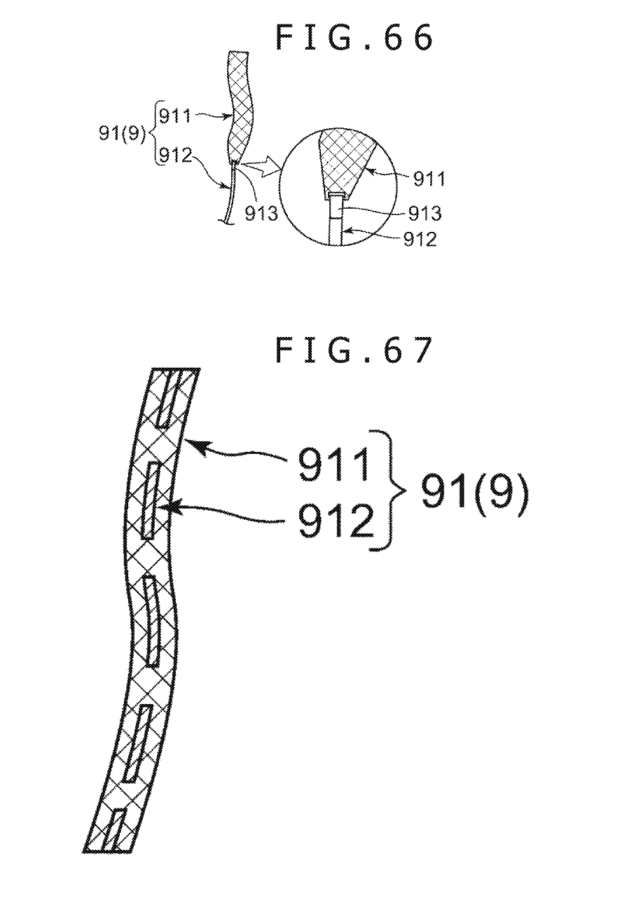

FIG. 66 is a perspective view of an implant to be used with a medical tube according to a twenty-seventh embodiment of the present disclosure.

Referring to this figure, the twenty-seventh embodiment of the medical tube and a method of placing an implant indwelling according to the present disclosure will be described below. In the following, description will be made primarily of differences from the aforementioned embodiments, and descriptions of the same items as above will be omitted.

The twenty-seventh embodiment is the same as the first embodiment above, except for differences in the configuration of implant.

As depicted in FIG. 66, in the present embodiment, an implant main body 91 has a configuration wherein an end portion of a ribbon 912 is folded back to be a folded-back portion 913. The folded-back portion 913 is made to penetrate a main body section 911, whereby the implant main body 91 and the ribbon 912 are interlocked to each other.

Releasing the interlock can be performed by extending the folded-back portion 913 in a direction opposite to the folding-back direction.

Note that at least the folded-back portion 913 of the ribbon 912 is preferably configured by use of a plastically deformable metallic sheet.

FIG. 67 is a perspective view of an implant to be used with a medical tube according to a twenty-eighth embodiment of the present disclosure.

Referring to this figure, the twenty-eighth embodiment of the medical tube and a method of placing an implant indwelling according to the present disclosure will be described below. In the following, description will be made primarily of differences from the aforementioned embodiments, and descriptions of the same items as above will be omitted.

The twenty-eighth embodiment is the same as the first embodiment, except for differences in the configuration of implant.

As depicted in FIG. 67, in the present embodiment, an implant main body 91 is in an assembled state wherein a ribbon 912 is inserted and passed through a main body section 911 along the longitudinal direction of the main body section 911. In this assembled state, the implant main body 91 can be moved from inside the wrapping material 92 into the sheath 30. After the movement, in addition, the ribbon 912 can be drawn out of the main body section 911.

Note that the ribbon 912 is preferably formed from a material selected from among various metallic materials and various resin materials.

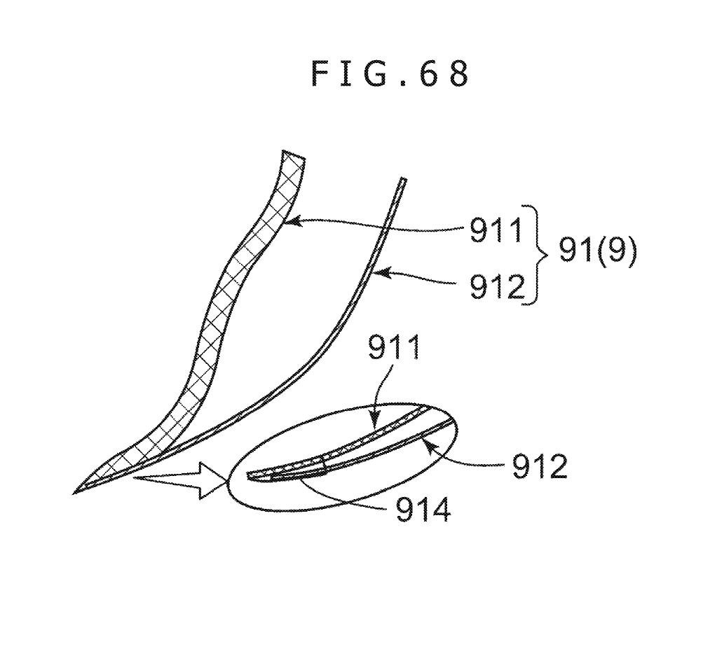

FIG. 68 is a perspective view of an implant to be used with a medical tube according to a twenty-ninth embodiment of the present disclosure.

Referring to this figure, the twenty-ninth embodiment of the medical tube and method of placing an implant indwelling according to the present disclosure will be described below. In the following, description will be made primarily of differences from the aforementioned embodiments, and descriptions of the same items as above will be omitted.

The twenty-ninth embodiment is the same as the first embodiment above, except for differences in the configuration of implant.

As illustrated in FIG. 68, in the present embodiment, an implant main body 91 is provided with a bag-shaped bag portion 914 at a distal portion of a main body section 911, and is in an assembled state wherein a distal portion of a ribbon 912 is inserted in the bag portion 914. When the ribbon 912 is pushed into the sheath 30 in the assembled state, the main body section 911 can be moved together with the ribbon 912 from inside the wrapping material 92 into the sheath 30. After the movement, in addition, the ribbon 912 can be drawn out of the bag portion 914.

Note that the ribbon 912 is preferably formed from a material selected from among various metallic materials and various resin materials.

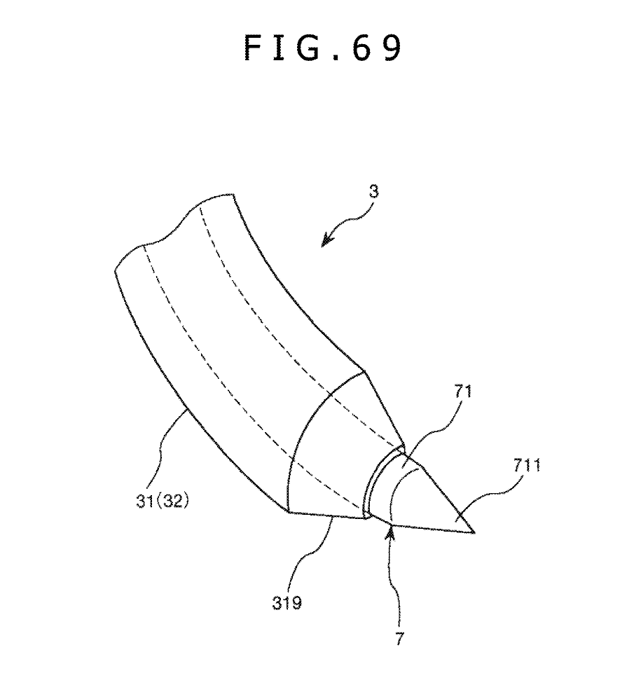

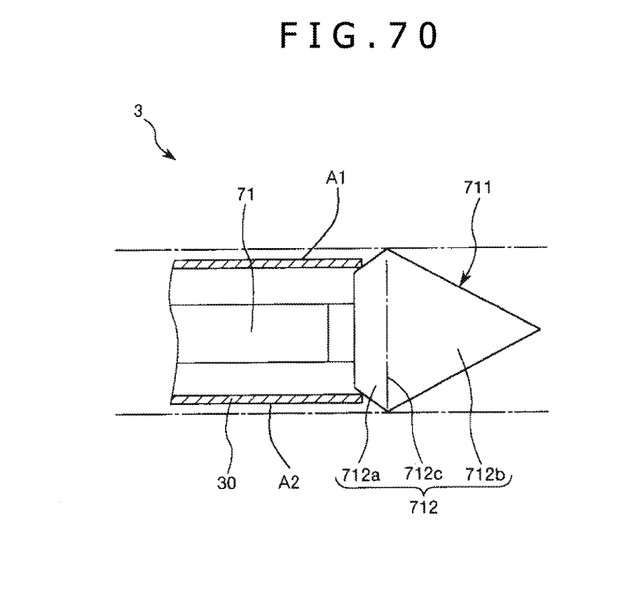

FIG. 69 is a perspective view illustrating a medical tube according to a thirtieth embodiment of the present disclosure. FIG. 70 is a sectional view depicting a modification of the medical tube depicted in FIG. 69.

Referring to these figures, the thirtieth embodiment of the medical tube, a medical device set, and a method of placing an implant indwelling according to the present disclosure will be described below. In the following, description will be made primarily of differences from the aforementioned embodiments, and descriptions of the same items as above will be omitted.

The thirtieth embodiment is the same as the aforementioned first embodiment, except mainly for differences in the configuration of puncture member.

As depicted in FIG. 69, in the present embodiment, a puncture member 3 is composed of a sheath 30. In other words, the puncture member 3 is configured by omitting the needle body 35 from the puncture member 3 in the aforementioned first embodiment. In addition, in a state (initial state) where an insertion section 71 is inserted in the puncture member 3, a distal portion 711 as a distal portion of the insertion section 71 is protruding from a distal-side opening of a main body 31. The distal portion 711 protruding from the main body 31 can also function as a needle tip of the puncture member 3. With the distal portion 711 of the insertion section 71 thus functioning also as the needle body of the puncture member 3, a reduction in the number of members can be contrived, as compared with the aforementioned first embodiment, for example. In addition, when the puncture member 3 is made to puncture a living body and the insertion section 71 is drawn out of the puncture member 3, the distal-side opening of the main body 31 can be opened. In other words, unlike in the aforementioned first embodiment, in the present embodiment it is unnecessary to detach the needle body 35 in order to open the distal-side opening of the main body 31, and, accordingly, the operation can be carried out relatively smoothly. In addition, the outside diameter of the insertion section 71 and the inside diameter of the distal-side opening of the main body 31 are set to be substantially the same, so that slippage of the insertion section 71 relative to the main body 31 is prevented and, hence, operability can be enhanced.

In addition, the main body 31 is provided at its distal portion with a tapered section 319 where its outside diameter gradually increases along the proximal direction from its distal-side opening. The tapered section 319 can function as a dissecting section which, as the distal portion 711 of the insertion section 71 punctures a living body, dissects the living body in the manner of gradually expanding the living body, following the distal portion 711.

Note that while the taper angle of the tapered section 319 and the taper angle of the distal portion 711 may be the same, they are preferably different from each other as depicted in FIG. 69. In this case, it can be preferable that the taper angle of the tapered section 319 is smaller than the taper angle of the distal portion 711, which configuration helps enable a smooth puncture.

According to the thirtieth embodiment as above, also, the same or equivalent effects to those of the aforementioned first embodiment can be produced.

In addition, as a modification of the present embodiment, the following configuration may be mentioned. As depicted in FIG. 70, a puncture member 3 is composed of a sheath 30. In other words, the puncture member 3 is configured by omitting the needle body 35 from the puncture member 3 in the aforementioned first embodiment. In addition, in a state (initial state) where an insertion section 71 is inserted in the puncture member 3, a distal portion 711 as a distal portion of the insertion section 71 is protruding from a distal-side opening of a main body 31.

The distal portion 711 is provided in a detachable manner in relation to the insertion section 71, through screw engagement, fitting or the like. In addition, the distal portion 711 has a needle tip 712 protruding from the distal end of the sheath 30. The needle tip 712 has a flat shape modeled after the sheath 30. In addition, the needle tip 712 can include a gradually increasing area section 712a where its cross-sectional area gradually increases toward its distal end, and a gradually decreasing area section 712b where its cross-sectional area gradually decreases toward its distal end. The minor axis of a boundary 712c between the gradually increasing area section 712a and the gradually decreasing area section 712b is longer than the minor axis at the distal end of the sheath 30, and the major axis of the boundary 712c is longer than the major axis at the distal end of the sheath 30, which can help ensure that the inside of a living body can be punctured substantially by only the needle tip 712. Therefore, puncture resistance can be reduced, and a living body can be punctured relatively smoothly. Note that the minor axis of the boundary 712c may be equal to the minor axis at the distal end of the sheath 30, and the major axis of the boundary 712c may be equal to the major axis at the distal end of the sheath 30.

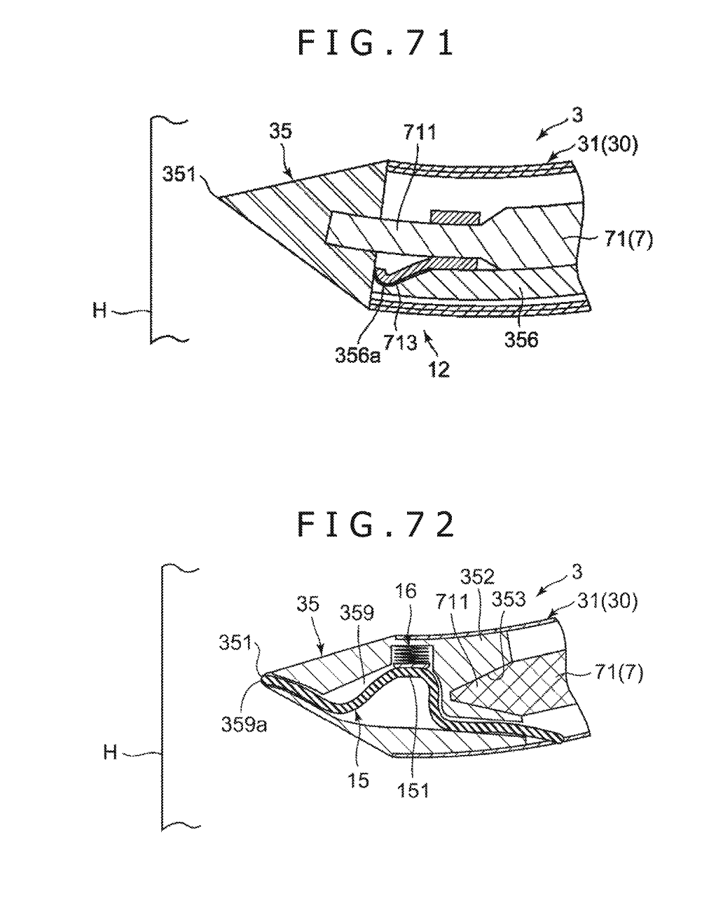

FIG. 71 is a sectional view of a puncture apparatus in the case where a medical tube according to a thirty-first embodiment of the present disclosure is applied to the puncture apparatus.

Referring to this figure, the thirty-first embodiment of the medical tube and a method of placing an implant indwelling according to the present disclosure will be described below. In the following, description will be made primarily of differences from the aforementioned embodiments, and descriptions of the same items as above will be omitted.

The thirty-first embodiment is the same as the first embodiment above, except for differences in the configuration of puncture member.

As depicted in FIG. 71, in the present embodiment, an engaging piece 713 of an operating member 7 constituting a locking section 12 is provided in the number of only one, and only one elastic piece 356 is provided accordingly. The elastic piece 356 is preferably circular arc shaped in cross section. In addition, the elastic piece 713 is disposed on the outer side of the curve of a sheath 30. A puncture resistance at the time when a puncture member 3 punctures a living body is exerted mainly on the outer side of the curve of the sheath 30. In view of this, the engaging piece 713 disposed on the outer side of the curve is preferably made to be thick (large in diametric size). In addition, in this case, an elastic force of the engaging piece 713 is also increased, so that an engaging force of the needle body 35 with respect to the first engaging portion 356a is also increased.

In addition, since only one elastic piece 356 is provided, the elastic piece 356 itself can be made to be thicker (larger in diametric size) than in the case where two elastic pieces 356 are provided. As a result, the strength of the elastic piece 356 increases. In addition, since the number of the elastic piece(s) 356 arranged is reduced, the sheath 30 can be made smaller in diameter accordingly.

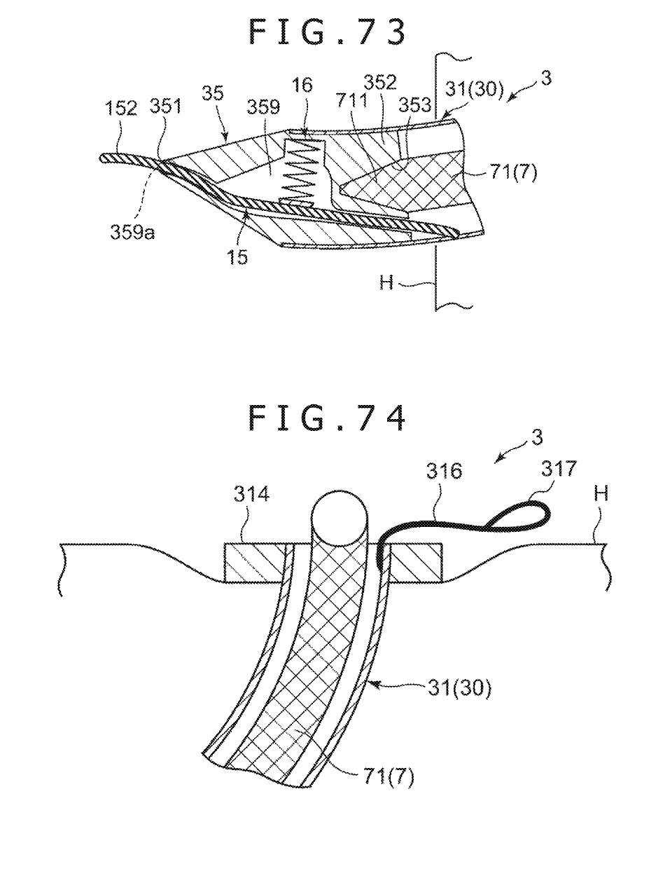

FIGS. 72 and 73 are views for explaining an operating procedure of a puncture apparatus in the case where a medical tube according to a thirty-second embodiment of the present disclosure is applied to the puncture apparatus. FIG. 74 is a view illustrating a state on a proximal side of a puncture member in FIG. 73.

Referring to these figures, the thirty-second embodiment of the medical tube and a method of placing an implant indwelling according to the present disclosure will be described below. In the following, description will be made primarily of differences from the aforementioned embodiments, and descriptions of the same items as above will be omitted.

The thirty-second embodiment is the same as the first embodiment above, except for differences in the configuration of puncture member.

As illustrated in FIGS. 72 and 73, in the present embodiment, a sheath 30 is provided in a distal portion thereof with an elastic linear (filamentous) body 15 which is supported and fixed, or joined, to the distal portion from inside. As depicted in FIG. 72, the linear body 15 can include a curved portion 151 which is curved in a natural state with no external force exerted thereon. In addition, the curved portion 151 is in the state of being accommodated in an internal space 359 of a needle body 35.

The needle body 35 can include an opening portion 359a where the internal space 359 opens at a needle tip 351. In addition, a compression coil spring 16 for biasing the curved portion 151 in a stretching direction is accommodated in the internal space 359 in a compressed state.

As depicted in FIG. 72, during when a puncture member 3 punctures a living body tissue, a distal end of the linear body 15 is pressed toward the proximal side due to a puncture resistance from the living body tissue, so that protrusion of the distal end from the opening portion 359a of the needle body 35 is restricted. As a result, the curved portion 151 can maintain the curved shape in the internal space 359, against the biasing force of the compression coil spring 16.

When the needle body 35 protrudes from the body surface H, as depicted in FIG. 73, the linear body 15 is released from the puncture resistance from the living body tissue. As a result, the compression coil spring 16 can bias the curved portion 151 completely, so that the curved portion 151 is stretched. The linear body 15 can protrude from the opening portion 359a of the needle body 35, by an amount according to the stretching. This protruding portion 152 functions as a grasping portion to be grasped and pulled at the time of dividing the sheath 30.

Note that the linear body 15 is curved in a natural state in the present embodiment, this is not restrictive. For example, the linear body 15 may be substantially straight in shape in the natural state.

As depicted in FIG. 74, on the proximal side of the puncture member 3, a flange portion 314 provided at a proximal portion of the sheath 30 attaches the body surface H. In addition, a cord 316 is supported and fixed to the proximal portion of the sheath 30 from inside. A looped finger hook portion 317 is provided at an end portion of the cord 316. At the time of dividing (separating) the sheath 30, a dividing operation can be conducted by putting a finger in the finger hook portion 317.

While the medical tube, the medical device set, and the method of placing an implant indwelling according to the present disclosure have been described above with reference to the preferred embodiments illustrated in the attached drawings, the present disclosure is not limited to the embodiments. The configuration of each component of the medical tube can be replaced by any configuration that has a function equivalent to the original. In addition, any other structure may be added to the configuration according to the present disclosure. An arbitrary step or steps may be added to the method of placing an implant indwelling according to the present disclosure.

In addition, in regard of the medical tube, the medical device set, and the method of placing an implant indwelling according to the present disclosure, arbitrary two or more of the configurations or features of the aforementioned embodiments may be combined in a desired manner.

In addition, while the needle body is retained on the main body in a freely detachable manner in the above embodiments, this configuration is not restrictive. For example, the needle body may be fixed to the main body, like in a configuration wherein the main body and the needle body are formed integrally. In this case, the distal-side opening of the main body can be opened by cutting the needle body by use of a pair of scissors or the like, after a living body is punctured by the puncture member and the needle body is protruded to the outside of the living body.

In addition, while a configuration wherein the main body of the puncture member is disposed inside a living body and thereafter the implant main body is inserted into the main body has been described in the above embodiments, this configuration is not restrictive. A configuration may be adopted in which the implant main body is accommodated in the puncture member (main body) from the beginning. In this case, it is preferable that, for example, a string located on the needle tip side, of two strings possessed by the implant main body, is preliminarily fixed to the needle tip, which helps ensure that when the needle tip is detached from the main body, the string can be protruded to the outside of the main body in an attendant manner. As a result, the subsequent fine adjustment of the disposition of the implant main body and the like can be performed smoothly.

In addition, while a case where the puncture apparatus is applied to an apparatus for use in embedding in a living body an embeddable implant for treatment of female urinary incontinence has been described in the above embodiments, the use of the puncture apparatus is not limited to the described one.

For example, the target of the application of the present disclosure can include excretory disorders attendant on the weakening of the pelvic floor muscle group (urinary urgency, frequent urination, urinary incontinence, fecal incontinence, urinary retention, strangury or the like), and pelvic floor disorders including pelvic organ prolapse, vesicovaginal fistula, urethrovaginal fistula, pelvic pain or the like. In the pelvic organ prolapse, there are included disorders of cystocele, enterocele, rectocele, hysterocele and the like. Alternatively, there are included such disorders as anterior vaginal prolapse, posterior vaginal prolapse, vaginal apical prolapse, vaginal vault prolapse and the like in which the naming method thereof is based on the prolapsed vaginal-wall part.

Also, overactive tissues include bladder, vagina, uterus, bowel and the like. Less active tissues include bones, muscles, fascias, ligaments and the like. In particular, in the case of pelvic floor disorders, the less active tissues include an obturator fascia, a coccygeus fascia, a cardinal ligament, an uterosacral ligament, a sacrospinous ligament and the like.

For the procedure for interlocking an overactive tissue in the pelvic floor disorder with the less active tissue, there are included a retropubic sling surgery, a transobturator sling surgery (transobturator tape (TOT) surgery), a tension-free vaginal mesh (TVM) surgery, a uterosacral ligament suspension (USLS) surgery, a sacrospinous ligament fixation (SSLF) surgery, an iliococcygeus fascia fixation surgery, a coccygeus fascia fixation surgery, and the like.

As has been described above, the medical tube disclosed herein is a medical tube in which an implant to be placed indwelling in a living body can be inserted and which can include an extension section that extends the overall length of the medical tube. Therefore, an operation of inserting and passing an implant into and through the medical tube can be performed easily and reliably.

Therefore, the medical tube according to the present disclosure has industrial applicability.

The detailed description above describes a medical tube, a medical device set, and a method of placing an implant indwelling. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A medical tube configured so that an implant to be placed indwelling in a living body can be inserted in the medical tube, the medical tube comprising:
an extension section which extends an overall length of the medical tube.

2. The medical tube according to claim 1, comprising:
a tube main body including a plurality of tube shaped members interlocked along a longitudinal direction of the medical tube, the tube shaped members communicating with one another, and
wherein the extension section supports adjacent tube shaped members so that the adjacent tube shaped members are movable relative to one another along the longitudinal direction.

3. The medical tube according to claim 1, comprising:
a tube main body including a deformation portion provided at an intermediate part in a longitudinal direction of the medical tube, the deformation portion deformed so as to extend along the longitudinal direction, a deformed state of the deformation portion being maintained, and
wherein the deformation portion functions as the extension section.

4. The medical tube according to claim 1,
wherein at least part of the medical tube is rigid, and lumens of the medical tube are communicating after an extending operation by the extension section.

5. The medical tube according to claim 2,
wherein the tube main body is configured to be separable, and has a detection section capable of detecting a separation position of the tube main body in a connected state of the tube main body.

6. The medical tube according to claim 3,
wherein the tube main body is configured to be separable, and has a detection section capable of detecting a separation position of the tube main body in a connected state of the tube main body.

7. A medical device set comprising:
a medical tube configured so that an implant to be placed indwelling in a living body can be inserted in the medical tube, the medical tube including an extension section which extends an overall length of the medical tube; and
an operation mechanism for operation to actuate an extension mechanism.

8. The medical device set according to claim 7,
wherein the operation mechanism includes a pressing member which is inserted into the medical tube from a first end side thereof, the pressing member pressing the medical tube toward a second end side thereof.

9. The medical device set according to claim 8,
wherein the pressing member is drawn out after an operation of the extension mechanism, and
a movement preventing mechanism is provided which prevents the medical tube from moving together with the pressing member when the pressing member is drawn out.

10. The medical device set according to claim 7,
wherein the medical tube comprises a tube main body including a plurality of tube shaped members interlocked along a longitudinal direction of the medical tube, the tube shaped members communicating with one another, and
wherein the extension section supports adjacent tube shaped members so that the adjacent tube shaped members are movable relative to one another along the longitudinal direction.

11. The medical device set according to claim 7,
wherein the medical tube comprises a tube main body including a deformation portion provided at an intermediate part in a longitudinal direction of the medical tube, the deformation portion deformed so as to extend along the longitudinal direction, a deformed state of the deformation portion being maintained, and
wherein the deformation portion functions as the extension section.

12. The medical device set according to claim 7,
wherein at least part of the medical tube is rigid, and lumens of the medical tube are communicating after an extending operation by the extension section.

13. The medical device set according to claim 10,
wherein the tube main body is configured to be separable, and has a detection section capable of detecting a separation position of the tube main body in a connected state of the tube main body.

14. A method of placing an implant indwelling in a living body by temporarily inserting the implant in a medical tube, the method comprising:
preliminarily extending an overall length of the medical tube prior to placing the implant indwelling.

15. The method according to claim 14, comprising:
extending the overall length of the medical tube with an extension section.

16. The method according to claim 15, comprising:
a tube main body including a plurality of tube shaped members interlocked along a longitudinal direction of the medical tube, the tube shaped members communicating with one another, and
supporting adjacent tube shaped members with the extension section so that the adjacent tube shaped members are movable relative to one another along the longitudinal direction.

17. The method according to claim 15, comprising:
a tube main body including a deformation portion provided at an intermediate part in a longitudinal direction of the medical tube, the deformation portion deformed so as to extend along the longitudinal direction, a deformed state of the deformation portion being maintained, and
wherein the deformation portion functions as the extension section.

18. The method according to claim 15,
wherein at least part of the medical tube is rigid, and lumens of the medical tube are communicating after an extending operation by the extension section, and
wherein the tube main body is configured to be separable, and has a detection section capable of detecting a separation position of the tube main body in a connected state of the tube main body.
